(19) United States
(12) Patent Application Publication
(10) Pub. No.: US 2024/0103009 A1
(43) Pub. Date: Mar. 28, 2024

(54) NON-TERMINAL ANTIBODY DISCOVERY METHODS AND SINGLE CELL ASSAYS

(71) Applicant: AMGEN INC., Thousand Oaks, CA (US)

(72) Inventors: Karyn E. MCFADDEN, Vancouver (CA); Brian M. CHAN, Port Coquitlam (CA); Christopher MURAWSKY, Roberts Creek (CA); Aaron George WINTERS, Ventura, CA (US); Daniel SANTOS, Surrey (CA)

(73) Assignee: AMGEN INC., Thousand Oaks, CA (US)

(21) Appl. No.: 18/275,855

(22) PCT Filed: Feb. 4, 2022
(86) PCT No.: PCT/US2022/015279

§ 371 Date: Aug. 4, 2023

Related U.S. Application Data
(60) Provisional application No. 63/146,135, filed on Feb. 5, 2021.

(51) Int. Cl. G01N 33/68; C07K 16/00; C12N 5/0781

(52) U.S. Cl. CPC G01N 33/6854; C07K 16/00; C12N 5/0635; C07K 2317/14; C07K 2317/92

(57) ABSTRACT

Provided herein are methods of monitoring for the production of select antibodies in a non-human animal, comprising (a) immunizing a non-human animal with an immunogen; (b) obtaining a blood sample comprising antibody secreting cells (ASCs) from said non-human animal; and (c) individually assaying ASCs present in the blood sample, or a fraction thereof, for the production of select antibodies. Methods of guiding antibody production in a non-human animal for the production of select antibodies are also provided. In exemplary embodiments, the method comprises performing a cycle of (a) to (c), as above, and repeating the cycle when the percentage of ASCs producing select antibodies is below a threshold. In various aspects, the cycle is repeated until the percentage of ASCs producing select antibodies is at or above a threshold. Single cell assays are further provided herein.

### CROSS REFERENCE TO RELATED APPLICATION

This application claims priority to U.S. Provisional Application No. 63/146,135, filed Feb. 5, 2021, the entire contents of which is incorporated herein by reference.

### BACKGROUND

Traditional animal-based antibody discovery methods involve the sacrifice of animals based on polyclonal serum titers against protein targets with varying degrees of complexity. While some antibody discovery campaigns have simple design goals (e.g., bind to the target), most are more complex and require the desired antibodies to have a variety of features (e.g., cross reactivity, bind to a particular epitope, bind with a specific affinity, etc.). Traditional antibody discovery approaches rely on the interrogation of the polyclonal secreted antibody (serum) response to select animals for B-cell harvest and antibody generation. The “serum titer” approach is less than ideal since it measures the total reactivity of all the secreted antibodies (i.e., it is a polyclonal mixture) and cannot be used to identify the B-cell source of the detected antibodies (i.e., there is both a physical and temporal disconnect from the source B-cell). The lack of a direct connection between phenotype (the antibody titer measurement) and genotype (the responsible source B-cell encoding the antibody) makes interpreting the quality of the B-cell response difficult. Aside from determining whether there is soluble, antigen-specific antibody in the serum, it is difficult to obtain additional useful information from this polyclonal analysis that can aid animal selection.

Additionally, the traditional methodology is terminal with regard to the animal, and thus represents a ‘one-time’ attempt to capture the animal's relevant B-cell repertoire. Failure to capture this repertoire, which may be caused by technical problems, selection of animals with suboptimal antibody production, and/or the lack of sampling depth of the repertoire (i.e., poor efficiency of traditional viral immortalization and hybridoma processes resulting in the fusion of a very small fraction of the B cell repertoire (less than 0.1%)), results in the waste of valuable resources and forces the use of an alternative immune animal or an entirely new immunization campaign. Furthermore, the traditional methods preclude the possibility of a continual process of leveraging the immune system of the same animal to evolve the antibody response.

Despite these limitations, the traditional methods are widely used, in part, because they allow the capture of an acceptable fraction of the immune repertoire and provide a renewable source of antibody that can easily be scaled to accommodate downstream assays.

There is an increasing number of situations where these traditional methods are too slow to meet project timelines, capture the wrong B-cell population, insufficiently sample the B-cell repertoire, or do not allow real-time monitoring of the evolving B-cell response. In addition, the challenging nature of many antibody target classes (e.g. complex membrane proteins, targets with minimal epitope space, proteins that are highly similar to orthologs, etc.) can make it difficult to raise B-cell responses in animals due to a lack of robust immunogenicity. Coupled with the extreme complexity of some antibody design goals, generating immune animals with the desired immune profiles (i.e., B-cell repertoires) can be difficult.

In view of the foregoing, more efficient antibody discovery methods are needed. For example, antibody discovery methods that can better position traditional animal immunization and B-cell methods for successful antibody discovery would greatly enhance animal-based antibody discovery.

### SUMMARY

Provided for the first time are the rationale, experimental methods, and data demonstrating techniques useful in antibody discovery. In exemplary aspects, the methods involve the identification of antigen-specific antibodies directly from the peripheral blood of a living, non-human animal. Advantageously, such methods provided herein allow for antibody discovery without the need for animal sacrifice, unlike traditional methods which rely on animal euthanasia followed by immune organ harvest (e.g., spleen, lymph nodes, and bone marrow). Because such methods are non-terminal (e.g., do not involve the euthanasia of the antibody-producing animals), the methods may be repeated multiple times in the same animal(s) until, for instance, an antibody of interest is obtained. The ability to repeat the method in the same animal(s) has several advantages over traditional methods. For example, repeating the method in the same animals(s) reduces the overall cost of the antibody discovery process. Also, since the animals are kept alive, the presently disclosed methods allow for real-time, in-life sampling of the antibody repertoire, such that if, for example, the animals do not produce a B cell expressing the target antibody of interest, strategic adjustments to the immunization protocol (used in the next immunization) may be made based on the observed B-cell response (from the prior immunization). The methods of the present invention thereby permit rational repertoire shaping and/or purposeful steering of the immune response to match antibody design goals. Exemplary processes of the present disclosure are illustrated in FIGS. 1B-IE. FIG. 1B illustrates an exemplary non-terminal method for monitoring immune responses comprising screening of single cells obtained from a blood sample of immunized animals. Based on the outcome of the single cell screening, the animal may be subjected to a repeat round of immunization (e.g., an alternative immunization) followed by single cell screening of cells from the blood sample obtained from the immunized animal, or may undergo tissue harvest if the screening determines that the animal exhibits the desired phenotype. FIG. 1C illustrates an exemplary non-terminal method of monitoring for the production of select antibodies, wherein antibody secreting cells (ASCs) purified from a blood sample obtained from an immunized animal are screened at the single cell level. The process is repeated until design goals are met and/or select antibodies are produced. FIG. 1D illustrates an exemplary non-terminal method of guiding antibody production for select antibody production wherein a primary strategy is used to immunize animals, and the ASCs obtained from PBMCs isolated from the immunized animal are screened for the desired phenotype. If the screening determines that the design goal is not met, then the animal is immunized with an alternative strategy (e.g., that differs from the primary strategy) and, the ASCs obtained from PBMCs isolated from the immunized animal are screened for the desired phenotype. The process is repeated until the screening determines that the design goal is met. When and if the design goal is met, terminal tissue may be harvested for antibody rescue using hybridoma, single cell platforms or sequence-based discovery. FIG. 1E illustrates an exemplary non-terminal method of screening animals and B-cell profiling wherein a series of animals is immunized with an immunogen and ASCs obtained from a blood sample obtained from each animal are screened and a B-cell repertoire is profiled. In various aspects of the exemplary processes, antibody secreting cells (ASCs), e.g., plasmablasts, are purified from the peripheral blood of an immunized mouse and then screened at single cell resolution for the relevant activity or phenotype. Compared to the traditional hybridoma production process (illustrated in FIG. 1A), which typically requires about 8 weeks and requires a high level of technical skill, the process of the present disclosure is less labor-intensive and requires less time.

Accordingly, the present disclosure provides methods of monitoring for the production of select antibodies in a non-human animal. In exemplary embodiments, the method comprises (a) immunizing a non-human animal with an immunogen; (b) obtaining a blood sample comprising antibody secreting cells (ASCs) from said non-human animal; and (c) assaying, e.g., individually assaying, ASCs present in the blood sample, or a fraction thereof, for the production of select antibodies. In various instances, the method further comprises repeating (b) and (c) one or more times until design goals are met, e.g., until select antibodies are produced. FIG. 1C illustrates this exemplary aspect of the present disclosure. The present disclosure also provides methods of guiding antibody production in a non-human animal for the production of select antibodies. In exemplary embodiments, the method comprises (a) performing an initial immunization on a non-human animal with an immunogen; (b) obtaining a blood sample comprising ASCs from said non-human animal; (c) assaying, e.g., individually assaying, ASCs present in the blood sample, or a fraction thereof, for the production of select antibodies; and (d) performing a cycle of steps when the percentage of ASCs producing select antibodies is below a threshold, wherein the cycle comprises (i) performing a subsequent immunization on the non-human animal with an immunogen when the percentage of ASCs producing select antibodies is below a threshold, (ii) obtaining a blood sample comprising ASCs from said non-human animal, and (iii) assaying, e.g., individually assaying, ASCs present in the blood sample, or a fraction thereof, for the production of select antibodies.

In various aspects, the assaying comprises a single-cell, live-cell assay. As used herein, the phrase “individually assaying ASCs” means that the ASCs are assayed or examined at the single cell level or at a single cell resolution. In exemplary instances, “individually assaying ASCs” provide results relevant to a single ASC. Optionally, multiple ASCs are simultaneously assayed. In various aspects, multiple ASCs are simultaneously individually assayed. In exemplary aspects, the blood sample is obtained from the non-human animal in a non-terminal manner, e.g., the non-human animal is not killed during the blood sample collection. In exemplary instances, the method comprises performing a non-terminal blood draw from the non-human animal. In various instances, the method comprises applying the blood sample, or a fraction thereof, to a matrix and assigning a unique address of the matrix to each ASC. Optionally, a result of the assaying is the identification of each ASC producing select antibodies. In certain aspects, the result of the assaying is the identification of the unique address of each ASC producing select antibodies. In exemplary instances, the method comprises at least one cycle of (i) performing a subsequent immunization on the non-human animal with an immunogen when the percentage of ASCs producing select antibodies is below a threshold, (ii) obtaining a blood sample comprising ASCs from said non-human animal, (iii) assaying, e.g., individually assaying ASCs present in the blood sample, or a fraction thereof, for the production of select antibodies. Optionally, the cycle is repeated until the percentage of ASCs producing select antibodies, as assayed in (iii), is at or above the threshold. In various instances, the cycle is repeated at least two times.

The immunogen of the subsequent immunization may differ from the immunogen of the initial immunization in exemplary aspects. For instance, in exemplary aspects, each subsequent immunization differs from a prior immunization in that (A) a different immunogen, adjuvant, and/or immunomodulatory agent is administered to the non-human animal, (B) a different dose of the immunogen is administered to the non-human animal, (C) the time between each administration of the immunogen, adjuvant, immunomodulatory agent is different, and/or (D) the route of administration for each administration of immunogen, adjuvant, immunomodulatory agent is different. Optionally, a different immunogen is used each time the non-human animal is immunized. FIG. 1D illustrates an exemplary method of guiding antibody production for select antibody production.

The present disclosure further provides methods of producing select antibodies in a non-human animal. In exemplary embodiments, the method comprises guiding antibody production in a non-human animal for the production of select antibodies in accordance with the presently disclosed methods of guiding antibody production and then isolating the select antibodies and/or an ASC producing the select antibodies. In exemplary embodiments, the method comprises (a) performing an initial immunization campaign on a non-human animal with an immunogen; (b) obtaining a blood sample comprising antibody secreting cells (ASCs) from said non-human animal; (c) assaying, e.g., individually assaying, ASCs present in the blood sample, or a fraction thereof, for the production of select antibodies; (d) performing a cycle of steps when the percentage of ASCs producing select antibodies is below a threshold, wherein the cycle comprises (i) performing a subsequent immunization on the non-human animal with an immunogen when the percentage of ASCs producing select antibodies is below a threshold, (ii) obtaining a blood sample comprising ASCs from said non-human animal, and (iii) assaying, e.g., individually assaying, ASCs present in the blood sample, or a fraction thereof, for the production of select antibodies; and (e) isolating the select antibodies and/or an ASC producing the select antibodies. In various aspects, the method comprises (f) determining the nucleotide sequence encoding the heavy chain variable region of the select antibodies produced by an ASC (e.g., the isolated ASC producing the select antibodies) and the nucleotide sequence encoding the light chain variable region of the select antibodies produced by the ASC, (g) introducing into a host cell a first vector comprising the nucleotide sequence encoding the heavy chain variable region of the select antibodies and a second vector comprising the nucleotide sequence encoding the light chain variable region of the select antibodies, and (h) isolating the antibodies produced by the host cell.

In exemplary aspects, the assaying of the presently disclosed methods comprises (a) combining the ASCs within the matrix with reagents that bind to the select antibodies and produce a detectable signal, e.g., a fluorescent signal, upon binding to the select antibodies. In various aspects, the assaying of the presently disclosed methods comprises (a) combining the ASCs within the matrix with at least one reagent which binds to the Fc domain of the select antibodies and at least one reagent to which select antibodies bind (e.g., a reagent which binds to the antigen-binding domain of the select antibodies), wherein at least one of these reagents is attached to a detectable label. In exemplary instances, the ASCs are combined with a detection reagent which binds to the Fc domain of the select antibodies and comprises a first detectable label and a target to which select antibodies bind (e.g., a reagent which binds to the antigen-binding domain of the select antibodies). FIGS. 2A-2C illustrate exemplary assaying in the context of the presently disclosed methods. In various instances, the target is labeled by a second detectable label which is different from the first detectable label. In some instances, a capture reagent which binds to the Fc domain of the select antibodies and comprises a solid support is further combined with the ASCs, detection reagent and labeled target. In various instances, the method further comprises (b) assaying for the first detectable label and the second detectable label; and (c) identifying the positions within the matrix at which the first detectable label and the second detectable label are detected, wherein each identified position locates an individual ASC producing select antibodies. FIGS. 2A and 2B illustrate such exemplary assaying with a labeled target and a capture reagent. FIG. 2A illustrates the matrix as a well. FIG. 2B illustrates the matrix as a multi-pen chip or multi-well plate and each ASCs is positioned into a single pen or well. In various instances, the target is expressed by cells and the cells expressing the target are combined with the ASCs and the detection reagent. In exemplary aspects, the method further comprises (b) assaying for the first detectable label; and (c) identifying the positions within the matrix at which the first detectable label is detected, wherein each identified position locates an individual ASC producing select antibodies. FIG. 2C illustrates such exemplary assaying with a cell expressing the target. In exemplary instances, the assaying of the presently disclosed methods comprises (a) combining the ASCs within the matrix with (i) a capture reagent which binds to the select antibodies and comprises a solid support, (ii) a detection reagent which binds to the select antibodies and comprises a first detectable label, and (iii) a labeled target to which the select antibodies bind, wherein the labeled target comprises a second detectable label distinct from the first detectable label; (b) assaying for the first detectable label and for the second detectable label; and (c) identifying the positions within the matrix at which both the first detectable label and the second detectable label are detected, wherein each identified position locates an individual ASC producing select antibodies. Optionally, the capture agent comprises an antibody that binds to an antibody Fc domain attached to a solid support. The detection agent, in exemplary instances, comprises an antibody that binds to an antibody Fc domain attached to a first detectable label. In various aspects, the antibody that binds to an antibody Fc domain of the capture agent is the same antibody of the detection agent. In exemplary instances, the combining takes place in a well and the capture agent forms a monolayer in the well. In various aspects, the method comprises identifying the positions within the well at which both the first detectable label and the second detectable label are detected, wherein each identified position locates an individual ASC producing select antibodies.

The present disclosure additionally provides single-cell assays for identifying ASCs producing select antibodies. The present disclosure provides methods of assaying for ASCs producing select antibodies. In exemplary embodiments, the assay or method comprises (a) combining in a well (i) a blood sample obtained from a non-human animal immunized with an immunogen, or a fraction thereof, wherein the blood sample comprises ASCs, (ii) a detection reagent which binds to the select antibodies and comprises a first detectable label, and (iii) a target to which the select antibodies bind, wherein (A) the target is a labeled target comprising a second detectable label distinct from the first detectable label and a capture reagent which binds to the select antibodies and comprises a solid support is further combined in the well to form a monolayer in the well or (B) the target is expressed on the surface of cells and the cells are combined in the well to form a monolayer in the well; (b) assaying for the first detectable label and optionally assaying for the second detectable label, when the target is a labeled target; and (c) identifying the positions within the well at which the first detectable label is detected or the first and second detectable labels are detected, wherein each identified position locates an individual ASC producing select antibodies. In various aspects, the assay or method comprises (a) combining in a well (i) a blood sample obtained from a non-human animal immunized with an immunogen, or a fraction thereof, (ii) a capture reagent comprising an antibody that binds to an Fc of an antibody attached to a solid support, (iii) a detection reagent comprising an antibody that binds to an Fc of an antibody attached to a first detectable label, and (iv) a labeled target comprising the immunogen, or a portion thereof, attached to a second detectable label distinct from the first detectable label, wherein the capture agent forms a monolayer in the well; (b) assaying for the first detectable label; (c) assaying for the second detectable label; and (d) identifying the positions within the well at which both the first detectable label and the second detectable label are detected, wherein each identified position locates an individual ASC producing select antibodies. In various aspects, the assay or method comprises (a) combining in a well (i) a blood sample obtained from a non-human animal immunized with an immunogen, or a fraction thereof, (ii) a detection reagent which binds to the select antibodies, and (iii) cells expressing on the cell surface a target to which the select antibodies bind, wherein the cells are combined in the well to form a monolayer in the well, (b) assaying for the first detectable label; and (c) identifying the positions within the well at which both the first detectable label and the second detectable label are detected, wherein each identified position locates an individual ASC producing select antibodies.

In various aspects of the presently disclosed methods, the non-human animal is subjected to neither removal of one or more secondary lymphoid organs nor euthanasia. Also, in various instances, ASCs from the blood sample are not used in making hybridomas. In exemplary aspects, the non-human animal is one of a series of non-human animals, and a result of the assaying is the identification of the non-human animals having a percentage of ASCs producing select antibodies below the threshold and/or requiring further immunization. In alternative aspects, the method comprises sacrificing the non-human animal and harvesting tissues from the non-human animal, when the percentage of ASCs producing select antibodies is at or above a threshold. In various instances, the steps of the method are carried out on a series of non-human animals and the method comprises profiling the B-cell repertoire of the blood sample for each non-human animal of the series and selecting a subset of the series having a target B-cell profile. FIG. 1E illustrates such steps.

Rational immune repertoire generation and selection is a critical component in animal-based antibody discovery technologies. Despite the advancement from traditional B-cell immortalization to direct B-cell platforms such as (but not limited to) NanOBLAST (an antibody discovery process on a nanofluidic Beacon® device) and microencapsulation, the diversity and quality of the input B-cells continues to be an essential determining factor in meeting antibody design goals. Traditional approaches to evaluate immune animals rely on the interrogation of the polyclonal secreted antibody (serum) to evaluate immune responses and select animals for B-cell harvest and antibody generation. The “serum titer” approach is less than ideal since it measures the total reactivity of all the secreted antibodies and not the quality of the individual B-cell source of the detected antibodies. The lack of a direct connection between the antibody titer measurement and the responsible B-cell source makes interpreting the quality of the B-cell response difficult. Aside from determining whether there is soluble, antigen-specific antibody in the serum, it is difficult to obtain additional useful information from this polyclonal analysis that can aid animal selection or immune steering strategies. Provided herein are ASC assays to interrogate the B-cell response of an immune animal using samples derived from non-terminal peripheral blood that would address these challenges. Accordingly, the present disclosure provides a method of screening non-human animals for antibody secreting cells (ASCs) producing select antibodies. The method in exemplary embodiments comprises (a) immunizing a series of non-human animals with an immunogen; (b) obtaining a blood sample comprising ASCs from each non-human animal of the series; and (c) individually assaying ASCs present in the blood sample, or a fraction thereof, for the production of select antibodies, wherein, for each non-human animal of the series, a percentage of ASCs producing select antibodies is determined. In various aspects, the screening method further comprises selecting the non-human animal(s) for sacrifice and/or tissue harvest, when the percentage of ASCs producing select antibodies is at or above a threshold. In various aspects, the screening method further comprises selecting the non-human animal(s) for subsequent immunization, when the percentage of ASCs producing select antibodies is below a threshold. Accordingly, in various embodiments, the screening method identifies animals for sacrifice vs. animals for subsequent immunization based on the percentage of ASCs producing select antibodies.

Consistent with the foregoing, methods of selecting immunized non-human animals for subsequent immunization are provided. In exemplary embodiments, the method comprises monitoring for the production of select antibodies in a non-human animal in accordance with any one of the presently disclosed methods, wherein the method is carried out on a series of non-human animals, wherein for each non-human animal of the series the number of ASCs producing the select antibodies is identified, and selecting the animal for subsequent immunization when the percentage of ASCs producing select antibodies for an animal is below a threshold. Also provided herein are methods of selecting immunized non-human animals for euthanasia and secondary lymphoid harvest. In exemplary embodiments, the method comprises monitoring for the production of select antibodies in a non-human animal in accordance with any one of the presently disclosed methods, wherein the method is carried out on a series of non-human animals, wherein for each non-human animal of the series the number of ASCs producing the select antibodies is identified, and selecting the animal for euthanasia and secondary lymphoid harvest, when the percentage of ASCs producing select antibodies for an animal is at or above a threshold.

### BRIEF DESCRIPTION OF THE DRAWINGS

FIG. 1A is an illustration of a traditional transgenic mouse hybridoma generation method. FIG. 1B is an illustration of a non-terminal method for monitoring immune response. FIG. 1C is an illustration of a non-terminal method of monitoring for the production of select antibodies. FIG. 1D is an illustration of a non-terminal method of guiding antibody production for select antibody production. FIG. 1E is an illustration of a non-terminal method of screening animals and B-cell profiling.

FIG. 2A is an illustration of an application of an exemplary single cell assay for identifying ASCs which produce select antibodies. FIG. 2B is an illustration of another exemplary single cell assay for identifying ASCs which produce select antibodies. FIG. 2C is an illustration of yet another exemplary single cell assay for identifying ASCs which produce select antibodies.

FIG. 3 is an illustration of an antibody binding to an anti-idiotope antibody. Paratopes, idiotypes and idiotopes are shown.

FIG. 4 is a graph of polyclonal titers for sera obtained from the indicated mice immunized with Antibody 1.

FIG. 5A is an illustration of components of an exemplary single cell screen and FIG. 5B is an illustration of how the components of the single cell assay interact in the presence of an antibody that binds antigen. FIG. 5C is an illustration of individual pens holding an ASC secreting antibodies interacting with polystyrene beads to create a fluorescent “bloom”. IgG secretion and antigen-specific antibodies are detected by the assay.

FIG. 6 is an illustration of the dual blooms above individual pens holding a single cell, export of the cells to a well and PCR analysis for antibody cloning, expression, purification and analysis.

FIG. 7A is an illustration of a sandwich ELISA format used to select the appropriate pairs of antibodies. FIG. 7B is graph of the ELISA signal plotted as a function of concentration of Antibody 1. FIG. 7C is a graph of the PD1 functional plotted as a function of antibody concentration.

FIG. 8A is an image of green fluorescent spots at which ASCs secreting antibodies are located in a single well. FIG. 8B is an image of red fluorescent spots at which antigen-specific antibodies secreted by ASCs are located in a single well. FIG. 8C is an image of colored spots at which ASCs secreting antibodies are located in a single well, spots at which antigen-specific antibodies secreted by ASCs are located in a single well, and spots at which ASCs secreting antigen-specific antibodies are located in a single well. FIG. 8D is an exemplary image of transfected cells labeled with multiple fluorescent spots at which antigen expressed by the 293T cell is bound to antibody produced by the B cell and labeled with the goat anti-human Fc antibody labeled with Alexa 488.

FIG. 9 is a series of images of single cells of the indicated hybridoma clone (or irrelevant clone) with RFU on the green channel (top) representing antibody secretion or the red channel (bottom) representing antigen (EGFR) binding.

FIG. 10 is a graph of the RFU green/RFU red ratio plotted as a function of KD of the hybridoma.

FIG. 11 is a schematic of an immunization protocol used across all mice. The timing of bleeds and shifting of antigens is indicated.

FIG. 12 is a graph of the serum titers of the first bleed of Group 1 and 2 mice. The graph plots the human antigen titers vs the cyno antigen titers.

FIG. 13 is a series of images of single cells with RFU on the red channel (left) representing human antigen binding, green channel (middle) representing cyno antigen binding, and composite channel (right) representing human antigen and cyno antigen binding. Data from serum obtained from Bleed 1.

FIG. 14 is a graph of the percent of antigen positive ASCs of Group 1 (closed circles) and Group 2 (open circles) mice reacting to human antigen only, cyno antigen only, or both human and cyno antigens. Data from cells from Bleed 1 obtained by the single cell Incucyte screen.

FIG. 15 is a graph of the percent of antigen positive ASCs of Group 1 (closed circles) and Group 2 (open circles) reacting to human antigen only, cyno antigen only, or both human and cyno antigens. Data from cells from Bleed 2 obtained by the single cell Incucyte screen.

FIG. 16 is a graph of the percent of antigen positive ASCs of Group 1A (Human Boost) and Group 1B (Cyno boost) reacting to human antigen only, cyno antigen only, or both human and cyno antigens. Data from cells from Bleed 2 shown in closed circles and data from cells from Bleed 3 shown in open squares. Data obtained by the single cell Incucyte screen.

FIG. 17 is a graph of the change in cross-reactive ASC frequency (relative to Bleed 1) of Group 1A (human boost) and Group 1B (cyno boost). Data obtained by the single cell Incucyte screen.

FIG. 18 is a graph of the serum titer reactive to cyno antigen plotted as a function of serum titer reactive to human antigen. Percent cross reactive ASCs are noted. Animals of interest for selection for harvest are circled in red.

FIG. 19 is a schematic of an immunization campaign with immune steering toward production of human cyno cross-reactive antibodies which bind to both human and cyno subdomain orthologs of a multi-domain protein (antigen).

FIG. 20 is a graph of serum titer reactive to cyno antigen plotted as a function of serum titer reactive to human antigen. Serum from Bleed 1.

FIG. 21 is series of images of single cells at t=0 hr (bottom) and t=23 hrs (top) with RFU on the green and red channels for human only binders, cyno only binders, and human/cyno cross-reactive binders. Data from serum obtained from Bleed 1 using the single cell Incucyte screen.

FIG. 22 is a graph of the percent of antigen positive ASCs reacting to human antigen only, cyno antigen only, or both human and cyno antigens. Data from cells from Bleed 1 shown. Data obtained by the single cell Incucyte screen.

FIG. 23 is a graph of the change in cross-reactive ASC frequency (relative to irrelevant clone) in serum from Bleed 1 and Bleed 3.

FIG. 24 is a graph of the percent of ASCs secreting antibodies reactive to cyno antigen only (closed circles) or both cyno and human antigen (open squares) at Bleed 1 and Bleed 3. Data obtained by the single cell Incucyte screen.

FIG. 25 is a graph of the percent of human-cyno cross-reactive binders. Animals of interest for selection for harvest are noted in squares. Data obtained by the single cell Incucyte screen.

### DETAILED DESCRIPTION

B-Cell Function and Non-Terminal Monitoring and Steering of Antibody Production

Antigen-specific B-cells that have recently encountered antigen in the germinal centers (GCs) of the secondary lymphoid organs (e.g., spleen and lymph nodes) are stimulated to divide and commit to differentiate down multiple pathways. See, e.g., Klein and Dalla-Favera, Nature Reviews Immunol 8: 22-33 (2008)). The main B-cell lineage responsible for secreting antibodies into the serum in response to antigen challenge are plasma cells. Plasma cell differentiation begins in the secondary lymphoid organs where cell-cell interactions within the GCs force B-cells, expressing antibodies on their surface that are specific to antigen, to differentiate into immature plasma cells known as plasmablasts. Plasmablasts are rapidly dividing B-cells that produce and secrete soluble antibody. However, plasmablasts are transient in nature and require significant trophic support to survive and continue to proliferate. The main survival niche for plasmablasts is in the secondary lymphoid organs, but these queues are provisional and depend on the presence of cognate antigen.

B-cells use two main strategies to maintain long-term humoral memory to antigens: the formation of IgG+ memory B-cells and the formation of long-lived, mature plasma cells. Memory B-cells express a cell-surface-bound version of their cognate antibody, known as the B-cell receptor (BCR), but do not secrete soluble antibody. These cells take up residence in a variety of locations throughout the body and are abundant within the secondary lymphoid organs. Upon re-encounter with antigen, memory B-cells can be induced to proliferate (i.e. to generate clones of themselves) and to differentiate into antibody-secreting plasma cells. The other route to long-term memory is via the formation of long-lived, mature plasma cells. Mature plasma cells require very specialized survival niches that provide trophic support and can be found within inflamed tissue, in specialized structures associated with the gut (gut-associated lymphoid tissue-GALT) and within the bone marrow. See, e.g., Fairfax et al., Semin Immunol 20(1): 49-58 (2008). The local environment created by niche stromal cells provides the necessary signals to maintain the longevity of the terminally differentiated plasma cells.

For B-cells to take up residence in long-term stromal niches, they must migrate to these destinations via the blood. Indeed, after exposure to antigen in the GCs, differentiation into plasmablasts and subsequent proliferation within the secondary lymphoid organs, a wave of migratory plasmablasts can be detected in circulation. In mice, this groundswell of plasmablasts in the blood occurs 3-7 days post antigen exposure and declines with time as they home to their appropriate niches and differentiate into long-lived plasma cells.

Provided herein are methods involving the capture of recently-antigen-stimulated plasmablasts and plasma cells (antibody secreting cells, (ASC)), as they migrate through the blood and the identification of those cells producing antibodies of interest, e.g., select antibodies. Because the method of the present disclosure utilizes blood samples and the cellular milieu of blood is substantially less complex than the that of secondary lymphoid organs, particularly from the perspective of the B-cell lineage, the methods of the present disclosure are advantageously less complex. The methods of the present disclosure address the difficulties accessing this ASC population, which historically has been difficult due to their relatively low overall abundance.

Accordingly, the present disclosure provides methods of monitoring for the production of select antibodies in a non-human animal. In exemplary embodiments, the method comprises (a) immunizing a non-human animal with an immunogen; (b) obtaining a blood sample comprising antibody secreting cells ASCs from said non-human animal; and (c) assaying (optionally, individually assaying) ASCs present in the blood sample, or a fraction thereof, for the production of select antibodies. The present disclosure also provides methods of guiding antibody production in a non-human animal for the production of select antibodies. In exemplary embodiments, the method comprises (a) performing an initial immunization campaign on a non-human animal with an immunogen; (b) obtaining a blood sample comprising antibody secreting cells (ASCs) from said non-human animal; (c) assaying (optionally, individually assaying) ASCs present in the blood sample, or a fraction thereof, for the production of select antibodies; and (d) performing a cycle of steps when the percentage of ASCs producing select antibodies is below a threshold, wherein the cycle comprises (i) performing a subsequent immunization on the non-human animal with an immunogen when the percentage of ASCs producing select antibodies is below a threshold, (ii) obtaining a blood sample comprising ASCs from said non-human animal, and (iii) individually assaying ASCs present in the blood sample, or a fraction thereof, for the production of select antibodies. In exemplary aspects, the threshold is about 1% to about 10%, e.g., about 1%, about 2%, about 3%, about 4%, about 5%, about 6%, about 7%, about 8%, about 9%, or about 10%. In exemplary aspects, the threshold is 10%, 15%, 20%, 25%, 30%, 35%, 40%, 45%, or 50%. In alternative aspects, the threshold is greater than 50%, e.g., 55%, 60%, 65%, 70%, 75%, 80%, 85%, 90%, 95%, or higher.

In exemplary aspects of such methods, the non-human animal is subjected to neither euthanasia nor removal of one or more secondary lymphoid organs, or the animal is euthanized and harvested for secondary lymphoid organs only after the animal has been deemed as possessing sufficient numbers of ASCs producing the antibodies of interest, e.g., select antibodies. In exemplary aspects, the methods are carried out with a series of non-human animals. In various instances, a result of the assaying is the identification of the non-human animal(s) of the series having a percentage of ASCs producing select antibodies which is below a threshold and/or requiring further immunization. Such non-human animal(s) can then be subject to a cycle of steps ((d), above) in order to, e.g., increase the production of select antibodies.

In alternative instances, a result of the assaying is the identification of the non-human animal(s) of the series having a percentage of ASCs producing select antibodies which is at or above a threshold. Such non-human animal(s) can then be sacrificed and the tissues harvested from such non-human animal(s). In alternative aspects, when the percentage of ASCs producing select antibodies relative to the total number of ASCs assayed is at or above a threshold, the method comprises sacrificing the non-human animal and harvesting tissues from the non-human animal.

Accordingly, the presently disclosed methods of monitoring and guiding or steering of select antibody production are highly efficient, as fewer animals (e.g., those with percentage of ASCs producing select antibodies below a threshold) are unnecessarily killed and a greater percentage of immunized animals ultimately yield select antibodies. Also, in various instances, such presently disclosed methods do not include generating hybridomas, and, therefore, are advantageously less time- and material-consuming.

Immunization

In various aspects of the present disclosure, the method comprises immunizing a non-human animal with an immunogen. As used herein, the term “immunizing” refers to performing or carrying out an “immunization campaign” or “immunization protocol” or “campaign” to mount an immune response against said immunogen. In exemplary aspects, the immune response comprises a B-cell immune response and/or a humoral immune response against said immunogen. In exemplary aspects, the immune response mounted in the non-human animal comprises the production of antibody-secreting cells (ASCs), e.g., antibody-secreting plasma cells, plasmablasts, plasma cells (e.g., rapidly dividing B cells that produce and secrete high level of soluble antibody). In various instances, the immune response comprises migratory ASCs (e.g., plasma cells, plasmablasts) which migrate through the blood to secondary lymphoid organs. In various aspects, the secondary lymphoid organ is a lymph node (e.g., popliteal, inguinal, mesenteric, and brachial), spleen, a Peyer's patch, or a mucosal tissue. In exemplary instances, the ASCs are produced about 1-7 days after antigen exposure. Optionally, ASCs, e.g., migratory plasmablasts, are found in the blood about 3 days to about 7 days (e.g., about 3 days, about 4 days, about 5 days, about 6 days, about 7 days) after antigen exposure. In some instances, ASCs e.g., migratory plasmablasts, are found in the blood about 8 days, about 9 days, or about 10 days after antigen exposure.

Suitable techniques for immunizing the non-human animal are known in the art. See, e.g., Goding, Monoclonal Antibodies: Principles and Practice, 3^{rd} ed., Academic Press Limited, San Diego, C A, 1996. The gene gun method described in, e.g., Barry et al., Biotechniques. 16(4):616-8, 620 (1994); Tang et al., Nature. 12; 356(6365):152-4 (1992); Bergmann-Leitner and Leitner, Methods Mol Biol 1325: 289-302 (2015); Aravindaram and Yang, Methods Mol Biol 542: 167-178 (2009); Johnston and Tang, Methods Cell Biol 43 PtA: 353-365 (1994); and Dileo et al., Human Gene Ther 14(1): 79-87 (2003), also may be used for immunizing the non-human animal. Furthermore, as exemplified herein, the immunizing may comprise administering cells expressing the antigen to the non-human animal or administering antigen-loaded dendritic cells, tumor cell vaccines, or immune-cell based vaccines. See, e.g., Sabado et al., Cell Res 27(1): 74-95 (2017), Bot et al., “Cancer Vaccines” in Plotkin's Vaccines. 7^{th} ed., Editors: Plotkin et al., Elsevier Inc., 2018, and Lee and Dy, “The Current Status of Immunotherapy in Thoraic Malignancies” in Immune Checkpoint Inhibitors in Cancer Editors: Ito and Ernstoff, Elsevier Inc., 2019. In various instances, the immunizing may be carried out by microneedle delivery (see, e.g., Song et al., Clin Vaccine Immunol 17(9): 1381-1389 (2010)); with virus-like particles (VLPs) (see, e.g., Temchura et al., Viruses 6(8): 3334-3347 (2014)); or by any means known in the art. See, e.g., Shakya et al., Vaccine 33(33): 4060-4064 (2015) and Cai et al., Vaccine 31(9): 1353-1356 (2013). Additional strategies for immunization and immunogen preparation, including, for example, adding T cell epitopes to antigens, are described in Chen and Murawsky, Front Immunol 9: 460 (2018).

In various aspects, the method comprises immunizing a non-human animal with an immunogen and said immunogen is administered to the non-human animal one or more (e.g., 2, 3, 4, 5, or more) times. In various aspects, the immunogens are administered by injection, e.g., intraperitoneal, subcutaneous, intramuscular, intradermal, or intravenous. In various aspects, the method comprises immunizing a non-human animal by administering a series of injections of the immunogen. In exemplary aspects, each administration, e.g., injection, is given to the non-human animal about 10 days to about 18 days apart, optionally, about 12 to about 16 days apart, or about 14 days apart. In exemplary aspects, each administration, e.g., injection, is given to the non-human animal more frequently than about 10 days to about 18 days apart. For instance, in exemplary aspects, the timing between administration of the immunogen to the non-human animal is about 1 to about 9 days apart, optionally, about 1 day to about 8 days, about 1 day to about 7 days, about 1 day to about 6 days, about 1 day to about 5 days, about 1 day to about 4 days, about 1 day to about 3 days, about 1 day to about 2 days, about 2 days to about 9 days, about 3 days to about 9 days, about 4 days to about 9 days, about 5 days to about 9 days, about 6 days to about 9 days, about 7 days to about 9 days, about 8 days to about 9 days, about 4 to about 8 days, about 4 days to about 8 days, or about 6 days to about 8 days. The timing between administration of the immunogen to the non-human animal may, in various aspects, be longer. For instance, the timing between administration of the immunogen to the non-human animal may be about 1 to about 20 weeks or longer, e.g., about 1 to about 20 months. Optionally, the timing between administration of the immunogen to the non-human animal is about 1 week to about 19 weeks, about 1 week to about 18 weeks, about 1 week to about 17 weeks, about 1 week to about 16 weeks, about 1 week to about 15 weeks, about 1 week to about 14 weeks, about 1 week to about 13 weeks, about 1 week to about 12 weeks, about 1 week to about 11 weeks, about 1 week to about 10 weeks, about 1 week to about 9 weeks, about 1 week to about 8 weeks, about 1 week to about 7 weeks, about 1 week to about 6 weeks, about 1 week to about 5 weeks, about 1 week to about 4 weeks, about 1 week to about 3 weeks, about 1 week to about 2 weeks, about 2 weeks to about 20 weeks, about 3 weeks to about 20 weeks, about 4 weeks to about 20 weeks, about 5 weeks to about 20 weeks, about 6 weeks to about 20 weeks, about 7 weeks to about 20 weeks, about 8 weeks to about 20 weeks, about 9 weeks to about 20 weeks, about 10 weeks to about 20 weeks, about 11 weeks to about 20 weeks, about 12 weeks to about 20 weeks, about 13 weeks to about 20 weeks, about 14 weeks to about 20 weeks, about 15 weeks to about 20 weeks, about 16 weeks to about 20 weeks, about 17 weeks to about 20 weeks, about 18 weeks to about 20 weeks, or about 19 weeks to about 20 weeks. In various aspects, the timing between administration of the immunogen may be longer than 8 or 9 days. Optionally, the timing between administration of the immunogen is about 1 month to about 8 months, about 1 month to about 7 months, about 1 month to about 6 months, about 1 month to about 5 months, about 1 month to about 4 months, about 1 month to about 3 months, about 1 month to about 2 months, about 2 months to about 9 months, about 3 months to about 9 months, about 4 months to about 9 months, about 5 months to about 9 months, about 6 months to about 9 months, about 7 months to about 9 months, about 8 months to about 9 months, about 4 to about 8 months, about 4 months to about 8 months, or about 6 months to about 8 months.

In various instances, during the immunization, each administration (e.g., injection) of immunogen is carried out with the same (A) immunogen, adjuvant, immunomodulatory agent, or combination thereof, (B) amount or dose of immunogen, adjuvant, immunomodulatory agent, or combination thereof, (C) administration route or method of delivering the immunogen, (D) administration site on the non-human animal, or (E) a combination thereof. Alternatively, one or more administrations (e.g., injections) of immunogen during the immunization is performed with a different (A) immunogen, adjuvant, immunomodulatory agent, or combination thereof, (B) amount or dose of immunogen, adjuvant, immunomodulatory agent, or combination thereof, (C) administration route or method of delivering the immunogen, (D) administration site on the non-human animal, or (E) a combination thereof. Optionally, the amount of immunogen decreases or increases with subsequent administrations, e.g., injections. In some aspects, every other administration, e.g., injection, comprises a decreased or increased amount of immunogen, relative to the first and third injections. Exemplary immunizations are described in the examples provided herein.

Non-Human Animals

Advantageously, the presently disclosed methods are not limited to any particular non-human animal. The non-human animal in exemplary aspects, is any non-human mammal. In exemplary aspects, the non-human animal is a mammal, including, but not limited to, mammals of the order Rodentia, such as mice, rats, guinea pigs, gerbils and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the non-human mammal is of the order Primates, Ceboids, or Simoids (monkeys) or of the order Anthropoids (apes). In various aspects, the non-human animal is a goat, llama, alpaca, chicken, duck, fish (e.g., salmon), sheep, or ram.

In exemplary instances, the non-human animal(s) used in the presently disclosed methods are modified, e.g., genetically modified, such that they produce chimeric or fully human antibodies. Such non-human animals are referred to as transgenic animals. The production of human antibodies in transgenic animals is described in Bruggemann et al., Arch Immunol Ther Exp (Warsz) 63(2): 101-108 (2015). Any transgenic animal can be use in the present invention including, but not limited to, transgenic chickens (e.g., OmniChicken®), transgenic rats (e.g., OmniRat®), transgenic llamas, and transgenic cows (e.g., Tc Bovine™). In a particular embodiment, the non-human animal is transgenic mouse such as XenoMouse®, Alloy mouse, Trianni mouse, OmniMouse®, and HuMAb-Mouse®. XenoMouse® is a strain of transgenic mice that produce full-human antibodies. An overview of XenoMouse® is provided by Foltz et al., Immunol Rev 270(1): 51-64 (2016) and U.S. Pat. No. 5,939,598. In exemplary aspects, the non-human animal is a transgenic rat. The transgenic rat in various aspects is Unirat® or OmniFlic®, which is described in Clarke et al., Front Immunol 9:3037 (2019); doi: 10.3389/fimmu.2018.03037 and Harris et al., Front Immunol 9:889 (2018): doi: 10.3389/fimmu.2018.00889, respectively.

In exemplary instances, the methods of the present disclosure are non-terminal with regard to the non-human animal. As used herein, the term “non-terminal” in the context of a non-human animal means that the life of the non-human animal is not terminated (e.g., not euthanized or otherwise killed or sacrificed) whilst the method is carried out. In exemplary aspects, the non-human animal is subjected to neither removal of one or more secondary lymphoid organs nor euthanasia, though the present invention does allow for procedures, such as biopsies and the like, of such organs (e.g., the spleen).

Immunogens

Advantageously, the presently disclosed methods are not limited to any particular immunogen. The immunogen in various aspects may be any antigen, optionally, a protein, or a fragment, fusion, or variant thereof. In various instances, the immunogen is a cytokine, lymphokine, hormone, growth factor, extracellular matrix protein, tumor associated antigen, tumor associated antigen, checkpoint inhibitor molecule, cell surface receptor, or a ligand thereof. For purposes of merely illustrating exemplary immunogens, the immunogen used in immunizing the non-human animal may be the target or antigen to which any one of the following antibodies bind: Muromonab-CD3 (product marketed with the brand name Orthoclone Okt3®), Abciximab (product marketed with the brand name Reopro®), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MbCampath®, Campath-1H®), Adalimumab (product marketed with the brand name Humira®), Tositumomab-I131 (product marketed with the brand name Bexxar®), Efalizumab (product marketed with the brand name Raptiva®), Cetuximab (product marketed with the brand name Erbitux®), Ibritumomab tiuxetan (product marketed with the brand name Zevalin®), Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®). In exemplary embodiments, the antibody is one of anti-TNF alpha antibodies such as adalimumab, infliximab, etanercept, golimumab, and certolizumab pegol; anti-IL10 antibodies such as canakinumab; anti-IL12/23 (p40) antibodies such as ustekinumab and briakinumab; and anti-IL2R antibodies, such as daclizumab.

Methods of preparing an immunogen for use in the immunization step are known in the art See, e.g., Fuller et al., CurrProtoc Mol Biol, Chapter 11, Unit 11.4, (2001); Monoclonal Antibodies: Methods and Protocols, 2^{nd} ed., Ossipow et al. (Eds.), Humana Press 2014. In various instances, the immunogen is mixed with an adjuvant or other solution prior to administration to the non-human animal. Many adjuvants are known in the art, and include, in exemplary instances, comprises an oil, an alum, aluminum salt, or a lipopolysaccharide. In various aspects, the adjuvant is inorganic. In alternative aspects, the adjuvant is organic. In various aspects, the adjuvant comprises: alum, aluminum salt (e.g., aluminum phosphate, aluminum hydroxide), Freund's complete adjuvant, Freund's incomplete adjuvant, RIBI adjuvant system (RAS), Lipid A, Sigma Adjuvant System®, TiterMax® Classic, TiterMax® Gold, a Montanide vaccine adjuvant (e.g., Montanide 103, Montanide ISA 720, Montanide incomplete Seppic adjuvant, Montanide ISA51), AF03 adjuvant, AS03 adjuvant, Specol, SPT, nanoemulsion, VSA3, oil or lipid-based solution, (e.g., squalene, MF59®, QS21, saponin, monophosphoryl lipid A (MPL)), trehalose dicorynomycolate (TDM), sTDM adjuvant, virosome, and PRR Ligands. See, e.g., “Vaccine Adjuvants Review” at https://www.invivogen.com/review-vaccine-adjuvants and “Role of Adjuvants in Antibody Production”, The Protein Man's Blog: A Discussion of Protein Research, posted on Jun. 2, 2016, at https://info.gbiosciences.com/blog/role-of-adjuvants-in-antibody-production. In various instances, the adjuvant comprises a surface-active substance such as lysolecithin, pluronic polyols, polyanions, peptides, oil emulsions, keyhole limpet hemocyanin, and dinitrophenol. BCG (bacilli Calmette-Guerin) and Corynebacterium parvum.

Blood Samples and Fractions Thereof

Following the immunization of the non-human animal, a blood sample comprising antibody secreting cells (ASCs) from said immunized non-human animal is obtained. ASCs are terminally differentiated cells of the humoral immune response; ASCs differentiate from activated B cells in lymph nodes and transiently circulate in the blood. In exemplary aspects, the blood sample is obtained from the non-human animal in a non-terminal manner, e.g., the non-human animal is not killed during the blood sample collection. In exemplary instance, the method comprises performing a non-terminal blood draw from the non-human animal. In exemplary aspects, the blood sample is obtained from the non-human animal about 1 to about 2 days after the non-human animal is immunized. In various instances, the blood sample is obtained from the non-human animal about 3 to about 7 days (e.g., 3, 4, 5, 6, or 7 days) post-immunization. If more than one administration of immunogen is given during the immunizing, the blood sample is obtained from the animal in some aspects about 3 day to about 7 days following the last administration of the immunogen. In various aspects, the blood sample is obtained from the non-human animal about 8 to about 12 days after immunizing the non-human animal, though, in some aspects, less ASCs are expected to be present in said blood sample.

In exemplary aspects, the blood sample comprises peripheral blood mononuclear cells (PBMCs). Optionally, the blood sample comprises B-lymphocytes, also known as B-cells. In various instances, the blood sample comprises ASCs of the plasma lineage, plasma cells and/or plasmablasts, e.g., migratory plasmablasts. In various aspects, the ASCs are CD138+ B cells. Optionally, the ASCs comprise migratory plasmablasts.

The volume of blood sample that can be taken depends on the non-human animal. In various instances, the blood sample obtained from the non-human animal is less than 1 L, 500 mL, or 100 mL, optionally, less than about 50 mL, less than about 25 mL, less than about 15 mL or 10 mL, less than or about 5 mL (e.g., about 4 mL, 3 mL, 2 mL, 1 mL or less). In some instances, the blood sample obtained from the non-human animal is about 1 L, 500 mL, or 100 mL, optionally, less than about 50 mL, less than about 25 mL, less than about 15 mL or 10 mL, less than or about 5 mL (e.g., about 4 mL, 3 mL, 2 mL, 1 mL or less). In some instances, 500 μL or less blood is obtained from the non-human animal. In embodiments where the non-human animal is a mouse, the blood sample obtained is less than 200 μL, 190 μL, 180 μL, 170 μL, 160 μL, 150 μL, 140 μL, 130 μL, 120 μL, 110 μL, 100 μL, 90 μL, 80 μL, 70 μL, 60 μL, 50 μL, 40 μL, 30 μL, 20 μL, 10 μL, 5 μL, 4 μL, 3 μL, 2 μL, or 1 μL. In other embodiments where the non-human animal is a mouse, the blood sample obtained is about 200 μL, 190 μL, 180 μL, 170 μL, 160 μL, 150 μL, 140 μL, 130 μL, 120 μL, 110 μL, 100 μL, 90 μL, 80 μL, 70 μL, 60 μL, 50 μL, 40 μL, 30 μL, 20 μL, 10 μL, 5 μL, 4 μL, 3 μL, 2 μL, or 1 μL. In exemplary instances, the blood sample obtained from the non-human animal is less than or about 500 μL. Optionally, the volume of the blood sample is about 100 μL to about 250 μL. In various instances, the volume of the blood sample is not more than 10% of the total amount of blood circulating in the animal. In various aspects, the volume of the blood sample does not exceed 10% of the blood volume circulating in the animal. In exemplary aspects, not more than about 10% of the total volume of the animal's blood is collected. In various instances, the volume of the blood sample is about 9% or less, about 8% or less, about 7% or less, about 6% or less, or about 5% or less of the blood volume circulating in the animal. In various instances, the blood sample represents not more than 10% of the animal's body weight. In various aspects, the blood sample is not more than 9%, not more than 8% or not more than 7% of the animal's body weight.

In exemplary aspects, after the blood sample is obtained from the non-human animal, the blood sample is processed, e.g., enriched or fractionated. In various instances, the method comprises enriching the blood sample for ASCs by, e.g., depleting red blood cells, plasma and/or platelets from the blood sample. In certain aspects, the method comprises a depletion step using an anti-IgM antibody to remove B-cells comprising a cell surface IgM. In exemplary instances, the method comprises a selection step in which cells expressing one or more cell surface markers which identify specific B-cell populations of interest is carried out. The cell surface marker is in some aspects CD138, CD19, B220, IgG, TACI, SLAM7, BCMA, CD98, SCA-1, Ly6C1/2, and the like. In instances where PBMC-derived B-cells are desired, the method comprises selecting for CD138− positive cells. In exemplary aspects, the method comprises removing one or more components of the blood sample obtained from the non-human animal prior to assaying. Optionally, red blood cells, plasma, and/or platelets are removed from the blood sample. In some aspects, a fraction of the blood sample is prepared by selecting for CD138+ cells.

Single-Cell Assays

In various aspects of the presently disclosed methods, ASCs present in the blood sample, or a fraction thereof, are individually assayed for the production of select antibodies. In various instances, the assaying comprises a single-cell assay in which one or more individual cells are analyzed. In various instances, the assaying comprises a live-cell assay in which one or more live cells are analyzed. In exemplary aspects, multiple cells, e.g., ASCs, present in the blood sample obtained from the immunized non-human animal are simultaneously assayed. In exemplary aspects, greater than about 10, greater than about 100, greater than about 500, greater than about 1000, greater than about 2000, greater than about 3000, greater than about 4000, greater than about 5000, greater than about 6000, greater than about 7000, greater than about 8000, greater than about 9000, or greater than about 10,000 ASCs are simultaneously assayed via a single-cell, live cell assay.

In various instances, the method comprises applying the blood sample, or a fraction thereof, to a matrix and assigning a unique address of the matrix to each ASC. The matrix may be two-dimensional wherein each unique address of the matrix is defined in terms of position along horizontal (X) and vertical (Y) axes, or the matrix is a three-dimensional matrix comprising, e.g., a porous foam, gel, or polymer, wherein each unique address of the matrix is defined in terms of position along width (X), height (Y), and depth (Z) axes. In various aspects, a result of the assaying is the identification of each ASC producing select antibodies, and, in certain aspects, the result is the identification of the unique address of each ASC producing select antibodies.

In exemplary aspects, the assaying of the presently disclosed methods comprises (a) combining the ASCs within the matrix with reagents that bind to the select antibodies and produce a detectable signal, e.g., a fluorescent signal, upon binding to the select antibodies. In various aspects, the assaying of the presently disclosed methods comprises (a) combining the ASCs within the matrix with at least one reagent which binds to the Fc domain of the select antibodies and at least one reagent to which select antibodies bind (e.g., a reagent which binds to the antigen-binding domain of the select antibodies), wherein at least one of these reagents is attached to a detectable label. In exemplary instances, the ASCs are combined with a detection reagent which binds to the Fc domain of the select antibodies and comprises a first detectable label and a target to which select antibodies bind (e.g., a reagent which binds to the antigen-binding domain of the select antibodies). In various instances, the target is expressed by cells and the cells expressing the target are combined with the ASCs and the detection reagent. In exemplary aspects, the method further comprises (b) assaying for the first detectable label; and (c) identifying the positions within the matrix at which the first detectable label is detected, wherein each identified position locates an individual ASC producing select antibodies.

In exemplary instances, the assaying of the presently disclosed methods comprises (a) combining the ASCs within the matrix with (i) a capture reagent which binds to the select antibodies and comprises a solid support, (ii) a detection reagent which binds to the select antibodies and comprises a first detectable label, and (iii) a labeled target to which the select antibodies bind, wherein the labeled target comprises a second detectable label distinct from the first detectable label; (b) assaying for the first detectable label and for the second detectable label; and (c) identifying the positions within the matrix at which both the first detectable label and the second detectable label are detected, wherein each identified position locates an individual ASC producing select antibodies. Optionally, the capture agent comprises an antibody that binds to an antibody Fc domain attached to a solid support. The solid support may be any solid supportive material, such as a polymer bead, a film, a slide, a well bottom, or the like, which anchors the anti-Fc domain antibody and the antibody to which the anti-Fc domain antibody binds. The detection agent, in exemplary instances, comprises an antibody that binds to an antibody Fc domain attached to a first detectable label. In various aspects, the antibody that binds to an antibody Fc domain of the capture agent is the same antibody of the detection agent, though the anti-Fc antibody of the capture reagent is not attached to a detectable label and the antibody of the detection reagent is not attached to a solid support.

In exemplary instances, the combining takes place in a well and the capture agent forms a monolayer in the well. In various aspects, the method comprises identifying the positions within the well at which both the first detectable label and the second detectable label are detected, wherein each identified position locates an individual ASC producing select antibodies.

In exemplary instances, the combining takes place in a microfluidic or nanofluidic chamber, a microwell or nanowell device, a microcapillary or nanocapillary tube, or a nanopen of a nanofluidic chip. In exemplary instances, the combining takes place in a nanopen of a nanofluidic chip. In exemplary instances, the method comprises identifying the position of each pen within the nanofluidic chip at which both the first detectable label and the second detectable label are detected, wherein each identified position locates an individual ASC producing select antibodies. Optionally, a single ASC of the blood sample is moved into a pen of the nanofluidic chip through optoelectro positioning (OEP). Such technique is described in Winters et al., MAbs 11(6): 1025-1035 (2016).

Antigen-specific B-cells that have recently encountered antigen in the germinal centers (GCs) of the secondary lymphoid organs (e.g., spleen and lymph nodes) are stimulated to divide and commit to differentiate down multiple pathways. The main B-cell lineage responsible for secreting antibodies into the serum in response to antigen challenge are plasma cells. Plasma cell differentiation begins in the secondary lymphoid organs where cell-cell interactions within the GCs force B-cells expressing antibodies on their surface that are specific to antigen to differentiate into immature plasma cells known as plasmablasts. Plasmablasts are rapidly dividing B-cells that produce and secrete high levels of soluble antibody. After exposure to antigen in the GCs, differentiation into plasmablasts and subsequent proliferation, a wave of migratory plasmablasts can be detected in circulation. In mice, plasmablasts in the blood occurs 3-7 days post antigen exposure and declines with time as they home to their appropriate niches and differentiate into long-lived plasma cells. The migration of recently stimulated, antigen-specific plasmablasts through the blood can be used to evaluate animal immune response and characteristics at the single-cell level rather than interrogation by polyclonal serum titer. In exemplary embodiments, non-terminal blood draws are collected from mice, washed to remove plasma and soluble antibodies, and peripheral blood mononuclear cells (PBMCs) assayed directly for ASCs. Small antibody capture beads are added to capture and localize secreted antibody from ASCs thereby enabling characterization at the single-cell level. Red blood cell (RBC) contaminants interfere with fluorescent plaque formation and can be mitigated by diluting the assay to a higher volume. However, this leads to a higher plating volume and lower assay throughput. Alternatively, RBCs can be directly removed or desired cells can be isolated from the blood sample before plating, thus, decreasing the plating volume and increasing the assay throughput. Suitable methods include, but are not limited to, RBC lysis, density gradient centrifugation (e.g., HetaSep, Ficoll®), and the use of negative selection (e.g., anti-mouse TER119 RBC depletion) or positive selection (e.g., Mouse CD138+ Isolation) cell separation kits, with or without the use of automatic washing instruments (e.g., Curiox Laminar Wash™). Cells expressing the target of interest can also be used instead of beads. Such techniques can be used to interrogate strategies for generating species reactive antibodies in mice.

In an exemplary embodiment, human-cyno cross-reactive antibodies can be produced by immunizing animals with alternating boosts of the human and cyno versions of the antigen. Reactivity to each antigen can be easily monitored using simple binding assays using the polyclonal serum from the immunized animals. However, since the animals have been immunized with both antigens, and the individual antigens harbor both common and unique epitopes, the polyclonal serum will contain antibodies reactive to both types of epitopes. Unfortunately, one cannot determine from this analysis alone, that the observed serum reactivity to both antigens is due to truly cross-reactive antibodies or derives from multiple antibodies with specificities for either antigen alone. However, interrogation of the B-cell response using the ASCs derived from the PBMC population overcomes this problem by localizing and screening the antibody specificity from that single cell which could then guide further immune repertoire shaping or animal selection for antibody discovery. Immune repertoire shaping can include modifications to the immunization strategy such as (but are not limited to) switching to different forms of the immunogen, adjuvants, immunomodulatory agents, doses of the antigen, timing of the immunizations and routes of administration.

Accordingly, the present disclosure additionally provides single-cell assays for identifying ASCs producing select antibodies. The present disclosure provides methods of assaying for ASCs producing select antibodies. In exemplary embodiments, the assay or method comprises (a) combining in a well (i) a blood sample obtained from a non-human animal immunized with an immunogen, or a fraction thereof, wherein the blood sample comprises ASCs, (ii) a detection reagent which binds to the select antibodies and comprises a first detectable label, and (iii) a target to which the select antibodies bind, wherein (A) the target is a labeled target comprising a second detectable label distinct from the first detectable label and a capture reagent which binds to the select antibodies and comprises a solid support is further combined in the well to form a monolayer in the well or (B) the target is expressed on the surface of cells and the cells are combined in the well to form a monolayer in the well; (b) assaying for the first detectable label and optionally assaying for the second detectable label, when the target is a labeled target; and (c) identifying the positions within the well at which the first detectable label is detected or the first and second detectable labels are detected, wherein each identified position locates an individual ASC producing select antibodies.

In various aspects, the assay or method comprises (a) combining in a well (i) a blood sample obtained from a non-human animal immunized with an immunogen, or a fraction thereof, (ii) a capture reagent comprising an antibody that binds to an Fc of an antibody attached to a solid support, (iii) a detection reagent comprising an antibody that binds to an Fc of an antibody attached to a first detectable label, and (iv) a labeled target comprising the immunogen, or a portion thereof, attached to a second detectable label distinct from the first detectable label, wherein the capture agent forms a monolayer in the well; (b) assaying for the first detectable label; (c) assaying for the second detectable label; and (d) identifying the positions within the well at which both the first detectable label and the second detectable label are detected, wherein each identified position locates an individual ASC producing select antibodies.

In various aspects, the assay or method comprises (a) combining in a well (i) a blood sample obtained from a non-human animal immunized with an immunogen, or a fraction thereof, (ii) a detection reagent which binds to the select antibodies and comprises a first detectable label, and (iii) cells expressing on the cell surface a target to which the select antibodies bind, wherein the cells are combined in the well to form a monolayer in the well; (b) assaying for the first detectable label; and (c) identifying the positions within the well at which the first detectable label are detected, wherein each identified position locates an individual ASC producing select antibodies.

In exemplary aspects, the first detectable label, the second detectable and/or label of the labeled target comprise(s) a chromophore or fluorophore. Optionally, the fluorophore comprises a xanthene derivative (e.g., fluorescein, rhodamine, Oregon green, eosin, and Texas red), a cyanine derivative (e.g., cyanine, indocarbocyanine, oxacarbocyanine, thiacarbocyanine, and merocyanine), a squaraine derivative (e.g., Seta and Square dyes), a squaraine rotaxane derivative (e.g., Tau dyes), a naphthalene derivative (e.g., dansyl and prodan derivatives), a coumarin derivative, an oxadiazole derivative (e.g., pyridyloxazole, nitrobenzoxadiazole and benzoxadiazole), an anthracene derivative (e.g., anthraquinones, including DRAQ5, DRAQ7 and CyTRAK Orange), a pyrene derivative (e.g., cascade blue), an oxazine derivative (e.g., Nile red, Nile blue, cresyl violet, oxazine 170), an acridine derivative (e.g., proflavin, acridine orange, acridine yellow), an arylmethine derivative (e.g., auramine, crystal violet, malachite green), a tetrapyrrole derivative (e.g., porphin, phthalocyanine, bilirubin), or a dipyrromethene derivative (e.g., BODIPY, aza-BODIPY). In various instances, the first detectable label, the second detectable and/or label of the labeled target comprises CF dye (Biotium), DRAQ or CyTRAK probe (BioStatus), BODIPY (Invitrogen), EverFluor (Setareh Biotech), Alexa Fluor (Invitrogen), Bella Fluor (Setareh Biotech), CyLight Fluor (Thermo Scientific, Pierce), Atto or Tracy (Sigma Aldrich), FluoProbe (Interchim), Abberior Dye (Abberior), DY or MegaStokes Dye (Dyomics), Sulfo Cy dye (Cyandye), HiLyte Fluor (AnaSpec), Seta, SeTau, Square Dye (SETA BioMedicals), Quasar or Cal Fluor dye (SETA BioMedicals), SureLight Dye (APC, RPEPerCP, Phycobiolisome (Columbia Biosciences), APC, APCXL, RPE, BPE (Phyco-Biotech, Greensea, Prozyme, Flogen), or a Vio Dyes (Miltenyi Biotec). In exemplary aspects, the fluorophore comprises 3-Hydroxyisonicotinaldehyde, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, G-Dye100, G-Dye200, G-Dye300, G-Dye400, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Texas Red, TRITC, TruRed, or X-Rhodamine. In various aspects, assaying for the first detectable label and/or the second detectable label comprises detecting a signal from the first detectable label and/or second detectable label. In exemplary instances, the signal is a fluorescent signal. In exemplary aspects, assaying for the first detectable label and/or the second detectable label comprises quantifying the signal from the first detectable label and/or second detectable label. In various instances, the method comprises quantifying the signal from the first detectable label and/or second detectable label and normalizing the signal by expressing the signal from the first detectable label and second detectable label as a ratio. In various aspects, the ratio is a relative fluorescence unit (RFU) of the signal from the first detectable label per RFU of the signal from the second detectable label, or the inverse thereof.

In exemplary aspects, the ASCs are first exposed to the detection reagent and/or target in the well or immediately prior to being added to the well. In various aspects, the ASCs are incubated with the detection reagent and the target for at least 30 minutes, at least 60 minutes, at least 90 minutes or at least 120 minutes. Optionally, the select antibodies bind to a target which is the same as or similar to the immunogen used to immunize the non-human animal. In exemplary instances, the detection reagent comprises an antibody that binds to an antibody Fc domain attached to a first detectable label. Optionally, the antibody that binds to an antibody Fc domain of the capture agent is the same antibody of the detection agent. In various aspects, the blood sample is obtained from the non-human animal about 3 to about 7 days after the immunizing step. In various instances, the blood sample obtained from the non-human animal is less than or about 500 μL, optionally, about 100 μL to about 250 μL. The ASCs are CD138+ B cells in certain aspects. Optionally, the ASCs comprise migratory plasmablasts. In exemplary aspects, the method further comprises removing one or more components of the blood sample obtained from the non-human animal prior to combining in the well. In some instances, red blood cells, plasma, and/or platelets are removed from the blood sample. The fraction of the blood sample is in various aspects prepared by selecting for CD138⁺ cells. In various instances, the select antibodies bind to the target in the presence of one or more competitive binding agents. Optionally, the competitive binding agents are combined with the ASCs, detection reagent, and cells expressing the target during the assaying. In exemplary aspects, the select antibodies bind to a target with a target affinity, optionally, wherein the KD of the select antibodies for the target is about 10⁻¹¹ M to about 10⁻⁹ M. Optionally, the assaying is carried out in a first round with a first amount of cells expressing the target and a second round with a second amount of the cells expressing the target, wherein the first amount is greater than the second amount. In some aspects, the assaying is further carried out in a third round with a third amount of the cells expressing the target and the third amount is less than the second amount, wherein when the ASC binds to the labeled target in each round, the ASC produces select antibodies.

The assaying of the presently disclosed methods test for the production of select antibodies by an individual ASC. The term “select antibodies” refers to antibodies that meet a design goal and/or exhibit a target phenotype. In various instances, the select antibodies bind to a target which may be the same as or similar to the immunogen used to immunize the non-human animal. The target may be any of the immunogens listed herein. In exemplary instances, the select antibodies are target-specific antibodies, e.g., antigen-specific antibodies. In various instances, the select antibodies exhibit a binding affinity for the target (or antigen) as represented by a KD of at least about 10⁻⁹ M. In various aspects, the select antibodies exhibit a KD in the picomolar range (e.g., a KD of about 1×10⁻¹² M to 9.9×10⁻¹² M. In various aspects, the select antibodies bind to the target in the presence of one or more competitive binding agents. In various instances, the competitive binding agents are components within human blood, e.g., human plasma or serum. In such instances, during the assaying, the competitive binding agents, e.g., human serum, are combined with the ASCs, capture reagent, detection reagent, and labeled target during the assaying. See, e.g., Example 1. In various aspects, the competitive binding agents are native ligands that bind to the target in a human or non-human animal body and the select antibodies binding to the target prevent or inhibit binding of the native ligand to the target. For instance, the select antibodies are anti-PD-1 antibodies and the competitive binding agents are PD-L1 and/or PD-L2. In such instances, during the assaying, the competitive binding agents, e.g., PD-L1 and/or PD-L2, are combined with the ASCs, capture reagent, detection reagent, and labeled target during the assaying. See, e.g., Example 7.

Screening of the disclosed method can be completed in larger wells (e.g., 4 well plate or OmniTmy™) for subsequent molecular rescue of ASCs producing select antibodies. Unlike ELISpot or FluoroSpot assays, the disclosed method is a homogenous live cell assay that is amendable to micromanipulation that is known in the art or with automated fluorescent single cell picking systems (e.g., CellCelector™). Confluent monolayer of IgG capture beads locks ASCs in place enabling well defined fluorescent plaques and identified position of individual ASC producing select antibodies.

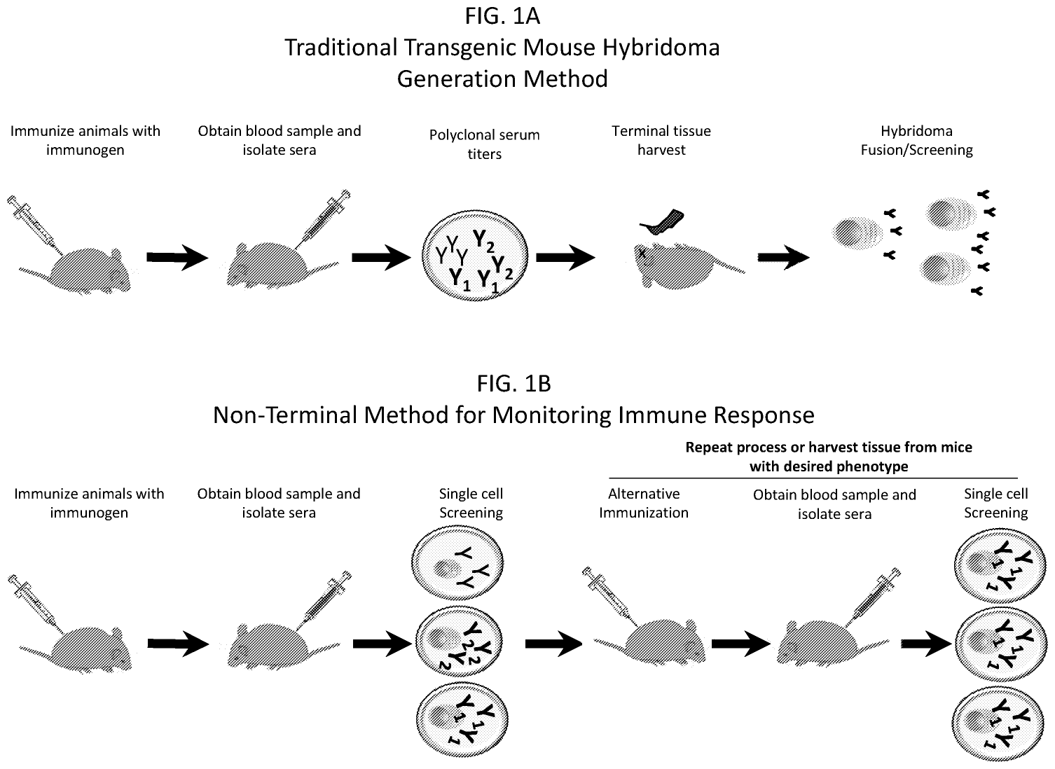

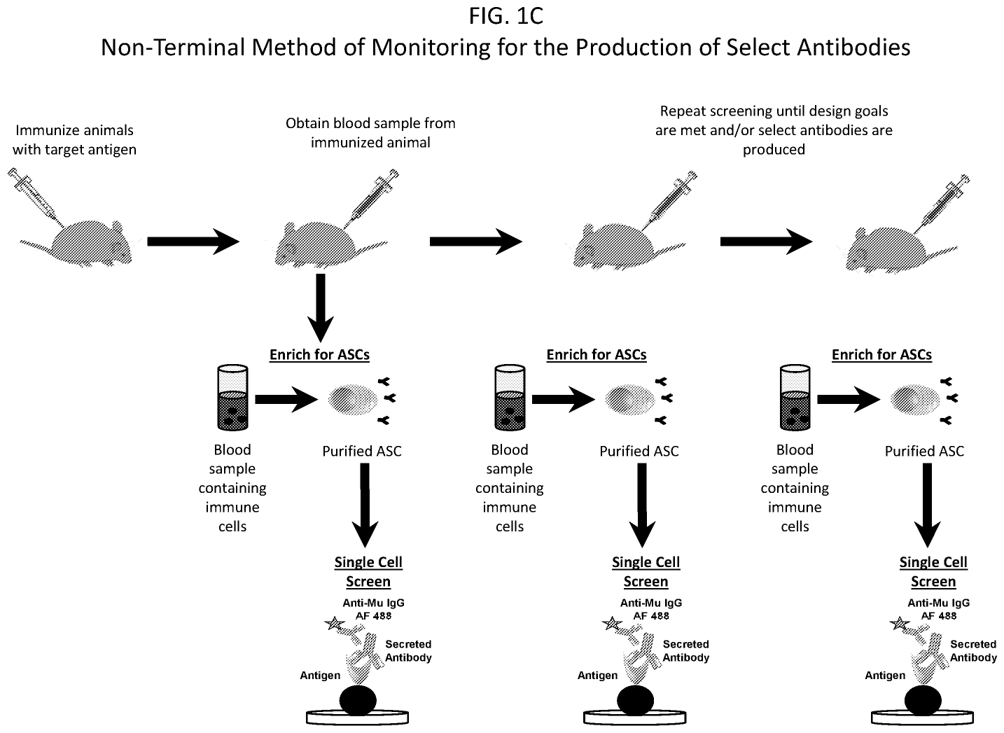

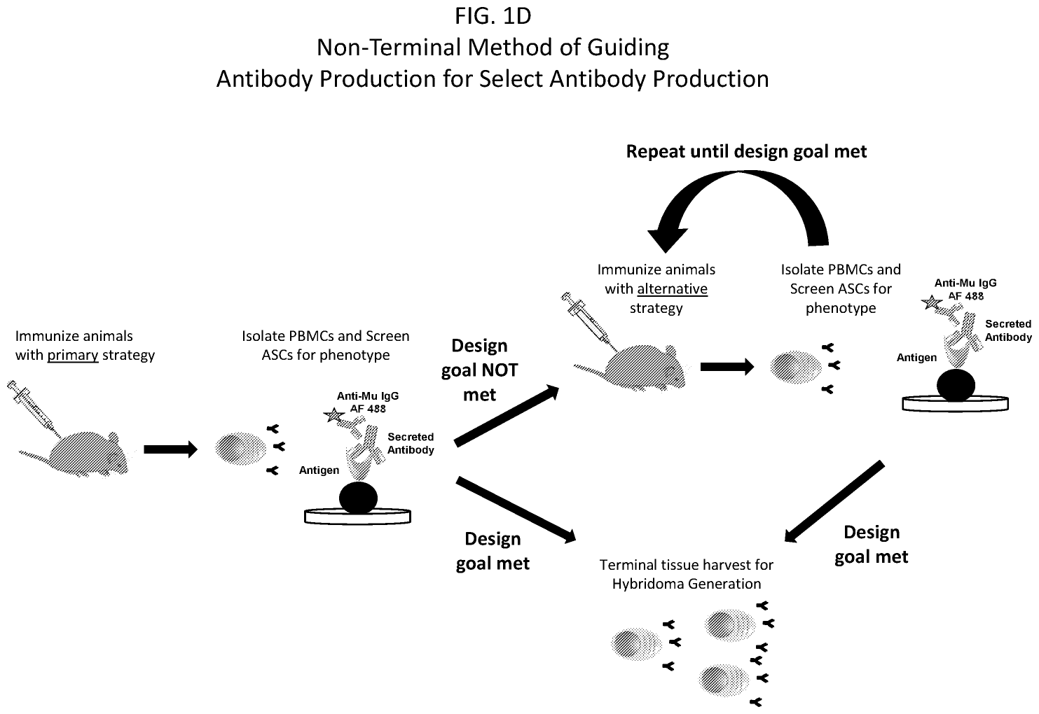

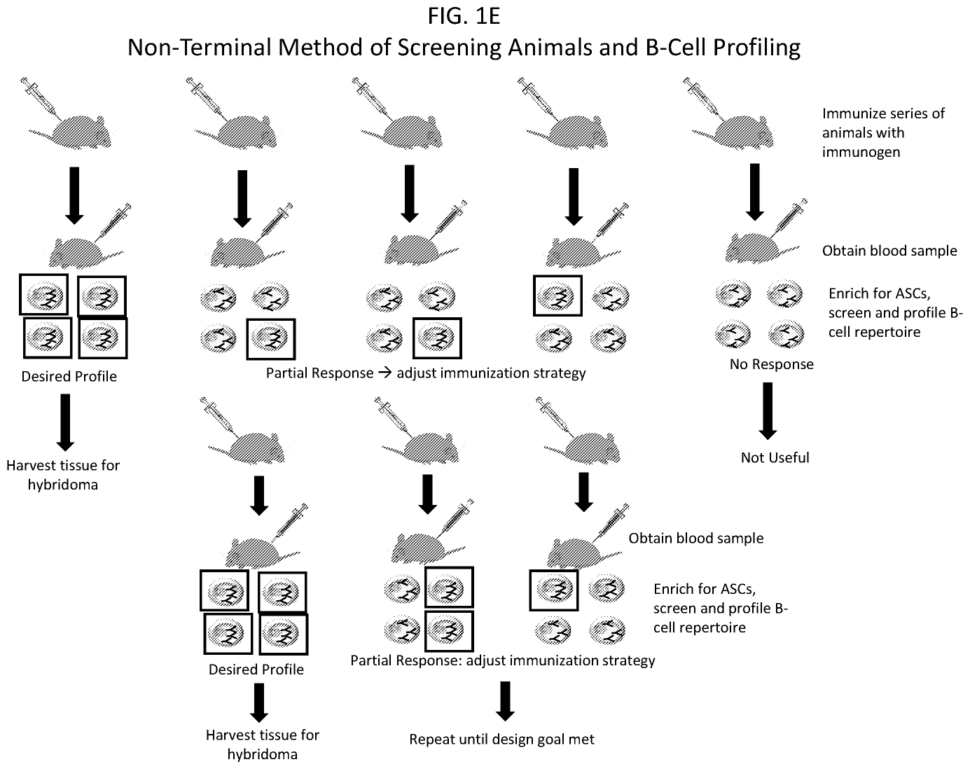

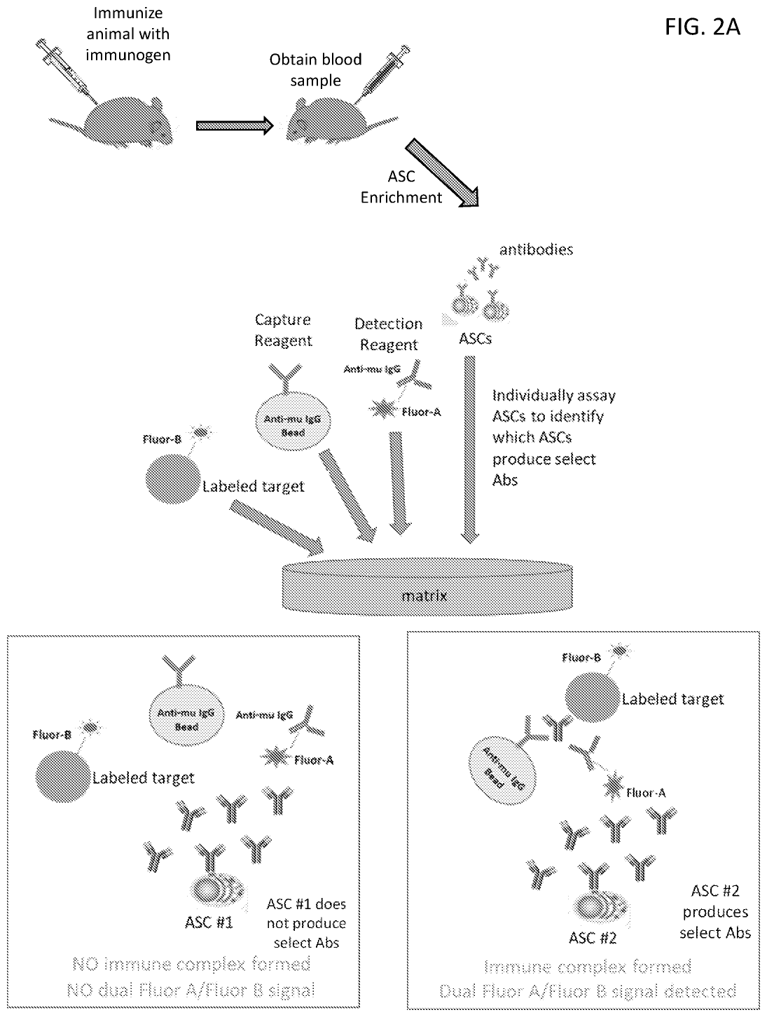

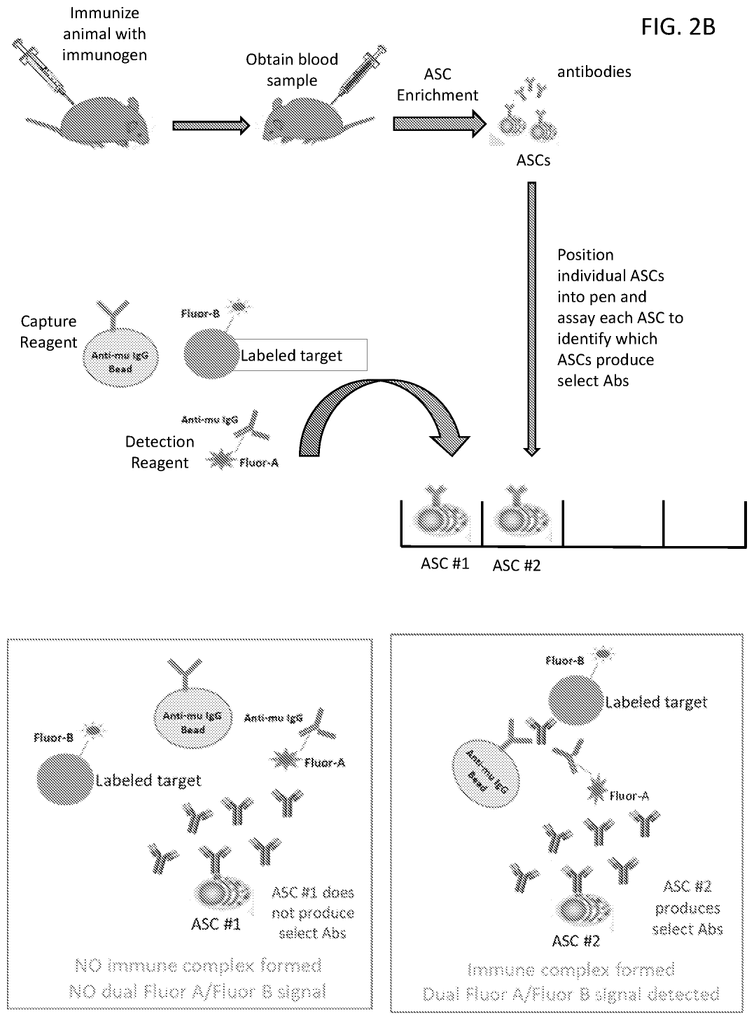

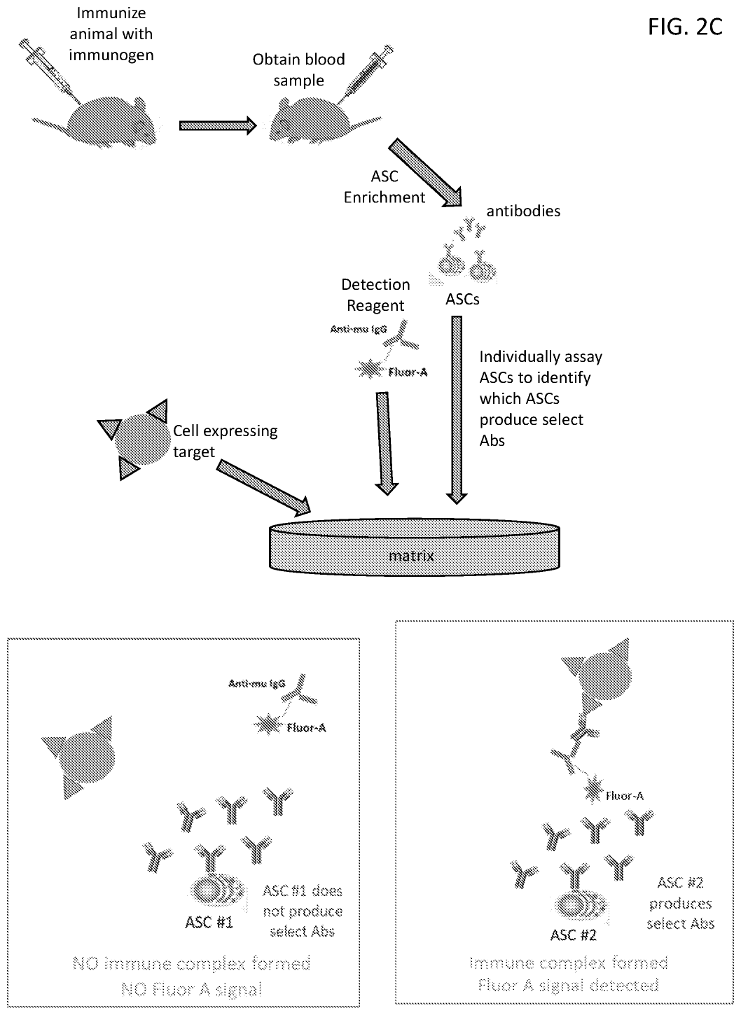

The disclosed method can guide selection and harvest of animals producing select antibodies for antibody discovery. Traditional approaches to select animals rely on interrogation of the polyclonal serum titer that measures the total reactivity and quality of all the secreted antibodies rather than the quality of the individual antibodies. Interrogation of the B-cell response using the ASCs derived from the PBMC population can overcome this problem by identifying individual ASCs producing select antibodies that would otherwise be difficult to interpret or would be hidden in the polyclonal serum titer. Exemplary methods of animal selection for terminal tissue harvest are described in FIGS. 1D and 1E and Examples 12 and 13.

In exemplary aspects, the select antibodies bind to the target with a target affinity, optionally, wherein the KD of the select antibodies for the target is within the range of 10⁻¹¹ M to 10⁻⁹ M. In various instances, the KD of the select antibodies for the target is within the picomolar range or about 10⁻¹² M. In various instances, the KD of the select antibodies for the target is sub-picomolar range, e.g., <10⁻¹² M. In various aspects, the assaying comprises combining in a first round a first amount of labeled target comprising the immunogen, or a portion thereof, attached to a second detectable label with the ASCs, capture reagent, and detection reagent, and in a second round, a second amount of labeled target comprising the immunogen, or a portion thereof, attached to a second detectable label is combined with the ASCs, capture reagent, and detection reagent, wherein the first amount is greater than the second amount. The assaying may comprise in some instances a third round using a third amount of labeled target, wherein the third amount is less than the second amount. Those ASCs that are identified by detection of the first detectable label and the second detectable label throughout each round may be ASCs producing select antibodies having a high affinity for the target. See, e.g., Example 8.

The select antibodies bind to the target with a target affinity. In various aspects, the assaying comprises combining ASCs with a detection reagent which binds to the Fc domain of the select antibodies and comprises a first detectable label and a target to which select antibodies bind as the second label. This is followed by fluorescence signal quantification and secretion normalization by determining the ratio of IgG secretion first label RFU (relative fluorescence unit) to the target second label RFU. Those ASCs identified with the smallest ratio (IgG RFU/Target RFU) may be ASCs producing select antibodies having a high affinity for the target. ASC IgG normalized RFUs can be compared to ASCs of known target affinities from validated hybridomas or recombinant antibodies expressed in cell lines to provide rough relative affinity ranking. See, e.g., Example 11.

In various aspects, the select antibodies bind to a target and to an ortholog or paralog thereof, optionally, wherein the target is a human protein and the ortholog is a cynomolgus monkey protein. In various instances, during the assaying, a second labeled target is combined with the ASCs, capture reagent, detection reagent, and labeled target, wherein the second labeled target comprises the ortholog attached to a third detectable label which is distinct from the first detectable label and the second detectable label, wherein the method further comprises assaying for the third detectable label and identifying the position(s) at which the first detectable label, the second detectable label, and the third detectable label are detected, wherein each identified position locates an individual ASC producing select antibodies.

In various instances, the select antibodies bind to a target and not to an ortholog or paralog thereof. Optionally, during the assaying, a second labeled target is combined with the ASCs, capture reagent, detection reagent, and labeled target, wherein the second labeled target comprises the ortholog attached to a third detectable label which is distinct from the first detectable label and the second detectable label, wherein the method further comprises assaying for the third detectable label and identifying the position(s) at which only the first detectable label and the second detectable label, but not the third detectable label, are detected, wherein each identified position locates an individual ASC producing select antibodies. See, e.g., Example 6.

In various aspects, the select antibodies bind to a portion of the target. Optionally, during the assaying, a second labeled target is combined with the ASCs, capture reagent, detection reagent, and labeled target, wherein the second labeled target comprises the portion of the target attached to a third detectable label which is distinct from the first detectable label and the second detectable label, and wherein the method further comprises assaying for the third detectable label and identifying the position(s) at which the first detectable label, the second detectable label, and the third detectable label are detected, wherein each identified position locates an individual ASC producing select antibodies. In various instances, the target is a protein comprising multiple domains and the select antibodies bind to only one domain of the target. In various aspects, during the assaying, the labeled target comprises the extracellular domain of the target attached to the second detectable label and the second labeled target comprises the one domain attached to a third detectable label. See, e.g., Example 9. In various aspects, the select antibodies bind to a conformational epitope formed upon dimerization or multimerization of the target and the target comprises a dimerization domain or multimerization domain. Optionally, during the assaying, the labeled target comprises the extracellular domain of the immunogen attached to the second detectable label, wherein a second labeled target is combined with the ASCs, capture reagent, detection reagent, and labeled target, wherein the second labeled target comprises the dimerization domain or multimerization domain of the immunogen attached to the third detectable label which is distinct from the first detectable label and the second detectable label, and wherein the method further comprises assaying for the third detectable label and identifying the position(s) at which the first detectable label, the second detectable label, and the third detectable label are detected, wherein each identified position locates an individual ASC producing select antibodies. See, e.g., Example 10.

Guiding Antibody Production by Repeated Immunizations

In various aspects, the method comprises repeatedly immunizing the non-human animal. As exemplified herein, in various aspects, the method comprises immunizing the non-human animal more than once. In various aspects, the method comprises performing an initial immunization and one or more subsequent immunizations. In various instances, each subsequent immunization is repeated after obtaining a blood sample from the non-human animal and assaying for ASCs producing select antibodies. In various aspects, the non-human animal is immunized at least two or more times, e.g., 2, 3, 4, 5, 6, 7, 8, 9, 10, 11, 12, 13, 14, 15, 16, 17, 18, 19, 20, or more times. In various aspects, the method comprises performing an initial immunization and 1, 2, 3, 4, 5, 6, 7, 8, 9, or 10 subsequent immunizations. Optionally, the immunizations are repeated until ASCs producing select antibodies are identified or a percentage of the ASCs producing select antibodies is at or above a threshold. In various instances, each immunization is repeated with the same (A) immunogen, adjuvant, immunomodulatory agent, or combination thereof, (B) amount or dose of immunogen, adjuvant, immunomodulatory agent, or combination thereof, (C) administration timing, (D) administration route or method of delivering the immunogen, (E) administration site on the non-human animal, or (F) a combination thereof, compared to the prior immunization or initial immunization. Alternatively, each immunization is repeated with a different (A) immunogen, adjuvant, immunomodulatory agent, or combination thereof, (B) amount or dose of immunogen, adjuvant, immunomodulatory agent, or combination thereof, (C) administration timing, (D) administration route or method of delivering the immunogen, (E) administration site on the non-human animal, or (F) a combination thereof, compared to the prior immunization or initial immunization. In exemplary aspects, for each time the animal is immunized, a different (A) immunogen, adjuvant, immunomodulatory agent, or combination thereof, (B) amount or dose of immunogen, adjuvant, immunomodulatory agent, or combination thereof, (C) administration timing, (D) administration route or method of delivering the immunogen, (E) administration site on the non-human animal, or (F) a combination thereof, compared to the prior immunization or initial immunization, is used. In various aspects, the immunizing changes with each occurrence so that the immune response elicited thereby in the non-human animal is modified, relative to the prior immune response caused by the prior immunizing. Without being bound to any particular theory, performing multiple different immunization campaigns on the same animal guides the immune response toward the production of select antibodies.

In exemplary instances, the method of guiding antibody production in a non-human animal for the production of select antibodies comprises performing a cycle of steps when the percentage of ASCs producing select antibodies is below a threshold, wherein the cycle comprises (i) performing a subsequent immunization on the non-human animal with an immunogen when the percentage of ASCs producing select antibodies is below a threshold, (ii) obtaining a blood sample comprising ASCs from said non-human animal, and (iii) individually assaying ASCs present in the blood sample, or a fraction thereof, for the production of select antibodies.

In exemplary instances, the method comprises a cycle of (i) performing a subsequent immunization on the non-human animal with an immunogen when the percentage of ASCs producing select antibodies is below a threshold, (ii) obtaining a blood sample comprising ASCs from said non-human animal, (iii) individually assaying ASCs present in the blood sample, or a fraction thereof, for the production of select antibodies. In various instances, the cycle is repeated at least 1, 2, or 3 or more times. In various aspects, the cycle is repeated until the number of ASCs producing select antibodies, as assayed in (iii), is at or above the threshold. In exemplary aspects, after the repeated cycles, greater than 10%, 20%, 30%, 40%, or 50% of the immunized non-human animals yield a percentage of ASCs producing select antibodies which is at or above a threshold. In various aspects, greater than 75% or greater than 85% or greater than 90% of the immunized non-human animals yield a percentage of ASCs producing select antibodies which is at or above a threshold. In exemplary aspects, the immunogen of the subsequent immunization is different from the immunogen of the initial immunization. For instance, in exemplary aspects, each subsequent immunization differs from a prior immunization in that (A) a different immunogen, adjuvant, and/or immunomodulatory agent is administered to the non-human animal, (B) a different dose of the immunogen of the initial immunization is administered to the non-human animal, (C) the time between each administration of the immunogen, adjuvant, and/or immunomodulatory agent used in the initial immunization is different, and/or (D) the route of administration for each administration of immunogen, adjuvant, and/or immunomodulatory agent used in the initial immunization is different. Optionally, a different immunogen is used each time the non-human animal is immunized.

As discussed herein, the immunizing comprises one or more administrations of the immunogen (optionally prepared with an adjuvant) to the non-human animal. The methods of the present invention may comprise multiple immunization steps—with differing immunization conditions—which can be used to steer the immune response such that the immunized non-human animal eventually generates antibodies with desired phenotypes. Depending on the phenotype, or combination of phenotypes, of interest, the immunization conditions can be varied during successive immunization steps such that the immune response is steered to generating antibodies with desired phenotypes. In exemplary embodiments, to produce human-cyno cross-reactive antibodies, non-human animals may be immunized with alternating boosts of the human and cyno versions of the antigen. For instance, the immunization may comprise a total of four injections: for the first and third injections, a recombinant human antigen may be used and for the second and fourth injections, recombinant cynomolgus monkey antigen may be used. An exemplary immunization is described herein in Example 4. Example 5 describes an additional method of making human-cyno cross-reactive antibodies, wherein a different immunization is used. In exemplary embodiments, to produce antibodies specific for a domain of a multi-domain protein, immunization may occur with one or more of three types of immunogens: the full extracellular domain of the multi-domain protein, the domain, and/or the full-length protein. See, e.g., Example 9. In exemplary embodiments, to produce antibodies specific for an epitope that forms upon dimerization or multimerization of a dimeric or multimeric protein, immunization may occur with one or more of the three types of immunogens: the full extracellular domain of the dimeric or multimeric protein, the multimerization domain and/or the full-length protein. See, e.g., Example 10. Additional exemplary immunizations are provided herein. See, EXAMPLES.

Additional Steps

The methods disclosed herein may comprise additional steps. In exemplary instances, the method comprises assaying for an antibody response against the immunogen after the blood sample is obtained. In exemplary instances, after the blood sample is obtained, the method comprises assaying the antibody titer of the sample. In exemplary instances, the method comprises assaying the antigen-specificity of the antibodies present in the blood sample, optionally, a binding assay using the immunogen.

In various aspects, the method comprises isolating ASCs producing select antibodies or isolating the select antibodies. In various instances, the isolating of antibodies is achieved by isolating a single ASC producing select antibodies. In various aspects, isolating an ASC comprises a dilution step, optionally, a serial dilution step, wherein the cell concentration decreases so that, statistically, one cell is present in a given calculated volume, which calculated volume is placed into a separate container or well of a multi-well plate. In various aspects, isolating an ASC of the blood sample comprises microfluidically moving a single ASC into a well or into a bubble. There, the ASC is maintained in culture until select antibodies are secreted into the culture medium and/or the ASC undergoes cell division. Optionally, the maintaining occurs for at least or about 3 minutes to about 30 minutes, 6 hours, 24 hours, or longer. In various aspects, the isolating of the ASC occurs via microfluidics, magnetism, capillary action, gravity, FACS or optoelectro positioning (OEP).

In various aspects, the methods of the present disclosure comprise sequencing the heavy chain variable region and light chain variable region of the antibodies having the target phenotype. Optionally, the sequencing is carried out via RT-PCR. Optionally, the method further comprises transfecting cells with nucleic acids encoding the heavy chain variable region and light chain variable region of the antibodies having the target phenotype; culturing the transfected cells; and harvesting antibodies from the culture. In some aspects, the steps of the method are carried out on a series of non-human animals and the method comprises profiling the B-cell repertoire of the blood sample for each non-human animal of the series and selecting a subset of the series having a target B-cell profile. Such methods are described in Example 1.

Also, in various aspects, the methods comprise one or more upstream steps or downstream steps involved in producing, purifying, and formulating an antibody. Optionally, the downstream steps are any one of those downstream processing steps described herein or known in the art. In exemplary embodiments, the method comprises steps for generating host cells that express the antibody with the target phenotype. The host cells, in some aspects, are prokaryotic host cells, e.g., E. coli or Bacillus subtilis, or the host cells, in some aspects, are eukaryotic host cells, e.g., yeast cells, filamentous fungi cells, protozoa cells, insect cells, or mammalian cells (e.g., CHO cells). Such host cells are described in the art. See, e.g., Frenzel, et al., Front Immunol 4: 217 (2013). For example, the methods comprise, in some instances, introducing into host cells a vector comprising a nucleic acid comprising a nucleotide sequence encoding the antibody, or a light chain or heavy chain thereof. In exemplary aspects, the methods comprise maintaining cells in a cell culture. Optionally, such step may include maintaining a particular temperature, pH, osmolality, dissolved oxygen, humidity, or in a culture medium comprising one or more of glucose, fucose, lactate, ammonia, glutamine, and/or glutamate.

In exemplary embodiments, the methods disclosed herein comprise steps for isolating and/or purifying the ASC producing select antibodies or isolating and/or purifying the select antibodies from the culture. In exemplary aspects, the method comprises one or more chromatography steps including, but not limited to, e.g., affinity chromatography (e.g., protein A affinity chromatography), ion exchange chromatography, and/or hydrophobic interaction chromatography. In exemplary aspects, the method comprises steps for producing crystalline biomolecules from a solution comprising the recombinant glycosylated proteins.

The methods of the disclosure, in various aspects, comprise one or more steps for preparing a composition, including, in some aspects, a pharmaceutical composition, comprising the purified select antibodies.

In exemplary embodiments, the method comprises (a) immunizing animals using standard protocols and (b) evaluating sera for antigen-specific antibody responses. In exemplary aspects, the method further comprises selecting immune animals for a boost with antigen and harvesting blood from these animals (e.g., about 4 days later after the boost). In various instances, the method comprises removing red blood cells, plasma, and platelets from the blood collected from the animals to enrich the blood for B-cells. In exemplary instances, the method comprises identifying antibody secreting cells (ASCs) and isolating the ASCs as single cells to allow for elucidation and/or characterization of the antibody produced by an individual ASC. Optionally, single cell isolation and screening are accomplished using approaches known in the art, e.g., NanOBLAST (e.g., on a nanofluidic Beacon device), microencapsulation. In various aspect, the antibody produced by and secreted from the individual ASC is evaluated for a target phenotype. Optionally, the evaluation for the target phenotype is accomplished by using a variety of different screening strategies, and one or more antibodies having the target phenotype, as well as the ASC that produces and secretes the antibodies, are identified. In various instances, the method further comprises isolating from the ASCs (which produce and secrete the antibodies exhibiting the target phenotype) antibody VH and VL genes by, e.g., single cell RT-PCR, and cloning sequences of the paired VH and VL genes into cells for recombinant production.

Screening, Selection, and Profiling Methods

The present disclosure provides a method of screening non-human animals for antibody secreting cells (ASCs) producing select antibodies. In exemplary embodiments, the method comprises monitoring for the production of select antibodies in a non-human animal in accordance with the presently disclosed methods of monitoring, wherein the method is carried out on a series of non-human animals, wherein for each non-human animal of the series the number of ASCs producing the select antibodies is identified. In exemplary embodiments, the method comprises (a) immunizing a series of non-human animals with an immunogen; (b) obtaining a blood sample comprising ASCs from each non-human animal of the series; and (c) individually assaying ASCs present in the blood sample, or a fraction thereof, for the production of select antibodies, wherein, for each non-human animal of the series, a percentage of ASCs producing select antibodies is determined. In various aspects, the screening method further comprises selecting the non-human animal(s) for sacrifice and/or tissue harvest, e.g., secondary lymphoid tissue harvest, when the percentage of ASCs producing select antibodies is at or above a threshold. The present disclosure further provides methods of selecting immunized non-human animals for subsequent immunization. In various aspects, the screening method further comprises selecting the non-human animal(s) for subsequent immunization, when the percentage of ASCs producing select antibodies is below a threshold. According, in various embodiments, the screening method identifies animals for sacrifice and animals for subsequent immunization based on the percentage of ASCs producing select antibodies. In exemplary embodiments, the method comprises monitoring for the production of select antibodies in a non-human animal in accordance with the presently disclosed methods of monitoring, wherein the method is carried out on a series of non-human animals, wherein for each non-human animal of the series the number of ASCs producing the select antibodies is identified, and selecting the animal for subsequent immunization when the percentage of ASCs producing select antibodies for an animal is below a threshold. Also further provided herein are methods of selecting immunized non-human animals producing select antibodies for euthanasia and secondary lymphoid harvest. In exemplary embodiments, the method comprises monitoring for the production of select antibodies in a non-human animal in accordance with the presently disclosed methods of monitoring, wherein the method is carried out on a series of non-human animals, wherein for each non-human animal of the series the number of ASCs producing the select antibodies is identified, and selecting the animal for euthanasia and secondary lymphoid harvest, when the percentage of ASCs producing select antibodies for an animal is at or above a threshold.

The present disclosure further provides methods of profiling the B-cell repertoire of a non-human animal. In exemplary embodiments, the method comprises (a) immunizing a non-human animal with an immunogen; (b) obtaining a blood sample comprising antibody secreting cells (ASCs) from said non-human animal; and (c) individually assaying ASCs present in the blood sample, or a fraction thereof, for the production of select antibodies. In various instances, the method is carried out on a series of non-human animals and the method comprises profiling the B-cell repertoire of the blood sample for each non-human animal of the series and selecting a subset of the series having a target B-cell profile. In exemplary instances, the subset is selected for re-immunization. In alternative instances, the subset is selected for euthanasia and harvesting for secondary lymphoid organs.

Accordingly, the screening and selection methods described herein allow for the identification of non-human animals that are producing select antibodies. An exemplary benefit of such methods is that non-human animals producing such antibodies can be identified before sacrifice and B cell harvesting. This enriches the non-human animals, and thus the B cell pools, for those producing select antibodies, thus helping to mitigate some of the inefficiencies of traditional downstream antibody discovery methods.

Antibody Production

The present disclosure further provides methods of producing select antibodies in a non-human animal. In exemplary embodiments, the method comprises guiding antibody production in a non-human animal for the production of select antibodies in accordance with the presently disclosed methods of guiding antibody production and then isolating the select antibodies and/or an ASC producing the select antibodies. In exemplary embodiments, the method comprises (a) performing an initial immunization campaign on a non-human animal with an immunogen; (b) obtaining a blood sample comprising antibody secreting cells (ASCs) from said non-human animal; (c) individually assaying ASCs present in the blood sample, or a fraction thereof, for the production of select antibodies; (d) performing a subsequent immunization on the non-human animal with an immunogen when the percentage of ASCs producing select antibodies is below a threshold; and (e) isolating the select antibodies and/or an ASC producing the select antibodies. In various aspects, the method comprises repeating a cycle of (i) performing a subsequent immunization on the non-human animal with an immunogen when the percentage of ASCs producing select antibodies is below a threshold, (ii) obtaining a blood sample comprising ASCs from said non-human animal, (iii) individually assaying ASCs present in the blood sample, or a fraction thereof, for the production of select antibodies, until the percentage of ASCs producing select antibodies is at or above a threshold. Methods of isolating ASCs producing select antibodies or isolating the select antibodies are described herein. See, e.g., Additional Steps.

In various aspects, the method further comprises (f) determining the nucleotide sequence encoding the heavy chain variable region of the select antibodies produced by an ASC (e.g., the isolated ASC producing the select antibodies) and the nucleotide sequence encoding the light chain variable region of the select antibodies produced by the ASC, (g) introducing into a host cell a first vector comprising the nucleotide sequence encoding the heavy chain variable region of the select antibodies and a second vector comprising the nucleotide sequence encoding the light chain variable region of the select antibodies, and (h) isolating the antibodies produced by the host cell. Methods of determining the sequences of the heavy chain and light chain variable regions of antibodies are known in the art, and include, for instance single-cell PCR. See, e.g., Tiller et al., J Immunol Methods 350: 189-193 (2009); and Winters et al., 2019, supra. Generating vectors comprising the nucleotide sequences are known. See, e.g., Green and Sambrook, Molecular Cloning: A Laboratory Manual, Cold Spring Harbor Laboratory Press, 2012. In various aspects, the method of producing select antibodies comprises engineering the heavy chain sequence and/or the light chain sequence to achieve an engineered select antibody. In various aspects, the engineered select antibody exhibits higher stability, e.g., during storage or manufacture, formulation, filling, transportation or administration or under in vivo conditions, compared to the non-engineered select antibody. In various aspects, the engineered select antibody exhibits higher affinity for the target or an ortholog or paralog thereof, compared to the non-engineered select antibody. Suitable techniques for isolating the antibodies produced by the host cells are described herein and known in the art. See, e.g., Additional Steps herein, and Low et al., J Chromatog B 848(1): 48-63 (2007); Ngo et al., U.S. Pat. No. 4,933,435; and Ayyar et al., Methods 56(2): 116-129 (2012).

In exemplary embodiments of the presently disclosed methods of producing select antibodies, the method comprises (a) performing an initial immunization on a non-human animal with an immunogen; (b) obtaining a blood sample comprising antibody secreting cells (ASCs) from said non-human animal; (c) individually assaying ASCs present in the blood sample, or a fraction thereof, for the production of select antibodies; and (d) harvesting one or more secondary lymphoid organs from the non-human animal when the percentage of ASCs producing select antibodies is at or above a threshold. In various aspects, immune cells from the harvested secondary lymphoid organ(s) are obtained and at least a portion of those immune cells, e.g., IgG-positive memory B cells, are used to generate hybridomas. Methods of generating hybridomas are known in the art and described herein. See, e.g., Enhanced Hybridoma Generation herein and Zhang, Methods Mol Ciol 01: 117-135 (2012); Tomita and Tsumoto, Immunotherapy 3(3): 371-380 (2011); and Hnasko and Stanker, Methods Mol Biol 1318: 15-28 (2015) and Zaroff and Tan, Biotechniques 67(3): 90-92 (2019). The presently disclosed methods in certain aspects further comprise generating a hybridoma.

Antibodies

Although antibody structures vary between species, as used herein, the term “antibody” generally refers to a protein having a conventional immunoglobulin format, typically comprising heavy and light chains, and comprising variable and constant regions. Antibodies obtained or isolated by the present method can have a variety of uses. For example, antibodies obtained by the present method can be used as therapeutics. The antibodies obtained by the present method can also be used as non-therapeutic antibodies as, for example, reagents used in diagnostic assays, e.g., diagnostic imaging assays, and for other in vitro or in vivo immunoassays, e.g., Western blots, radioimmunassays, ELISA, EliSpot assay, and the like. In various aspects, the antibody can be a monoclonal antibody or a polyclonal antibody. In exemplary instances, the antibody is a mammalian antibody, e.g., a mouse antibody, rat antibody, rabbit antibody, goat antibody, horse antibody, chicken antibody, hamster antibody, pig antibody, human antibody, alpaca antibody, camel antibody, llama antibody, and the like. In some aspects, the antibody can be a monoclonal antibody or polyclonal antibodies optionally produced by a transgenic animal. In such embodiments, the antibodies produced are chimeric antibodies comprising sequences of two or more species. In various instances, an antibody has a human IgG which is a “Y-shaped” structure of two identical pairs of polypeptide chains, each pair having one “light” (typically having a molecular weight of about 25 kDa) and one “heavy” chain (typically having a molecular weight of about 50-70 kDa). A human antibody has a variable region and a constant region. In human IgG formats, the variable region is generally about 100-110 or more amino acids, comprises three complementarity determining regions (CDRs), is primarily responsible for antigen recognition, and substantially varies among other antibodies that bind to different antigens. See, e.g., Janeway et al., “Structure of the Antibody Molecule and the Immunoglobulin Genes”, Immunobiology: The Immune System in Health and Disease, 4^{th} ed. Elsevier Science Ltd./Garland Publishing, (1999). Briefly, in a human antibody scaffold, the CDRs are embedded within a framework in the heavy and light chain variable region where they constitute the regions largely responsible for antigen binding and recognition. A human antibody variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra). Human light chains are classified as kappa and lambda light chains. Human heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the disclosure include all such classes or isotypes of human antibodies. The human light chain constant region can be, for example, a kappa- or lambda-type light chain constant region. The heavy chain constant region can be, for example, an alpha-, delta-, epsilon-, gamma-, or mu-type heavy chain constant regions. Accordingly, in exemplary embodiments, the antibody is an antibody of isotype IgA, IgD, IgE, IgG, or IgM, including any one of IgG1, IgG2, IgG3 or IgG4.

Antigen-binding proteins may have structures varying from that of a human antibody. In exemplary instances, the antigen-binding protein comprises only heavy chain fragments, e.g., heavy chain variable region, heavy chain constant region CH2, heavy chain constant region CH3. In various instances, the antigen-binding protein comprises a structure of a nanobody, such as those made by dromedary camel, llama, and shark. See, e.g., Leslie, Science, “Mini-antibodies discovered in sharks and camels could lead to drugs for cancer and other diseases”, 2018, at https://www.sciencemag.org/news/2018/05/mini-antibodies-discovered-sharks-and-camels-could-lead-drugs-cancer-and-other-diseases.

The following examples are given merely to illustrate the present invention and not in any way to limit its scope.

### EXAMPLES

### Example 1

This example describes an exemplary method of monitoring for the production of select antibodies in mice.

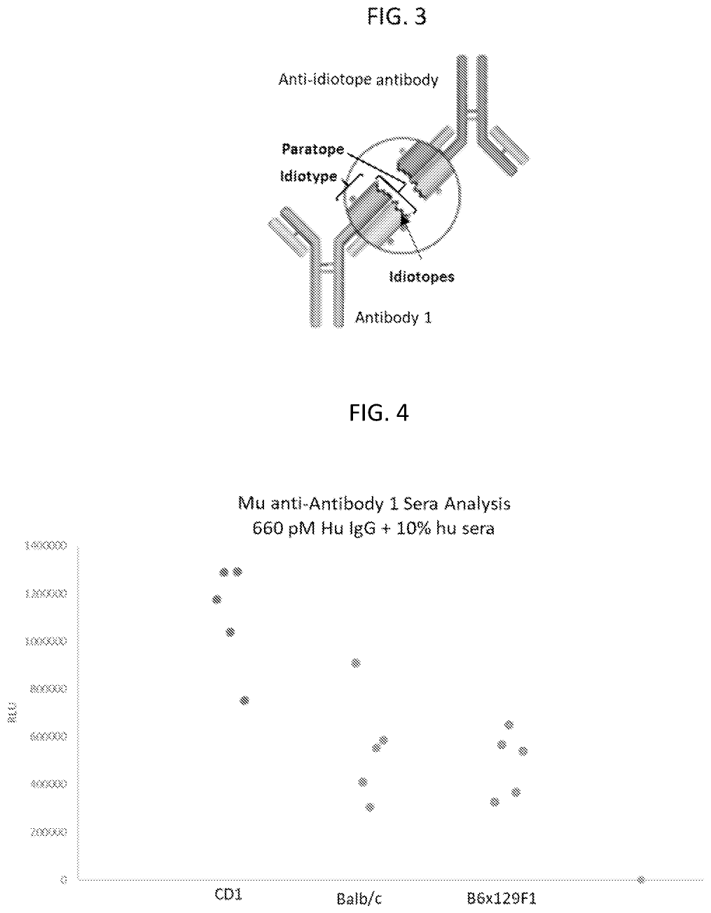

In this example, the select antibodies were anti-idiotypic antibodies (anti-ID abs) that bind to the idiotopes of a therapeutic human IgG antibody specific for PD-1 (hereinafter referred to as “Antibody 1”). Idiotopes are the unique structures formed by the variable regions of an antibody that are usually involved in binding to the antigen (the paratope). FIG. 3 illustrates the idiotopes and paratope of Antibody 1 as well as an anti-ID antibody.

Immunization Protocol

A soluble form of Antibody 1 was emulsified in adjuvant (Complete Freund's Adjuvant followed by the Sigma Adjuvant System® (SAS, Catalog No. S6322; Sigma-Aldrich, St. Louis, MO). The antibody-adjuvant mixture was then delivered into multiple strains of wild-type mice including Balb/c, CD1 and B6/129 mice. The complete immunization campaign consisted of three injections, delivered 2 weeks apart over the course of 38 days. The first immunization consisted of 50 μg of Antibody 1 emulsified in 100 μl Complete Freund's Adjuvant injected subcutaneously over 2 spots on the dorsal side of each mouse. Fourteen days later, 25 μg of Antibody 1 was suspended in 200 μl of Sigma Adjuvant System, and 100 μl of the mixture was injected in 2 spots subcutaneously on the dorsal side of each mouse and the remaining 100 μl was injected intraperitoneally. The 3^{rd} immunization was delivered 14 days later and was identical in route and adjuvant as the second, except that the total amount of Antibody 1 was reduced to 15 μg.

Bridging ELISA analysis of serum titers from each mouse was carried out as essentially described in Winters et al., mAbs 11(6): 1025-1035 (2019), to confirm antigen reactivity, and inform animal selection for final boost. As shown in FIG. 4, serum titer levels were highest among the CD1 mice, though all immunized mice, including the Balb/c, and B6/129 mice, exhibited serum titer levels greater than control (no sera). Four days prior to the non-terminal peripheral blood mononuclear cell (PBMC) harvest, 50 μg of Antibody 1 suspended in 150 μl of Phosphate Buffered Saline (PBS) was injected into each animal (N=12) via the intraperitoneal route to stimulate antigen-specific antibody secreting cells (ASCs).

Blood Collection and Enrichment

Blood from each animal was collected and processed for single-cell isolation and screening. Table 1 lists the mouse strain and blood volume harvested from each mouse.

TABLE 1

| | | Blood Volume |
|---|---|---|
| Mouse ID | Mouse Strain | Harvested* (μL) |
| 1 | CD1 | 180 |
| 2 | CD1 | 170 |
| 3 | CD1 | 200 |
| 4 | Balb/c | 120 |
| 5 | Balb/c | 170 |
| 6 | Balb/c | 160 |
| 7 | Balb/c | 140 |
| 8 | B6/129 | 180 |
| 9 | B6/129 | 100 |
| 10 | B6/129 | 150 |
| 11 | B6/129 | 150 |
| 12 | B6/129 | 210 |

*up to 7% of the complete blood volume

The collected blood was then processed to enrich for a B-cell pool. First, red blood cells (RBCs), platelets, and serum plasma were removed from the harvested blood using a RedSift Cell Processor instrument (Aviva Systems Biology, Corp., San Diego, CA). The EasySep™ Mouse B-cell Isolation Kit (STEMCELL Technologies, Inc., Vancouver, British Columbia) was then carried out following the manufacturer's procedure to further enrich for B-cells. An additional step using an in-house derived rat anti-murine IgM mAb (clone 8M3.1) was carried out to effectively remove naïve B cells expressing cell-surface IgM. This additional step allowed for further enrichment of antigen-specific, class-switched IgG secreting cells within the ASC population.

The enriched B-cell pool was incubated with fluorescently-labeled anti-CD138 antibodies to mark cells of the plasma B-cell lineage. High levels of CD138 expression has proven to be the most reliable indicator of IgG secretion from PBMC-derived B-cells, though it is contemplated herein that other cell surface markers (e.g., B220, CD19, IgG. TACI, SLAM7, BCMA, CD98, SCA-1, Ly6C1/2, etc.), or combinations of markers, could be used to identify specific B cell populations of interest. Tellier et al., Eur J Immunol 47(8): 1276-1279 (2017).

Single-Cell Screening Assay

The fluorescently-labeled cells were then loaded onto an OptoSelect™ chip (Berkeley Lights, Inc., Emeryville, CA) using the Beacon® Optofluidic Platform (Berkeley Lights, Inc., Emeryville, CA) which manipulates individual B cells into a separate pen of an OptoSelect™ chip through optoelectronic positioning (OEP). The chip had 3513 individual pens, each pen having a ˜740 picolitre capacity and a unique pen identification number. Chip-loaded ASCs were identified using the onboard optics of the Beacon® Optofluidic Platform through CD138 expression. Using this technique, individual B cells were sequestered into discrete pens of the chip such that antibodies secreted by an individual B-cell were isolated. The antibodies produced and secreted by one B-cell were not mixed with the antibodies produced and secreted by another B-cell. This “one ASC to one pen” relationship allowed for phenotypic characterization of the antibodies produced by a single B-cell, and since the ASC has a particular genotype, a phenotype to genotype association may be made. Due to the extremely small volume of the pens and the rapid secretion rate of plasmablasts and plasma cells (Wener Faver, et al., Eur J Immunol 23(8): 2038-2040 (1993), ASC-derived antibody concentrations within each pen increased quickly. Sufficient levels of antibody were reached within 5-15 minutes to allow screening for desired characteristics (e.g., phenotypes) of the select antibodies.

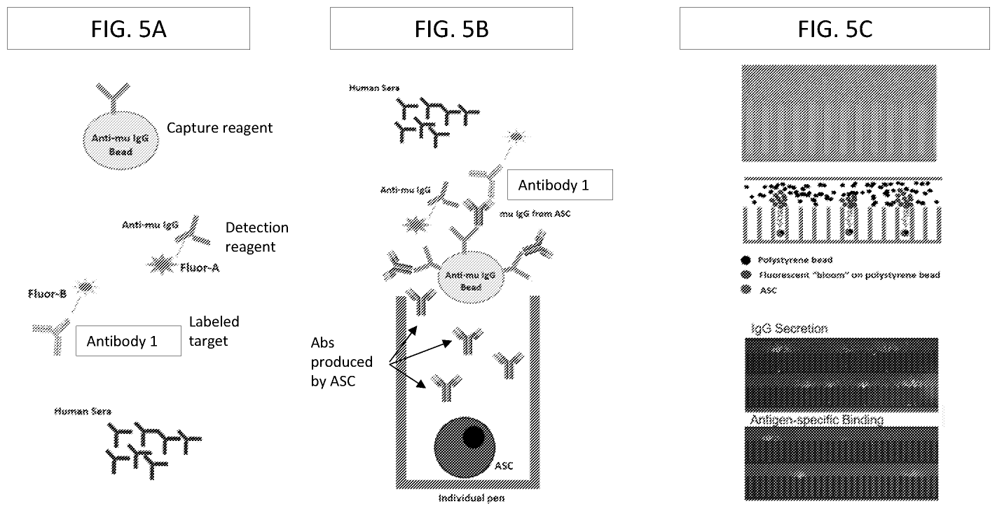

ASCs expressing relevant, antigen-specific antibodies (select antibodies) may be identified using a series of iterative homogenous screens. Depending on the desired antibody characteristics of the select antibodies, these screens may be simple binding assays (e.g. antigen binding, species cross reactivity, etc.) or designed to identify antibodies that meet additional design goals (e.g. ligand blocking, competition, function, etc.). Here, to identify ASCs secreting anti-ID abs directed against Antibody 1 (select antibodies), a homogenous, bead-based competition assay was performed. The assay is illustrated in FIGS. 5A-5C. In this assay, a capture reagent comprising an anti-mouse IgG antibody (anti-mu IgG) linked to a 3.2 μm polystyrene bead (Spherotech Inc, Lake Forest, IL) was mixed with a detection reagent comprising anti-mouse IgG labeled with Fluor A, a labeled target comprising Antibody 1 labeled with Fluor-B, and an excess of human sera (10% normal human sera). See FIG. 5A. The sera were included to provide competitive binding conditions. Without being bound to a particular theory, under such competitive binding conditions, antibodies specific to epitopes outside of the desired therapeutic IgG paratope do not bind to the labeled target (Fluor-B-labeled Antibody 1).

This assay mixture was then flowed into the chip microfluidic channel such that the beads were positioned at the mouth of each pen containing the individually sequestered ASCs (FIG. 5B). Pens containing cells that secreted antibodies were then detected using the fluorescent imaging capabilities of the Beacon® Optofluidic Platform (Berkeley Lights, Inc., Emeryville, CA) and a filter that permits detection of Fluor-A. As ASC-derived antibody levels increased, they diffused out of the mouth of the pen where they were captured (and concentrated) by the capture reagent. The increasing amounts of antibody bound to the beads, in turn, concentrated the anti-mouse IgG antibody conjugated to Fluor-A resulting in a characteristic fluorescent “bloom” pattern focused at the mouth of pens of interest (pens containing an ASC secreting IgG antibodies; FIG. 5C). Eighty-two pens harboring ASCs secreting IgG antibodies were identified by Fluor A blooms and their pen-identification numbers were recorded. To identify the ASCs secreting antibodies specific to Antibody 1 (select antibodies), a second fluorescent filter cube was used to detect Fluor-B signals. Twenty-three ASCs expressing select antibodies specific for Antibody 1 binding to the labeled target in the presence of the human sera were marked by Fluor B blooms (FIG. 5C).

Sequencing, Cloning, and Recombinant Expression

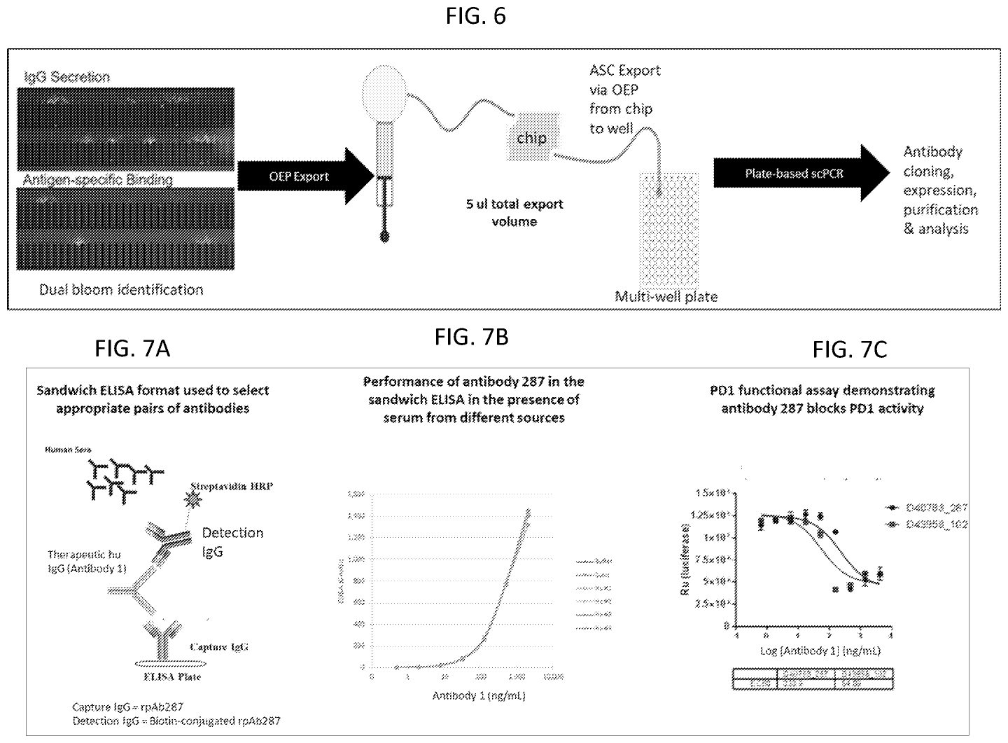

To validate the select antibodies, the 23 ASCs were individually moved out of the pens of the OptoSelect™ chip using OEP and exported into separate wells of a standard 96-well plate containing cell lysis buffer using the integrated microfluidics of the Beacon® Optofluidic Platform (FIG. 6). The sequences of the corresponding antibody heavy (HC) and light (LC) chain variable regions for the antibodies produced by the ASC of each well were determined via single-cell RT-PCR following the protocol as essentially described in Winters et al., 2019, supra. The sequences were then cloned into mammalian expression vectors carrying an antibody constant region. One vector carried the HC variable region and an antibody constant region and a second vector carried the LC variable region and an antibody constant region. The recombinant antibody HC/LC pairs were then transfected into 293T cells and expressed as soluble antibodies into the culture supernatant.

Antibodies in the culture supernatant were then tested for binding to Antibody 1 by Sandwich ELISA in the presence of human sera as essentially described in Winters et al., 2019, supra and as illustrated in FIG. 7A. Using this method, nine antibodies that possessed the desired characteristics and could act as anti-ID antibodies for Antibody 1 were identified. Of the 9 anti-ID candidates, a single antibody (Ab287) had the best profile displaying a potential lower limit of quantitation (LLOQ) of 0.5 ng/ml in clinical patient samples. The performance of Ab287 in the sandwich ELISA in the presence of serum from different sources is shown in FIG. 7B. As expected for an anti-ID that binds to the paratope of Antibody 1, the anti-ID antibody, Ab287, blocked Antibody 1 from binding to its target (PD-1) with an EC50 of 233.9 nM (FIG. 7C). Ab287 was expected to measure the free and bio-active Antibody 1 in clinical samples and therefore was selected for further development.

### Example 2

This example describes another exemplary method of monitoring for the production of select antibodies in mice.

Immunization Protocol

In this example, the select antibodies were anti-human EGFR antibodies. CD1 mice were immunized with the soluble extracellular domain of human EGFR (huEGFR) for a total of four immunizations spaced two weeks apart. The first immunization consisted of 50 sg of human huEGFR emulsified in 100 μl Complete Freund's Adjuvant injected subcutaneously over 2 spots on the dorsal side of each mouse. Fourteen days later, 25 sg of huEGFR was suspended in 200 μl of Sigma Adjuvant System, and 100 μl of the mixture was injected in 2 spots subcutaneously on the dorsal side of each mouse and the remaining 100 μl was injected intraperitoneally. The third immunization was delivered 14 days later and was identical in route and adjuvant as the second, except that the total amount of huEGFR was reduced to 15 sg. The fourth boost contained 50 sg of huEGFR in the absence of adjuvant and was delivered by both subcutaneous and intraperitoneal route.

Blood Collection and Enrichment

Blood was collected 1 to 8 days after the final boost. The ASCs were enriched using a magnetic CD138 positive selection kit (StemCell Technologies, Vancouver, Canada) following the manufacturer's procedure.

Single Cell Screening Assay

The enriched B-cells were mixed with a capture reagent comprising goat anti-human Fc linked to a bead, a detection reagent comprising the goat anti-human Fc antibodies labeled with Alexa 488 which produces a green fluorescent signal, and a labeled target comprising EGFR labeled with Alexa 594 which produces a red fluorescent signal. The mixture was then transferred to a single well of a 384-well plate and the components of the mixture were allowed to settle in the well for about 10 minutes.

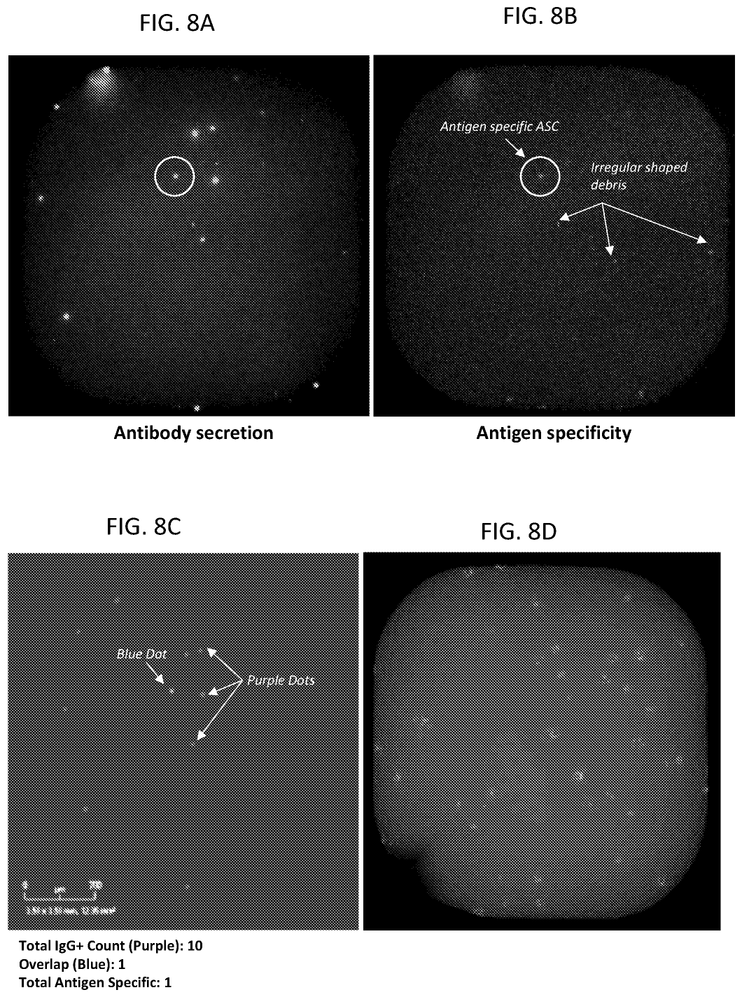

Cellular imaging was carried out to identify specific ASCs using the Incucyte Live-Cell Analysis System. FIG. 8A provides an exemplary image of the green fluorescent signal demonstrating antibody secretion and FIG. 8B provides an exemplary image of the red fluorescent signal demonstrating antigen specificity of antibodies. FIG. 8C provides an exemplary analyzed composite image of the same well depicted in FIGS. 8A-8B wherein the green fluorescent signal demonstrating antibody secretion is shown in magenta, the red fluorescent signal is shown in cyan and the overlap of green and red signals are shown in royal blue. From this imaging assay, 10 cells were found to demonstrate antibody secretion, while only 1 cell was demonstrated as secreting antibodies that were antigen (EGFR)-specific.

### Example 3

This example describes an alternative single-cell imaging assay to identify select ASCs, wherein the target is expressed by a cell in its native conformation.

Mice were immunized with CB-1 for the production of anti-CB-1 antibodies. Blood samples were collected from immunized mice and then enriched for IgG secreting B-cells as essentially described in Example 2. 293 T-cells transfected with a vector encoding full-length CB1 using 293fectin were washed with culture medium and then passed through a 40 μm strainer. A mixture of enriched B-cells, CB-1-expressing 293T cells and Goat anti-human Fc antibodies labeled with Alexa 488 were then added to the wells and allowed to settle as a monolayer. Specific ASCs were identified using the Incucyte imaging system to detect fluorescent signals at the surface of transfected cells. FIG. 8D provides an exemplary image of transfected cells labeled with multiple fluorescent spots at which antigen expressed by the 293T cell is bound to antibody produced by the B cell and labeled with the goat anti-human Fc antibody labeled with Alexa 488. These results demonstrated that the enriched B-cell pool contained cells secreting antibodies specific for CB-1.

### Example 4

This example describes an exemplary method of guiding antibody production in a non-human animal for the production of select antibodies. In this example, the select antibodies are human-cyno cross-reactive IgG antibodies specific for TNF-alpha.

To produce human-cyno cross-reactive antibodies, animals are immunized with alternating boosts of the human and cyno versions of the antigen. This immunization approach relies on the assumption that epitopes shared between the human and cyno antigens are consistently presented to the immune system during each boost, allowing persistent stimulation of relevant B-cells encoding cross-reactive antibodies. Reactivity to each antigen can be easily monitored using simple binding assays and the polyclonal serum from the immunized animals. However, since the animals have been immunized with both antigens, and the individual antigens harbor both common and unique epitopes, the polyclonal serum will contain antibodies reactive human antigen, antibodies reactive to cyno antigen and/or antibodies reactive to human and cyno antigens. Assaying the polyclonal serum does not allow for the determination that cross-reactive antibodies are present. Interrogation of isolated single ASCs derived from the PBMC population overcomes this problem. The single-cell assay screens for ASCs secreting truly cross-reactive antibodies.

Immunization Protocol

The complete immunization campaign consists of four injections, delivered 2 weeks apart over the course of 50 days. For the first and third injections, recombinant human TNF-alpha (Catalog No. 300-01A; PeproTech®; Rocky Hill, NJ) emulsified with Complete Freund's Adjuvant followed by Sigma Adjuvant System® (Catalog No. S6322; Sigma-Aldrich, St. Louis, MO) is used. For the second and fourth injections, recombinant cynomolgus monkey TNF-alpha (Catalog No. RP1021Y-005, Kingfisher Biotech, Inc., St. Paul, MN) emulsified with Complete Freund's Adjuvant followed by Sigma Adjuvant System® (Catalog No. S6322; Sigma-Aldrich, St. Louis, MO) is used. For the first injection, about 50 sg of human TNF is suspended in adjuvant and injected subcutaneously over 2 spots on the dorsal side of each mouse. Fourteen days later, a second injection using 50 sg of cynomolgus monkey TNF suspended in adjuvant was injected subcutaneously over 2 spots on the dorsal side of each mouse. Fourteen days after the second injection, a third injection comprising 25 sg of human TNF was suspended in 200 μl of Sigma Adjuvant System, and 100 μl of the mixture was injected in 2 spots subcutaneously on the dorsal side of each mouse and the remaining 100 μl was injected intraperitoneally. Fourteen days later, a fourth injection comprising 25 sg of cyno TNF was suspended in 200 μl of Sigma Adjuvant System, and 100 μl of the mixture was injected in 2 spots subcutaneously on the dorsal side of each mouse and the remaining 100 μl was injected intraperitoneally. Bridging ELISA analysis of serum titers from each mouse is carried out to confirm antigen reactivity and inform animal selection for non-terminal antibody discovery. Four days prior to the non-terminal peripheral blood mononuclear cell (PBMC) harvest, a solution comprising 25 sg of human TNF and 25 sg of cyno TNF suspended in 150 μl of PBS is injected into each animal (N=12) via the intraperitoneal route to stimulate antigen-specific antibody secreting cells (ASCs).

Blood Collection and Enrichment and Single Cell Screening Assay

Blood is collected from each mouse and enriched for B-cells as essentially described in Example 1. The labeled cells are loaded onto an OptoSelect™ chip (Berkeley Lights, Inc., Emeryville, CA) using the Beacon® Optofluidic Platform and individual B cells are sequestered into discrete pens of the chip so that antibodies secreted by an individual B-cell are isolated.

To identify ASCs secreting select antibodies (anti-TNF-antibodies reactive to human TNF and cyno TNF), the homogeneous, bead-based competition assay described in Example 1 is carried out. Here, a capture reagent comprising beads linked to an anti-mouse IgG are mixed with a detection reagent comprising Fluor-A labeled anti-mouse IgG, a labeled target comprising human TNF-labeled with Fluor-B, and an excess of human sera. This assay mixture is then flowed into the chip microfluidic channel such that the beads were positioned at the mouth of each pen containing the individually sequestered ASCs. Fluor-A blooms mark pens harboring ASCs secreting IgG antibodies, while Fluor B blooms mark pens harboring ASCs secreting antibodies which bind to human TNF. The pen ID numbers for pens marked by each bloom type are identified and recorded.

In a second part of the bead-based assay, a detection reagent comprising cyno TNF labeled with Fluor-C is added. Fluor-C blooms mark pens harboring ASCs secreting antibodies which bind to cyno TNF. The pen ID numbers of pends marked by Fluor C blooms are recorded.

Pens noted as positive for all three blooms (Fluor A bloom, Fluor B bloom, and Fluor C bloom) are selected as candidate ASCs secreting select antibodies. Candidate ASCs are individually moved out of the pens of the OptoSelect™ chip using OEP and exported into separate wells of a standard 96-well plate containing cell lysis buffer using the integrated microfluidics of the Beacon® Optofluidic Platform as essentially described in Example 1. HC and LC variable regions for the antibodies produced by each candidate ASC are determined via single-cell RT-PCR. The sequences are cloned into vectors and then the vectors are transfected into 293T cells. Antibodies in the culture supernatant is collected and then tested for cross-reactivity to human and cyno TNF in a functional assay.

If none of the pens are positive for all three blooms, pens that are double positive for Fluor A blooms and Fluor B blooms are identified. Alternatively, pens that are double positive for Fluor A blooms and Fluor C blooms are identified. The mice from which the blood containing the ASCs of the double positive pens are selected for a second immunization campaign. For those mice from which Fluor A and Fluor B double positive ASCs were obtained, the second immunization campaign comprises the same immunization campaign as the first campaign (described above) but the first and third injections are carried out with halved amounts of human TNF.

For those mice from which Fluor A and Fluor C double positive ASCs were obtained, the second immunization campaign comprises the same immunization campaign as the first campaign (described above) but the second and fourth injections are carried out with halved amounts of cyno TNF.

All steps following immunization (from blood collection to bead based assays) are subsequently carried out as described in this example. Pens noted as positive for all three blooms (Fluor A bloom, Fluor B bloom, and Fluor C bloom) are selected as candidate ASCs secreting antibodies with the target phenotype. The variable regions are sequenced, cloned into vectors, vectors are transfected into cells for recombinant antibody production and the recombinantly produced antibodies are tested for the target phenotype.

If triple positive pens are still not identified, a third immunization campaign is designed and carried out on the same mice receiving the second immunization campaign. In the third immunization campaign, for mice that are Fluor A/Fluor B double positive, the second and fourth injections are carried out with increased amounts of cyno TNF and the first and third injections are carried out with halved or quartered amounts of human TNF, and, for mice that are Fluor A/Fluor C double positive, the first and third injections are carried out with increased amounts of human TNF and the second and fourth injections are carried out with halved or quartered amounts of cyno TNF. Following the third campaign, all steps following immunization (from blood collection to bead based assays) are subsequently carried out as described in this example. Pens noted as positive for all three blooms (Fluor A bloom, Fluor B bloom, and Fluor C bloom) are selected as candidate ASCs secreting antibodies with the target phenotype. If triple positive pens are still not identified after the third campaign, a fourth immunization campaign is designed and carried out. The process is repeated until antibodies having the target phenotype are identified.

This method advantageously provides the capability of longitudinal in-life B-cell profiling to enable repertoire steering. The developing B-cell response of an immune animal is monitored in real-time and this information is used to iteratively modify the immunization strategy. Because this approach is non-terminal, it allows one to leverage the power of the immune system to continue to evolve the B-cell response towards a desired outcome without sacrificing the animal. Modifications to the immunization strategy include (but are not limited to) different forms of the immunogen, adjuvants, immunomodulatory agents, doses of the antigen, timing of the immunizations and routes of administration. In this scenario, the initial immunization attempts using the human antigen failed to elicit B-cells that produced antibodies that cross-reacted to the cyno antigen as determined by non-terminal ASC screening of PBMCs. Since this approach provides us with repertoire quality information, it can then be used to modify the immunization strategy. In this example, the immunogen could be switched from the human antigen to the cyno ortholog and the immunization campaign continued until B-cells expressing cross-reactive antibodies were identified. The animal that has elicited the desired B-cell repertoire could then be used for antibody generation using traditional strategies or non-terminal ASC methods as described here.

### Example 5

This example describes another exemplary method of guiding antibody production in a non-human animal for the production of select antibodies. In this example, the select antibodies are human-cyno cross-reactive IgG antibodies specific for Antigen X.

This example describes the identification of antibodies that cross-react to both human and cynomolgus (cyno) orthologs of Antigen X. The orthologs have low sequence homology and therefore the generation of cross-reactive antibodies is rare. Immunization with only one antigen may produce some cross-reactive antibodies but they would be below the level of detection of standard serum titers. Alternatively, co-immunization with both human and cyno antigens generates antibodies that predominantly bind to either the human or cyno ortholog, but few will cross-react. Standard serum titers do not discriminate between mice that have generated cross-reactive antibodies from those that have generated antibodies that independently bind either the human or cyno antigen. Single cell screening is therefore necessary to identify true cross-reactive antibodies in responding mice. This is coupled with selective amplification of the B cells of interest for efficient recovery.

Mice are immunized with the human version of Antigen X for a total of four injections spaced two weeks apart. For the first boost, 50 μgs of the human antigen X is emulsified in 100 μl of Freund's complete adjuvant and the mixture is administered subcutaneously. Fourteen days later, 25 sg of human Antigen X is suspended in 200 μl of Sigma Adjuvant System, and 100 μl is injected subcutaneously and 100 μl injected intraperitoneally. For the third injection, 15 sg of human Antigen X is emulsified in Sigma Adjuvant system and injected both subcutaneously and intraperitoneally, as described for the second boost. A final boost of 50 μgs of the human Antigen X is injected intraperitoneally in the absence of adjuvant.

Blood is collected for a final volume of 10% of the rodent body weight four days after the final boost. The CD138+ B cells are magnetically isolated and added to a mixture of capture reagent comprising anti-human IgG antibodies linked to beads, detection reagents comprising Alexa488-labeled anti-human IgG antibodies, and differentially labelled fluorescent human Antigen X and cyno Antigen X. The mixture is plated as a monolayer in microtiter plates and then incubated to allow antibody and antigen capture. Antigen specific cross-reactive ASCs are identified as dual staining fluorescent plaques using cellular imaging as essentially described in Example 2.

Animals that produced B cells that secrete cross-reactive antibodies are then immunized with alternating doses of the cyno and human antigens combined with Sigma Adjuvant System®, subcutaneously, once a week for an additional four weeks. Blood is collected three days after the last boost and the B cells isolated for screening against the human and cyno antigen.

Animals identified as having increased numbers of cross-reactive antibodies are euthanized for tissue harvest and antibody generation. Animals that have a low ratio of cross-reactive antibodies to mono-reactive are immunized with alternative doses of human and cyno orthologs of Antigen X for an additional 3 weeks followed by single cell screening for cross-reactive antibodies. This process is continued until a threshold % of ASCs producing select antibodies (human-cyno cross-reactive antibodies) is met.

The described approach may be applied to any antibody discovery campaign that requires cross-reactivity to multiple orthologs or paralogs of a protein. The need to generate antibodies that cross-react to different species is frequently required for efficacy and safety studies. Single B-cell screening could be applied to programs that require cross-reactivity to rat, rabbit, guinea pig, dog, cat or pigs as common examples.

### Example 6

This example describes a method of guiding antibody production in a non-human animal for the production of select antibodies. In this example, the select antibodies are antibodies that bind to only one paralog of a protein, Antigen X, but not to a closely related family member, Antigen Y.

Due to similarity between the Antigen X and Antigen Y, animals immunized with Antigen X show polyclonal serum cross-reactivity to both proteins. As such, direct single-cell screening is required to identify mice with the potential to generate a biased antibody response to the family member of interest. Selected animals are further immunized using an alternative immunization protocol to steer the immune response towards maximizing the generation of B cells producing antibodies reacting exclusively to Antigen X.

Rodents are immunized subcutaneously with Antigen X twice weekly for four weeks. The priming immunogen complex contains 10 sg of antigen combined with Freund's complete adjuvant while the boosting complex contains 5 sg of antigen combined with Sigma Adjuvant System®. Four days after the last boost, blood is collected, and CD138+ B cells separated from sera.

The cells are assayed through a single-cell assay as described in Example 1 and the cells are screened for binding to Antigen X using Fluor A-tagged Antigen X and/or screened for binding to Antigen Y using Fluor B-labeled Antigen Y. Fluor A blooms mark pens that contain an ASC secreting antibodies specific for Antigen X and Fluor B blooms mark pens containing an ASC secreting antibodies that bind to Antigen Y. Pens that are positive for Fluor A blooms only (and not positive for Fluor B blooms) are exported via OEP out of the pen and into a well for single cell PCR, as essentially described in Example 1. The antibodies produced by the ASCs would be assayed for binding to Antigen X and no binding to Antigen Y.

If pens are not single positive for Fluor A blooms, conserved domains of Antigen X are bioinformatically identified. Animals are immunized subcutaneously with the conserved Antigen X domains once weekly in combination with Sigma Adjuvant System® for an additional 3 boosts. The rodents are bled three days after the last boost and the B cells are enriched as essentially described in Example 1. Cells are screened at the single cell level to identify B cells that secrete antibodies that only bind to Antigen X and not to Antigen Y. In the single-cell assay, the conserved Antigen X domains labeled with Fluor C are used as the labeled target. Fluor C blooms mark the pens containing an ASC secreting select antibodies (antibodies specific for Antigen X that do not cross-react with Antigen Y).

Rodents with improved numbers of B cells expressing select antibodies are euthanized for tissue harvest. Mice that do not exhibit improved numbers are immunized a third time.

### Example 7

This example describes a method of guiding antibody production in a non-human animal for the production of select antibodies. In this example, the select antibodies are antibodies that out-compete native human PD-L1 and native human PD-L2 for binding to human PD-1.

Rodents are immunized with twice weekly with decreasing doses of PD-1 antigen as described in Example 6.

Individual B-cells are transferred into pens of a chip to achieve a one cell to one pen ratio. Bead-based assays are carried out using a capture reagent comprising beads linked to an anti-mouse IgG, a detection reagent comprising Fluor A-labeled anti-mouse IgG antibodies, a labeled target comprising human PD-1-labeled with Fluor-B, and an excess of human sera. This assay mixture is then flowed into the chip microfluidic channel such that the beads were positioned at the mouth of each pen containing the individually sequestered ASCs. Fluor-A blooms mark pens harboring ASCs secreting IgG antibodies, Fluor-B blooms mark pens harboring ASCs secreting antibodies which bind to PD-1. The pen ID numbers of pens marked by Fluor A blooms, Fluor B blooms, or double positive Fluor A and Fluor B blooms are recorded.

The bead-based assay is carried out a second time, only this time, increasing amounts of PD-L1 are added to the assay. Fluor A/Fluor B double blooms allow for the identification of pens containing an ASC which produces a PD-1-specific antibody and a maintained intensity of the signal in the presence of the PD-L1 indicates that the recombinantly produced antibodies out-compete PD-L1 for binding to PD-1. The pen number of the pen exhibiting a double bloom at a maintained intensity of the signal in the presence of PD-L1 is recorded.

The bead-based assay is carried out a third time, only this time increasing amounts of PD-L2 are added to the assay. Fluor A/Fluor B double blooms allow for the identification of pens containing an ASC which produces a PD-1-specific antibody and a maintained intensity of the signal in the presence of the PD-L2 indicates that the recombinantly produced antibodies out-compete PD-L2 for binding to PD-1. The pen number of the pen exhibiting a double bloom at a maintained intensity of the signal in the presence of PD-L2 is recorded. Desirably, there is a pen which is identified as containing an ASC which produces a PD-1-specific antibody which can outcompete both PD-L1 and PD-L2 for binding to PD-1.

Pens that are double positive for Fluor A and Fluor B blooms are noted and the variable regions of the HC and LC of the antibodies from these pens are sequenced. The sequences are cloned into vectors and the vectors are transfected into 293T cells for recombinant antibody production. Antibodies are collected from the supernatants of the 293T cell cultures and then tested for binding to recombinant PD-1 in the presence of increasing amounts of PD-L1. Here, the PD-1 is labeled with a fluorophore which emits a signal at a given wavelength, and the recombinantly produced antibodies are bound to beads as in an immunoprecipitation assay. Labeled PD-1 is mixed with the antibodies-bound to the beads. The beads are washed for non-specific binding. The immune complexes comprising labeled PD-1 and recombinantly produced antibodies are detected upon detection of the signal at the given wavelength. This procedure is then carried out with increasing amounts of PD-L1 and/or PD-L2. A maintained intensity of the signal in the presence of the PD-L1 and/or PD-L2 indicates that the recombinantly produced antibodies out-compete PD-L1 and PD-L2 for binding to PD-1.

Animals that generate B cells that bind to PD1 but do not fully compete with PD-L1 or PD-L2 are immunized with 2.5 μg of PD1 in combination with Sigma Adjuvant System® for 3 additional boosts. The mice are bled three days after the last boost and the isolated B cells are screened as described above.

### Example 8

This example describes a method of guiding antibody production in a non-human animal for the production of select antibodies. In this example, the select antibodies are antibodies with a particular binding affinity for the Antigen X.

The goal of this experiment is to identify an antibody that binds to Antigen X with sub-picomolar affinity. Affinity can only be measured on a clonal source of antibody and therefore the sera cannot be used to identify mice that have generated high affinity antibodies. Traditionally, mice are euthanized to obtain the B cells for hybridoma fusion and characterization, precluding any additional immune steering. Combining real-time, non-terminal B cell sampling and interrogation with an adaptive immunization strategy provides a significant advantage over traditional methods for generating high affinity antibodies as it leverages the competitive in vivo environment to force and then guide the evolution of higher affinity B cell clones.

Rodents are immunized subcutaneously with decreasing doses of Antigen X, every two weeks, for a total of four boosts. The priming immunogen contains 40 μg of Antigen X combined with Freund's complete adjuvant. The subsequent three boosts contain either 20 μg, 10 μg or 5 μg of Antigen X in combination with Sigma Adjuvant Systems®. Four days after the final boost, blood is harvested from the rodents and the collected blood samples are enriched for B-cells as essentially described in Example 1. The cells are penned using a microfluidic device and screened for binding to fluorescently labeled Antigen X, as essentially described in Example 1. ASCs producing anti-Antigen X antibodies are identified and subsequently exported out of pens and into wells for molecular recovery and the affinity of the recombinant clones are determined as essentially described in Example 1.

Affinity is determined by either KinExA (Sapidyne) or the Carterra high throughput screening device (Carterra).

Animals that generated B cells expressing high affinity antibodies are then boosted once a week with 2.5 μg of antigen in combination with Sigma Adjuvant System® for 3 additional boosts. The mice are bled three days after the last boost and the isolated B cells are screened for binding to Antigen X as essentially described in Example 1.

The B cells are then be exported for another round of sequencing, cloning, expression and affinity characterization. Rodents with B cells that meet the affinity bar will be euthanized for tissue harvest. Rodents that generated B cells that fall short of the affinity requirement will be boosted with 2.5 μg of antigen in combination with Sigma Adjuvant System®, once a week, for 3 additional boosts. The animals are screened and boosted until the design goal is met. In one round of screening, the enriched B-cells are subjected to the single-cell assay described in Example 2, except that the labeled target comprises Antigen X labeled with Alexa 594, instead of EGFR. Spots that demonstrate overlapping signals identify the ASC secreting antibodies specific for Antigen X. The single cell imaging assay is repeated with an amount of Antigen X labeled with Alexa 488 that is about 10-fold less than the amount of Antigen X labeled with Alexa 594. Those spots that retain the overlapping signal (from Alexa594 and Alexa 488) demonstrate higher affinity for Antigen X and thus display the desired high affinity for the target.

### Example 9

This example describes a method of guiding antibody production in a non-human animal for the production of select antibodies. In this example, the select antibodies are domain specific antibodies.

This example describes the identification of antibodies to Domain 1 of a multidomain human protein, Protein Z. Immunization with the full extracellular domain of Protein Z generates an unbalanced immune response with some domains being highly overrepresented and Domain 1 antibodies undetectable at the polyclonal serum titer level. Immunization with Domain 1 alone does not generate antibodies that can recognize the natural extracellular domain. Single cell screening is required to identify mice that have generated the rare Domain 1 antibodies after immunization with the natural extracellular domain of the protein. These B cells are amplified with subsequent boosting of a Domain 1 peptide and full-length protein in order to expand the Domain 1 reactive B cells but not generate a de novo response to the peptide alone.

Mice are immunized with the Protein Z for a total of four boosts spaced two weeks apart. The priming boost contains 50 sg of the antigen emulsified in Complete Freund's Adjuvant (CFA) and is administered subcutaneously. The subsequent boosts are emulsified in SAS and administered half intraperitoneally and half subcutaneously with 25 and 15 sg of antigen respectively. The mice are then boosted with 50 sg of the antigen in PBS and blood is collected after 4 days. The CD138+B cells are isolated and added to a mixture of IgG capture beads and differentially labelled fluorescent Domain 1 peptide and full extracellular domain. The mixture is plated as a monolayer in microtiter plates and then incubated to allow antibody and antigen capture, as essentially described in Example 2. ASCs producing Domain 1-specific antibodies are identified as dual staining fluorescent plaques using cellular imaging (Example 2).

Mice that have generated Domain 1-specific B cells are boosted with a 5 sg of a Domain 1 peptide twice weekly for two weeks. The mice are then boosted with 50 sg of the full soluble extracellular domain in PBS and blood collected after 4 days. The mice are screened for both binding to Domain 1 as well as full length protein. Animals of interest are euthanized for tissue processing and screening. The process is repeated for other mice until the design goal is met.

The described approach could be applied to any antibody discovery campaign where the immune response is predominantly directed against a region of the protein that is of low interest. A common example is a protein with an immunodominant region that is overrepresented in the antibody repertoire. The immune response will need to be steered away from that domain and onto the region of interest.

### Example 10

This example describes a method of guiding antibody production in a non-human animal for the production of select antibodies. In this example, the select antibodies are antibodies that bind to a multimerization domain of a multimeric antigen.

This experiment describes the identification of antibodies that bind to a heterotrimeric transmembrane protein. Immunization with the native protein fails to elicit an immune response. Mice immunized with the soluble domain alone do generate an immune response, but the antibodies do not recognize the native conformation of the protein. The mice are immunized with a series of immunogens made with increasingly native-like structure: immunogen 1 contains the proteins composed of the extracellular domain of antigen X; immunogen 2 contains proteins linked to a multimerization domain to form a heterotrimeric complex and immunogen 3 contains DNA encoding the full complex.

Rodents are immunized subcutaneously with 5 sg of immunogen 1 in complex with Sigma Adjuvant System®. The animals are boosted twice weekly for a total of 6 boosts. Blood is collected from the mice four days after the last boost and the CD138+ B cells are magnetically isolated. The cells are added to a mixture of IgG capture beads and differentially labelled fluorescent immunogen 1 and immunogen 2. The mixture is plated as a monolayer in microtiter plates and then incubated to allow antibody and antigen capture as essentially described in Example 2. Antigen specific ASCs are identified as dual staining fluorescent plaques using cellular imaging (Example 2). The mice that generate the rare antibodies that naturally cross-react to immunogen 2 are identified for further steering. These mice receive an additional twice weekly boosts with 5 sg of immunogen 2 in complex with Sigma Adjuvant System® for a total of 6 boosts to amplify the response. The rodents are bled four days after the last boost and the B cells are isolated for single cell screening against immunogen 2 and immunogen 3. Animals that carry B cells that encode antibodies that recognize immunogen 3 are genetically immunized with plasmids encoding the full complex. These rodents are boosted twice weekly with gene gun bullets for a total of 6 boosts. Blood is collected from the mouse for single cell screening and mice of interest are euthanized and the tissue harvested. Mice generating a weaker response are boosted and screened until the design goal is met.

### Example 11

This example describes an exemplary application of the single-cell assay for ranking ASCs by affinity of the antibodies secreted by the ASCs.

Hybridoma clones producing EGFR-specific antibodies were isolated and the EGFR binding characteristics for each clone were determined on the Octet® Bio-Layer Interfermetry platform (Satorius). Five hybridoma clones producing antibodies ranging in EGFR binding affinities (KD 4.7×10⁻¹⁰ to 1.1×10⁸) were selected for evaluation in the single cell assay. The selected hybridoma clones and the binding characteristics of the antibodies produced by each are listed in Table 2.

TABLE 2

| Hybridoma name | KD (M) | kₒₙ(1/ms) | K_{dissociation}(1/s) |
|---|---|---|---|
| 7.35.4 | 4.7 × 10⁻¹⁰ | 3.9 × 10⁵ | 1.8 × 10⁻⁴ |
| 12B4.1 | 2.4 × 10⁻⁹ | 7.5 × 10⁵ | 1.8 × 10⁻³ |
| 1C2.1 | 9.5 × 10⁻⁹ | 5.3 × 10⁵ | 5.0 × 10⁻³ |
| 7C11.1 | 1.1 × 10⁻⁸ | 5.3 × 10⁵ | 5.7 × 10⁻³ |
| 2G8.1 | 2.3 × 10⁻⁸ | 7.5 × 10⁵ | 1.8 × 10⁻² |

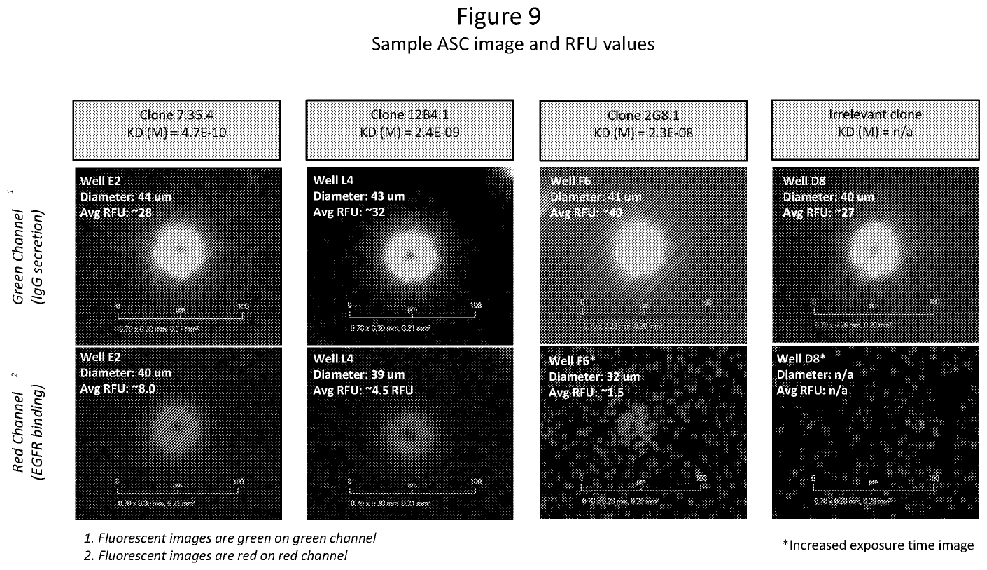

Single cell screening assays were carried out with the hybridoma clones of Table 2 as described in Example 2. Briefly, clones of hybridoma 12B4.1 were mixed with a capture reagent comprising goat anti-human Fc linked to a bead, a detection reagent comprising the goat anti-human Fc antibodies labeled with Alexa 488, which produces a green fluorescent signal, and a labeled target comprising EGFR labeled with Alexa 594, which produces a red fluorescent signal. The mixture comprising capture reagent, detection reagent, labeled target and 12B4.1 clones was then transferred to a single well of a 384-well plate. These steps were carried out with clones of each of the hybridoma clones of Table 2 so that each well of the plate contained a mixture comprising clones of a single hybridoma (e.g., one well for Hybridoma 1C2.1 clones, one well for Hybridoma 7C11.1 clones, one well for Hybridoma 2G8.1 clones, one well for Hybridoma 12B4.1 clones and one well for Hybridoma 7.35.4 clones). After the components of the mixture settled into the well, cellular imaging was carried out using the Incucyte Live-Cell Analysis System, and relative fluorescence unit (RFU) values for the green fluorescence and red fluorescence were determined for six individual cells (ASCs) of each well. Example images are shown in FIG. 9. The RFU values were recorded and the ratio of green RFU (representing IgG secretion) to red RFU (representing EGFR binding) were determined for normalization of the data (Table 3). RFU normalization is important, as IgG secretion levels can be influenced by cell health, cell cycle and other properties of the ASC. The RFU ratios for the six clones of a single hybridoma were averaged and recorded as Ratio Avg (Table 3).

TABLE 3

| | | Avg IgG | Avg EGFR | | | |
|---|---|---|---|---|---|---|
| Hybridoma | | RFU (Green | RFU (Red | Green/Red | | |
| Name | ASC # | Channel) | Channel) | RFU | % CV | Ratio Avg |
| 7.35.4 | 1 | 15.8 | 3.5 | 4.5 | 0.3 | 4.0 |
| | 2 | 28.7 | 6.4 | 4.5 | | |
| | 3 | 29.0 | 8.0 | 3.6 | | |
| | 4 | 12.2 | 3.2 | 3.8 | | |
| | 5 | 43.7 | 11.5 | 3.8 | | |
| | 6 | 30.7 | 7.8 | 3.9 | | |
| 12B4.1 | 1 | 16.8 | 2.7 | 6.3 | 0.6 | 6.0 |
| | 2 | 14.4 | 2.6 | 5.6 | | |
| | 3 | 28.2 | 4.0 | 7.0 | | |
| | 4 | 11.9 | 2.3 | 5.3 | | |
| | 5 | 15.5 | 2.5 | 6.3 | | |
| | 6 | 11.6 | 2.1 | 5.5 | | |
| 1C2.1 | 1 | 22.4 | 2.1 | 10.7 | 1.6 | 12.4 |
| | 2 | 42.0 | 2.8 | 15.2 | | |
| | 3 | 25.2 | 2.2 | 11.7 | | |
| | 4 | 22.0 | 2.0 | 11.2 | | |
| | 5 | 28.4 | 2.0 | 13.9 | | |
| | 6 | 25.2 | 2.1 | 11.8 | | |
| 7C11.1 | 1 | 18.1 | 1.8 | 10.1 | 2.0 | 13.9 |
| | 2 | 25.5 | 1.7 | 15.0 | | |
| | 3 | 34.6 | 2.2 | 15.7 | | |
| | 4 | 22.4 | 1.8 | 12.3 | | |
| | 5 | 31.1 | 2.1 | 15.0 | | |
| | 6 | 38.2 | 2.5 | 15.4 | | |
| 2G8.1 | 1 | 37.2 | 1.9 | 19.6 | 2.6 | 18.5 |
| | 2 | 32.0 | 2.0 | 16.3 | | |
| | 3 | 24.5 | 1.7 | 14.8 | | |
| | 4 | 36.1 | 2.1 | 17.6 | | |
| | 5 | 44.3 | 2.0 | 22.6 | | |
| | 6 | 39.0 | 1.9 | 20.3 | | |
| Irrelevant | 1 | 23.3 | 1.1 | 21.2 | 4.8 | 22.2 |
| Clone | 2 | 20.4 | 1.2 | 17.3 | | |
| | 3 | 26.9 | 1.0 | 26.9 | | |
| | 4 | 18.4 | 1.2 | 14.8 | | |
| | 5 | 34.0 | 1.3 | 26.2 | | |
| | 6 | 32.9 | 1.2 | 27.0 | | |

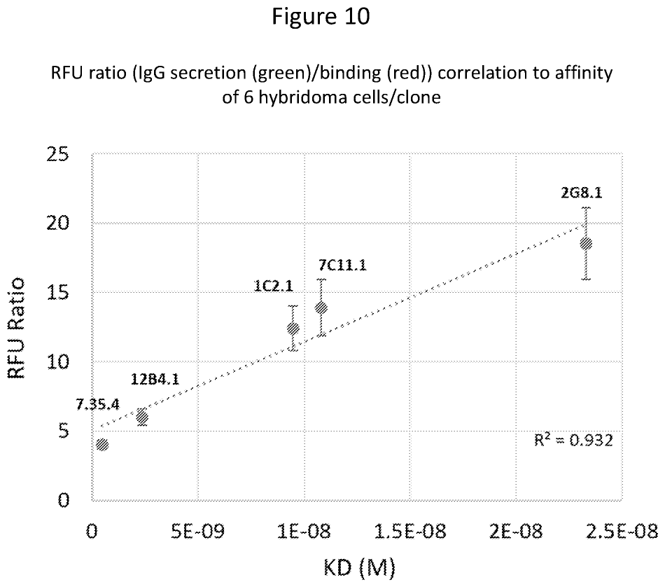

The Ratio Avg for each hybridoma was then plotted as a function of its KD value from Table 2 (FIG. 10). As shown in FIG. 10, the Ratio Avg and the KD values (as determined on the Octet® Bio-Layer Interferometry platform (Satorius)) correlated with statistical significance (R²=0.932). Taken together, these results demonstrate that the normalized RFU of a single clone is useful in ranking antibody affinity. These results further suggest that the presently disclosed single cell assay may be used to rank individual ASCs according to the affinity of their secreted antibody.

### Example 12

This example describes a method of guiding the immune response in mice to generate antibodies that cross-react to both human and cynomolgus monkey (cyno) orthologs of an antigen. This example explores the impact of different immunization strategies on the formation of cross-reactive antibodies and the ability of the single cell screening strategy to detect those changes. This example also demonstrates the application of the single cell assay in immune steering.

Immunization

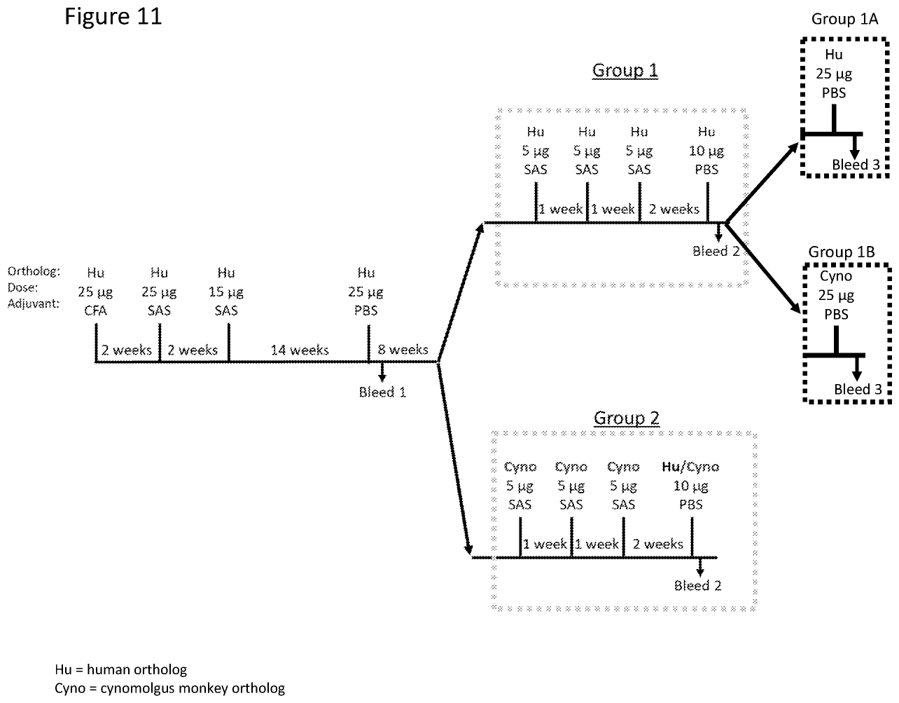

CD1 mice were immunized every 2 weeks with the human ortholog of the antigen. For the initial boost, mice were immunized subcutaneously with 25 sg of human antigen in complete Freund's adjuvant (CFA). The second boost contained 25 sg of human antigen combined with 50% Sigma Adjuvant System (SAS) and was administered half subcutaneously and half intraperitoneally. The third dose had 15 sg of human antigen combined with 50% SAS and was administered half subcutaneously and half intraperitoneally. The mice were rested for 14 weeks and then boosted with 25 μg of human antigen (in phosphate buffered saline (PBS)) without adjuvant into the peritoneal cavity. Blood was collected four days after the boost for serology and single cell screening (FIG. 11, Bleed 1).

The mice were then divided into two groups: Group 2 was immune steered toward production of human/cyno cross-reactive antibodies by boosting with cyno antigen subcutaneously once a week, while Group 1 served as a control group and was boosted with human antigen. The mice of both groups were bled four days after the eighth boost (FIG. 11, Bleed 2) and the blood was prepared for analysis.

Following the eighth boost, mice from Group 1 were divided into two subgroups (Groups 1A and 1B) and given a single unadjuvanted boost of either cyno antigen (Group 1B) or human (Group 1A) antigen. The mice of both subgroups were bled four days after the boost (FIG. 11, Bleed 3) and the blood prepared for analysis.

Blood Preparation and Cell Enrichment

Blood was collected at the indicated times in FIG. 11 (Bleed 1, Bleed 2 and Bleed 3). At each instance, blood was centrifuged to separate sera from the blood cells. Sera was used in the Serum Titer Analysis described below and the blood cells were enriched for ASCs by enriching for CD138+ B-cells as using a modified version of the standard protocol of a CD138 enrichment kit ((STEMCELL Technologies, Inc., Vancouver, British Columbia)).

Single Cell Screening Assay

Single cell screening assays were carried out with an enriched CD138+ B-cell population as described in Example 2. Briefly, the enriched CD138+ B-cell population was mixed with a capture reagent comprising a goat anti-mouse IgG Fc linked to a 3.4 μm polystyrene bead (Spherotech Inc, Lake Forest, IL), cyno antigen labeled with Alexa 488, which produces a green fluorescent signal, human antigen labeled with Alexa 594, which produces a red fluorescent signal, and brought to final concentrations using B-cell media as diluent. The use of the different fluorescent signals (green for cyno antigen and red for human antigen) allowed the single cell assay to distinguish between single cells binding to only the human ortholog, single cells binding to only the cyno ortholog, and single cells binding to both orthologs. The mixture was transferred to single wells of a 384-well plate, and the final enriched B-cell concentration was around 2-3 μL of cell mixture per well. After the components of the mixture were allowed to settle in the well for about 10 minutes, cellular imaging was carried out using the Incucyte Live-Cell Analysis System. RFU values for the green fluorescence and red fluorescence were determined.

Serum Titer Analysis

Serum from Bleeds 1 and 3 was diluted to final concentrations of 1:100, 1:1000 and 1:10,000 and then added to beads with captured biotinylated antigen that were plated in a V-bottom 96-well plate. The mixture was incubated for 1 hour at room temperature. The beads were then washed and resuspended in 30 sg of goat anti-mouse IgG Fc (Jackson Immunoresearch) at a final concentration of 5 sg/mL. After a 15-minute incubation, the beads were washed with and resuspended in FACS buffer. The plate was then prepared for flow cytometry.

Results and Discussion

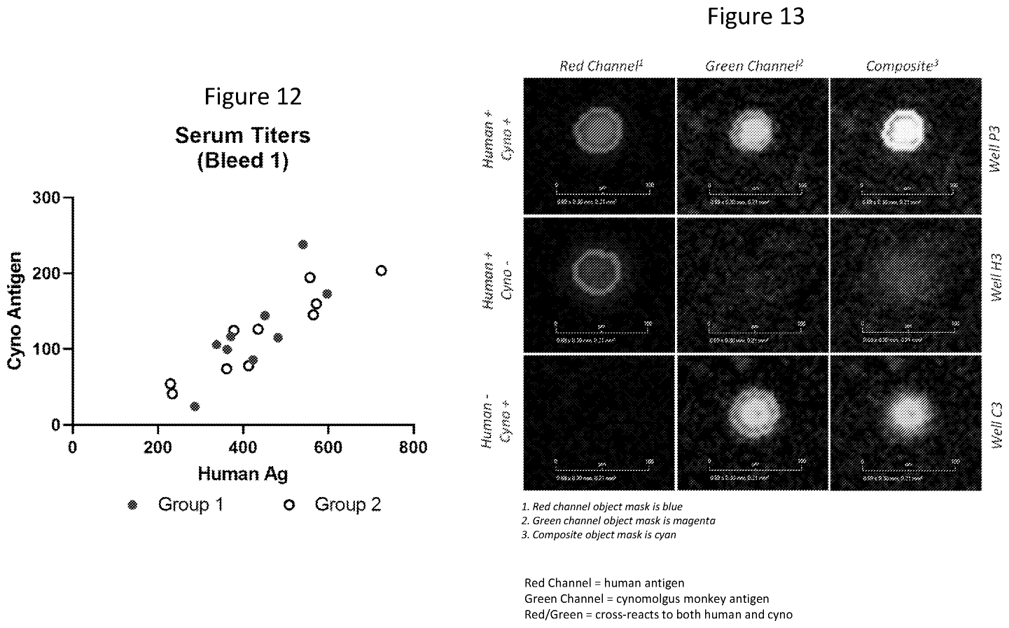

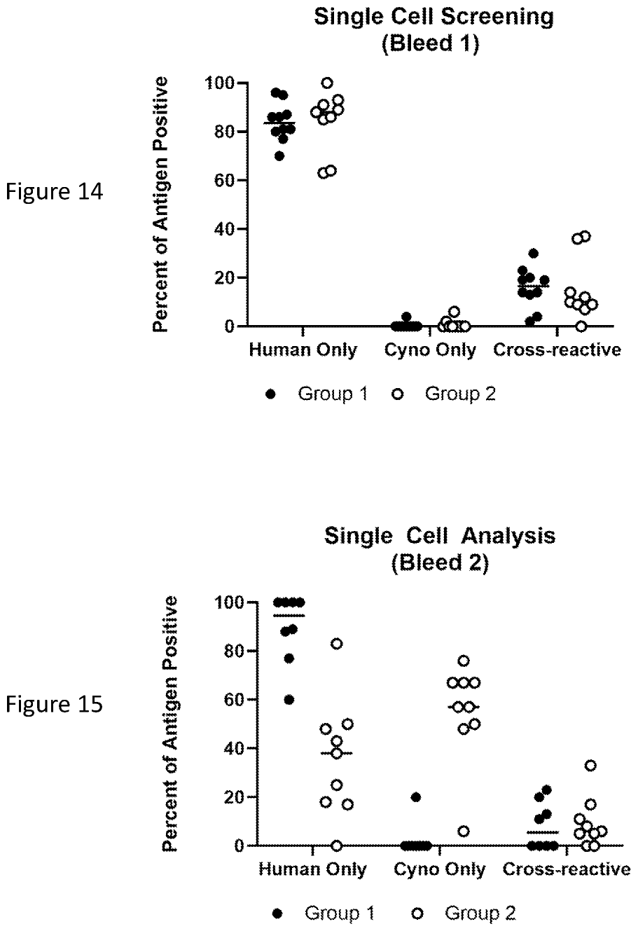

Groups 1 and 2 mice were initially boosted in identical manner, and serum titer analysis indicated that all mice generated a robust immune response to both human and cyno orthologs but the titer data could not distinguish cyno only from human-cyno cross reactive binders (FIG. 12, Bleed 1). The single cell screen was used to identify the percentage of antibodies that bound to human only, cyno only or both cyno-human orthologs in each individual mouse (FIGS. 13 and 14, Bleed 1). While the dominant immune response was to the human ortholog, cross-reactive antibodies could be readily identified in some mice (FIG. 14).

The immunization conditions were then shifted to determine if the single cell screen could detect changes in the antibody repertoire after immune steering. Group 1 mice continued to receive boosts with the human ortholog while Group 2 mice were boosted with the cyno ortholog (FIG. 11, Bleed 2). The single cell screening assay was able to detect a strong shift in the percentage of antibodies that bound to the cyno vs human ortholog in Group 2 mice (FIG. 15) with cyno only antibodies now dominating the immune response. Group 1 control mice had a similar response at Bleeds 1 and 2 (FIG. 14 vs 15). Neither group had a significant change in the generation of cross-reactive antibodies. These data demonstrate that the single cell screening assays have the resolution to detect changes in the immune response and that small changes in the immunization strategy could shape the antibody repertoire.

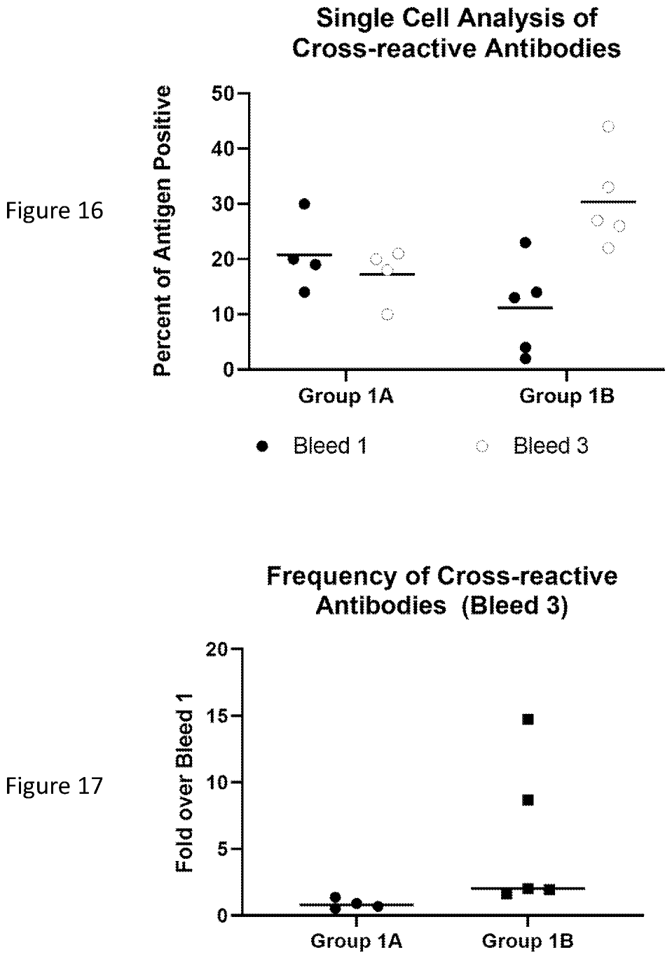

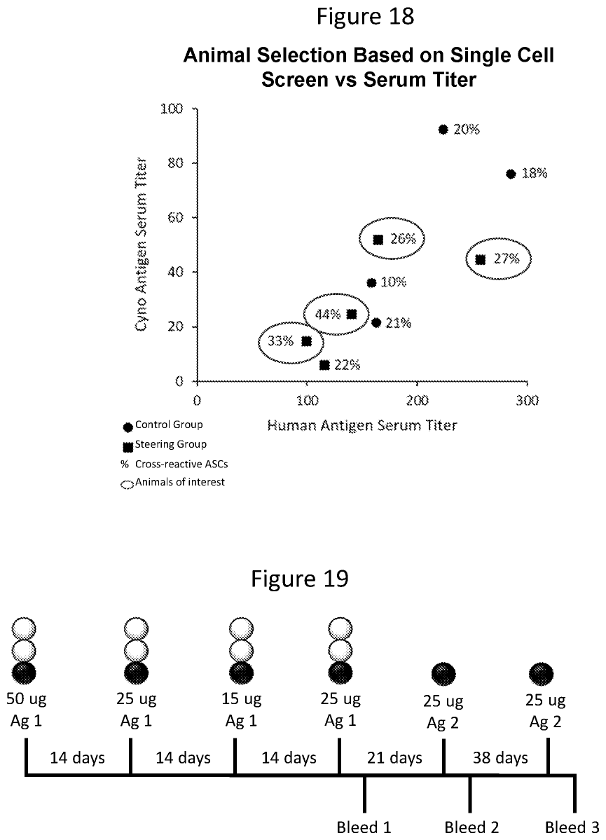

Group 1 mice were then boosted one final time to determine if the immune response could be steered toward an increase in cross-reactive antibodies with a minimal cyno only response. The previous data demonstrated that there would be a strong de novo response to the cyno antigen and multiple boosts would not generate the required response. Therefore, Group 1 mice received a single boost with 25 sg of protein and no adjuvant to minimize a de novo response. Group 1A received a boost with the human ortholog and Group 1B with the cyno ortholog. Mice that received the human boost (Group 1A) had no change in the percentage of cross-reactive antibodies relative to Bleed 1 (FIG. 16-17). The lack of variation in the mice that were only boosted with human antigen highlights the reproducibility of the single cell screen. By contrast, the cyno boosted group (Group 1B) had a significant increase in the percentage of cross-reactive antibodies (FIG. 16) with a range of 2-16-fold increase in cross-reactive antibodies (FIG. 17). There was a minimal percentage of antibodies that bound to cyno alone indicating that the single boost minimized de novo production of cyno only antibodies. All mice in the cyno boosted group (Group 1B) now met design goals with the top animals not discernable using standard polyclonal serology (FIG. 18). This is likely due to the serum containing a polyclonal mixture of all antibodies generated throughout the immunization campaign with the newly formed ASCs only contributing a very small percentage of the repertoire.

Taken together, these results suggest that different immune responses can be achieved by immune steering, by, e.g., boosting with a different ortholog antigen. These results further support that, unlike polyclonal serum titer analyses which cannot differentiate between antibodies that bind to human ortholog only vs antibodies that bind to cyno ortholog only vs antibodies that bind to both orthologs (cross-react to both human and cyno orthologs), the single cell screening assays of the present disclosure have the needed resolution and sensitivity to detect changes in the immune response and immune repertoire which allow tracking or monitoring of antibody production, even when only small changes in the immunization strategy occur.

### Example 13

This example describes a method of steering the immune response to increase the proportion of antibodies that cross-react to both human and cyno orthologs of a multi-domain protein.

Previous attempts to produce human/cyno cross-reactive antibodies to an antigen that is a multi-domain protein were made. The cyno and human orthologs have less than 80% homology. In the previous attempts, boosts of human antigen alternated with boosts of cyno antigen, which approach led to a strong de novo response to each ortholog protein but very few antibodies were cross-reactive. Here, in this example, mice were initially boosted with the full extracellular domain of the human protein (Antigen 1) and subsequently boosted with a subdomain of the protein (Antigen 2). The cyno subdomain and the human subdomain have greater than 80% homology.

Immunizations

CD-1 mice were immunized with Antigen 1 every two weeks for a total of four boosts (FIG. 19). The first boost was 50 sg of Antigen 1 emulsified in CFA and was injected subcutaneously. The second boost was 25 sg of Antigen 1 combined with 50% SAS, half delivered subcutaneously and the other half delivered intraperitoneally. The third boost was 15 sg of Antigen 1 combined with 50% SAS and was injected half subcutaneously and half intraperitoneally into the mice. The fourth boost was 25 sg of Antigen 1 in the absence of adjuvant. Blood was collected four days later for single cell screening (Bleed 1). Two additional boosts were administered following Bleed 1. Each was with 25 sg of Antigen 2 and four days after each, blood was collected (Bleed 2 and Bleed 3).

Blood Preparation and Cell Enrichment

Blood was collected three times throughout the immunization campaign as indicted in FIG. 19 (Bleed 1, Bleed 2, Bleed 3). At each instance, blood was centrifuged to separate sera from the blood cells. Sera was used in the Serum Titer Analysis described below and the blood cells were enriched for ASCs by enriching for CD138+ B-cells as using a modified version of the standard protocol of a CD138 enrichment kit ((STEMCELL Technologies, Inc., Vancouver, British Columbia)).

Single Cell Screening Assay

Single cell screening assays were carried out with an enriched CD138+ B-cell population as described in Example 2. Briefly, the enriched CD138+ B-cell population was mixed with a capture reagent comprising a goat anti-mouse IgG Fc linked to a 3.4 μm polystyrene bead (Spherotech Inc, Lake Forest, IL), cyno antigen labeled with Alexa 488 which produces a green fluorescent signal, human antigen labeled with Alexa 594 which produces a red fluorescent signal, with a HexaHis protein at least 100-fold molar excess to compete out His tag specific titers (GenScript RP11737), and brought to final concentrations using B-cell media as diluent. The use of the different fluorescent signals (green for cyno antigen and red for human antigen) allowed the single cell assay to distinguish between single cells binding to only the human ortholog, single cells binding to only the cyno ortholog, and single cells binding to both orthologs. The mixture was transferred to single wells of a 384-well plate, and the final enriched B-cell concentration was around 2-3 μL of cell mixture per well. After the components of the mixture were allowed to settle in the well for about 10 minutes, cellular imaging was carried out using the Incucyte Live-Cell Analysis System. RFU values for the green fluorescence and red fluorescence were determined.

Serum Titer Analysis

Serum was diluted to final concentrations of 1:100, 1:1000 and 1:10,000 and then added to beads with captured biotinylated antigen that were plated in a V-bottom 96-well plate. The mixture was incubated for 1 hour at room temperature. The beads were then washed and resuspended in 30 μg of goat anti-mouse IgG Fc (Jackson Immunoresearch) at a final concentration of 5 μg/mL. After a 15-minute incubation, the beads were washed with and resuspended in FACS buffer. The plate was then prepared for flow cytometry.

Results and Discussion

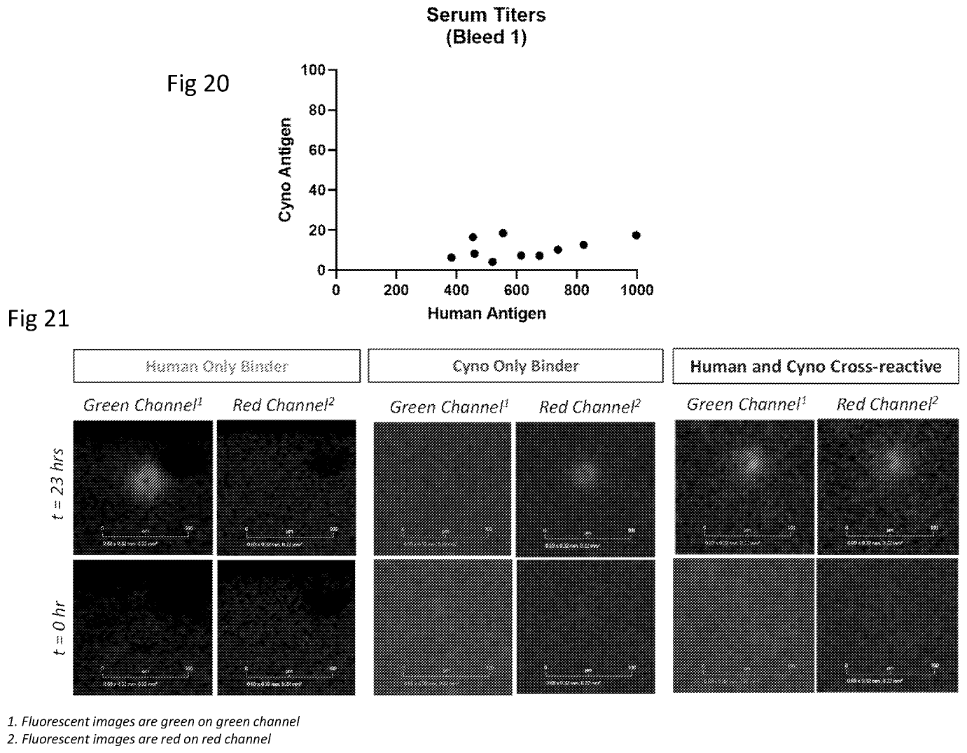

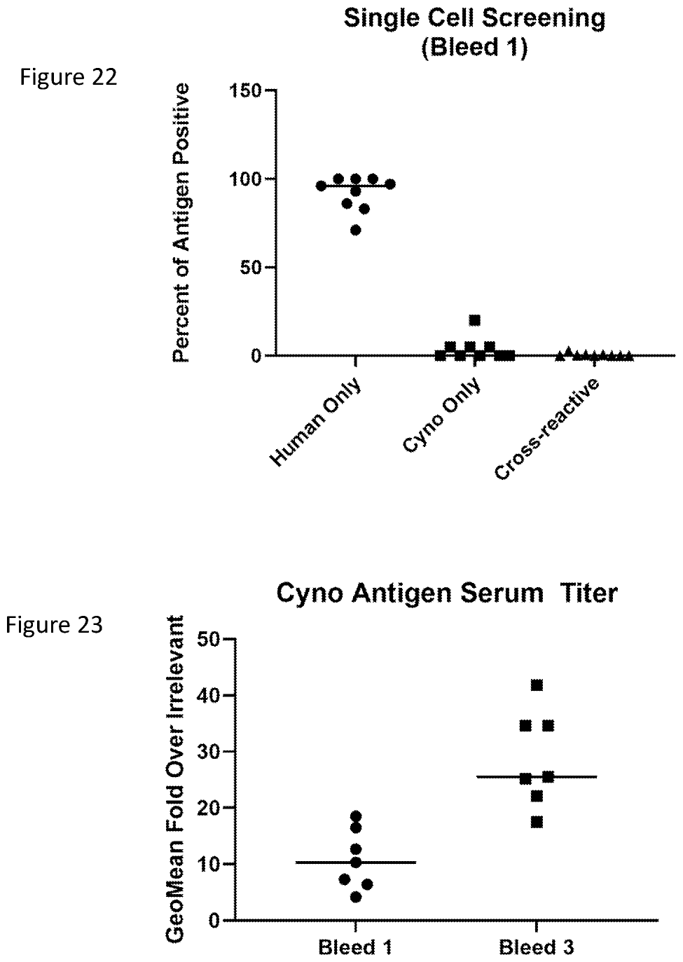

The serum titers from Bleed 1 were able to detect a robust immune response to the human antigen with low but detectable binding to the cyno antigen (FIG. 20). However, the polyclonal titer data could not differentiate between antibodies that only bound to the cyno antigen and those that could cross-react both human and cyno. By contrast, the single cell screen was able to detect binding to human only, cyno only and both human and cyno cross-reactive antibodies (FIGS. 21-22). Both serum titers and the single cell screen demonstrated that the dominant immune response was restricted to antibodies that only bound the human ortholog. While some cross-reactive antibodies were generated, most of the cyno response could not bind to the human ortholog and additional immunization was needed.

The mice were then boosted with a sub-domain of the human antigen (Antigen 2) that has a higher degree of human/cyno homology relative to the full-length protein. The mice initially received an injection of 25 sg of Antigen 2 with no adjuvant in the peritoneal cavity. The mice were then bled (Bleed 2), and the samples screened for binding to cyno and human orthologs. This boost did not produce a strong enough immune response to identify cross-reactive antibodies with confidence. Therefore, the mice were boosted one additional time with 25 sg of Antigen 2 combined with 50% SAS. The mice were bled four days after the boost (Bleed 3) and the CD138+ ASCs were isolated and screened as described above. This experiment highlights the value of using non-terminal sampling to allow for adjustments and boost extensions when needed.

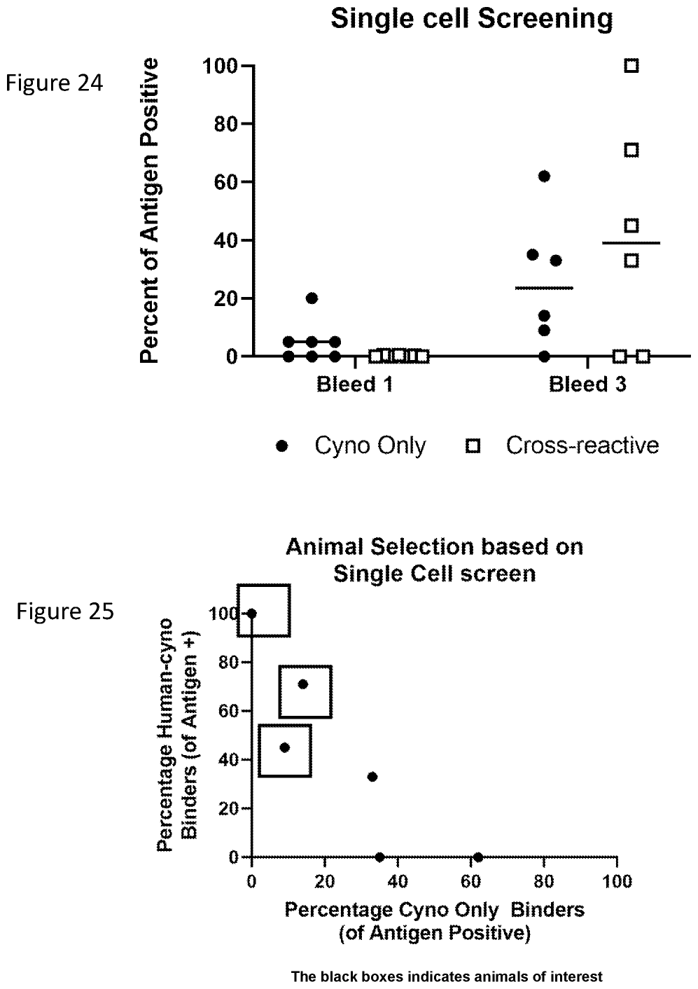

Boosting with Antigen 2 generated a robust increase in serum titers to the cyno ortholog of the antigen (FIG. 23). Consistent with this observation, the single cell assay also detected an increase in both cyno only and cyno-human cross-reactive antibodies (FIG. 24) in Bleed 3 relative to Bleed 1. Animals of interest could then be identified by plotting the percentage of cyno only binding versus binding to both human and cyno orthologs. This plot revealed that some mice generated a response to the cyno ortholog alone while other had robust cross-reactivity. This would not be discernable using standard polyclonal serology.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of monitoring for the production of select antibodies in a non-human animal, said method comprising
a. immunizing a non-human animal with an immunogen;
b. obtaining a blood sample comprising antibody secreting cells (ASCs) from said non-human animal; and
c. individually assaying ASCs present in the blood sample, or a fraction thereof, for the production of select antibodies.

2. A method of guiding antibody production in a non-human animal for the production of select antibodies, said method comprising:
a. performing an initial immunization on a non-human animal with an immunogen;
b. obtaining a blood sample comprising antibody secreting cells (ASCs) from said non-human animal;
c. individually assaying ASCs present in the blood sample, or a fraction thereof, for the production of select antibodies; and
d. performing a cycle of steps when the percentage of ASCs producing select antibodies is below a threshold, wherein the cycle comprises:
i. performing a subsequent immunization on the non-human animal with an immunogen when the percentage of ASCs producing select antibodies is below a threshold,
ii. obtaining a blood sample comprising ASCs from said non-human animal,
iii. individually assaying ASCs present in the blood sample, or a fraction thereof, for the production of select antibodies.

3. The method of claim 1 or 2, wherein the assaying comprises a single-cell, live-cell assay.

4. The method of claim 3, wherein multiple ASCs are simultaneously assayed.

5. The method of any one of the preceding claims, comprising applying the blood sample, or a fraction thereof, to a matrix and assigning a unique address of the matrix to each ASC.

6. The method of claim 5, wherein a result of the assaying is the identification of each ASC producing select antibodies.

7. The method of claim 6, wherein the result comprises the identification of the unique address of each ASC producing select antibodies.

8. The method of any one of claims 2-7, wherein the cycle is carried out at least one time.

9. The method of claim 8, wherein the cycle is repeated until the number of ASCs producing select antibodies, as assayed in (iii), is at or above the threshold.

10. The method of claim 9, wherein the cycle is repeated at least two times.

11. The method of any one of claims 2-10, wherein the immunogen of the subsequent immunization is different from the immunogen of the initial immunization.

12. The method of any one of claims 2-11, wherein each subsequent immunization differs from a prior immunization in that (A) a different immunogen, adjuvant, and/or immunomodulatory agent is administered to the non-human animal, (B) a different dose of the immunogen is administered to the non-human animal, (C) the time between each administration of the immunogen, adjuvant, immunomodulatory agent is different, and/or (D) the route of administration for each administration of immunogen, adjuvant, immunomodulatory agent is different.

13. The method of any one of claims 2-12, wherein a different immunogen is used each time the non-human animal is immunized.

14. A method of producing select antibodies in a non-human animal, comprising
a. performing an initial immunization on a non-human animal with an immunogen;
b. obtaining a blood sample comprising antibody secreting cells (ASCs) from said non-human animal;
c. individually assaying ASCs present in the blood sample, or a fraction thereof, for the production of select antibodies;
d. performing a cycle of steps when the percentage of ASCs producing select antibodies is below a threshold, wherein the cycle comprises:
i. performing a subsequent immunization on the non-human animal with an immunogen when the percentage of ASCs producing select antibodies is below a threshold,
ii. obtaining a blood sample comprising ASCs from said non-human animal,
iii. individually assaying ASCs present in the blood sample, or a fraction thereof, for the production of select antibodies, and
e. isolating the select antibodies and/or an ASC producing the select antibodies.

15. The method of claim 14, comprising determining the nucleotide sequence encoding the heavy chain variable region of the select antibodies produced by an ASC and the nucleotide sequence encoding the light chain variable region of the select antibodies produced by the ASC, introducing into a host cell a first vector comprising the nucleotide sequence encoding the heavy chain variable region of the select antibodies and a second vector comprising the nucleotide sequence encoding the light chain variable region of the select antibodies, and isolating the antibodies produced by the host cell.

16. The method of any one of the preceding claims, wherein the assaying comprises:
a. combining the ASCs within the matrix with (i) a capture reagent which binds to the select antibodies and comprises a solid support, (ii) a detection reagent which binds to the select antibodies and comprises a first detectable label, and (iii) a labeled target to which the select antibodies bind, wherein the labeled target comprises a second detectable label distinct from the first detectable label;
b. assaying for the first detectable label and for the second detectable label; and;
c. identifying the positions within the matrix at which both the first detectable label and the second detectable label are detected, wherein each identified position locates an individual ASC producing select antibodies.

17. The method of claim 16, wherein the capture agent comprises an antibody that binds to an antibody Fc domain attached to a solid support.

18. The method of claim 16 or 17, wherein the detection agent comprises an antibody that binds to an antibody Fc domain attached to a first detectable label.

19. The method of claim 18, wherein the antibody that binds to an antibody Fc domain of the capture agent is the same antibody of the detection agent.

20. The method of any one of claims 16-19, wherein the combining takes place in a well and the capture agent forms a monolayer in the well, optionally, wherein the ASCs are first exposed to the capture reagent, detection reagent, and/or labeled target in the well or immediately prior to being added to the well.

21. The method of claim 20, wherein the method comprises identifying the positions within the well at which both the first detectable label and the second detectable label are detected, wherein each identified position locates an individual ASC producing select antibodies.

22. The method of any one of claims 16-19, wherein the combining takes place in a microfluidic or nanofluidic chamber, a microwell or nanowell device, a microcapillary or nanocapillary tube, or a nanopen of a nanofluidic chip.

23. The method of claim 22, wherein the combining takes place in a nanopen of a nanofluidic chip.

24. The method of claim 23, wherein the method comprises identifying the position of each pen within the nanofluidic chip at which both the first detectable label and the second detectable label are detected, wherein each identified position locates an individual ASC producing select antibodies.

25. The method of claim 23 or 24, wherein a single ASC of the blood sample is moved into a pen of the nanofluidic chip through optoelectro positioning (OEP).

26. The method of any one of the preceding claims, wherein the select antibodies bind to a target which is the same as or similar to the immunogen used to immunize the non-human animal.

27. The method of claim 26, wherein the select antibodies bind to the target in the presence of one or more competitive binding agents.

28. The method of claim 27, wherein the competitive binding agents are combined with the ASCs, capture reagent, detection reagent, and labeled target during the assaying.

29. The method of any one of the preceding claims, wherein the select antibodies bind to a target with a target affinity, optionally, wherein the KD of the select antibodies for the target is about 10⁻¹¹ M to about 10⁻⁹ M.

30. The method of claim 29, wherein the assaying is carried out in a first round with a first amount of the labeled target and a second round with a second amount of the labeled target, wherein the first amount is greater than the second amount, optionally, wherein the assaying is further carried out in a third round with a third amount of the labeled target and the third amount is less than the second amount, wherein when the ASC binds to the labeled target in each round, the ASC produces select antibodies.

31. The method of any one of the preceding claims, wherein the select antibodies bind to a target and to an ortholog or paralog thereof, optionally, wherein the target is a human protein and the ortholog is a cynomolgus monkey protein.

32. The method of claim 31, wherein a second labeled target is combined with the ASCs, capture reagent, detection reagent, and labeled target, wherein the second labeled target comprises the ortholog attached to a third detectable label which is distinct from the first detectable label and the second detectable label, wherein the method further comprises assaying for the third detectable label and identifying the position(s) at which the first detectable label, the second detectable label, and the third detectable label are detected, wherein each identified position locates an individual ASC producing select antibodies.

33. The method of any one of the preceding claims, wherein the select antibodies bind to a target and not to an ortholog or paralog thereof.

34. The method of claim 33, wherein a second labeled target is combined with the ASCs, capture reagent, detection reagent, and labeled target, wherein the second labeled target comprises the ortholog attached to a third detectable label which is distinct from the first detectable label and the second detectable label, wherein the method further comprises assaying for the third detectable label and identifying the position(s) at which only the first detectable label and the second detectable label, but not the third detectable label, are detected, wherein each identified position locates an individual ASC producing select antibodies.

35. The method of any one of the preceding claims, wherein the select antibodies bind to a portion of the target.

36. The method of claim 35, wherein a second labeled target is combined with the ASCs, capture reagent, detection reagent, and labeled target, wherein the second labeled target comprises the portion of the target attached to a third detectable label which is distinct from the first detectable label and the second detectable label, and wherein the method further comprises assaying for the third detectable label and identifying the position(s) at which the first detectable label, the second detectable label, and the third detectable label are detected, wherein each identified position locates an individual ASC producing select antibodies.

37. The method of claim 36, wherein the target is a protein comprising multiple domains and the select antibodies bind to only one domain of the target, wherein the labeled target comprises the extracellular domain of the target attached to the second detectable label and the second labeled target comprises the one domain attached to third detectable label.

38. The method of any one of the preceding claims, wherein the select antibodies bind to a conformational epitope formed upon dimerization or multimerization of the target and the target comprises a dimerization domain or multimerization domain.

39. The method of claim 38, wherein the labeled target comprises the extracellular domain of the immunogen attached to the second detectable label, wherein a second labeled target is combined with the ASCs, capture reagent, detection reagent, and labeled target, wherein the second labeled target comprises the dimerization domain or multimerization domain of the immunogen attached to the third detectable label which is distinct from the first detectable label and the second detectable label, and wherein the method further comprises assaying for the third detectable label and identifying the position(s) at which the first detectable label, the second detectable label, and the third detectable label are detected, wherein each identified position locates an individual ASC producing select antibodies.

40. The method of any one of the preceding claims, wherein the blood sample is obtained from the non-human animal about 3 to about 7 days after the immunizing step.

41. The method of any one of the preceding claims, wherein the blood sample obtained from the non-human animal is less than or about 500 μL.

42. The method of claim 41, wherein the blood sample is about 100 μL to about 250 μL.

43. The method of any one of the preceding claims, wherein the ASCs are CD138+ B cells.

44. The method of any one of the preceding claims, wherein the ASCs comprise migratory plasmablasts.

45. The method of any one of the preceding claims, further comprising removing one or more components of the blood sample obtained from the non-human animal prior to assaying.

46. The method of claim 45, wherein red blood cells, plasma, and/or platelets are removed from the blood sample.

47. The method of claim 45 or 46, wherein the fraction of the blood sample is prepared by selecting for CD138⁺ cells.

48. The method of any one of the preceding claims, wherein the non-human animal is subjected to neither removal of one or more secondary lymphoid organs nor euthanasia.

49. The method of any one of the preceding claims, wherein ASCs from the blood sample are not used in making hybridomas.

50. The method of any one of claims 2 to 49, wherein the non-human animal is one of a series of non-human animals, and an outcome of the assaying is the identification of the non-human animals having a number of ASCs producing select antibodies below the threshold and/or requiring further immunization.

51. The method of any one of claims 2 to 50, wherein the steps of the method are carried out on a series of non-human animals and the method comprises profiling the B-cell repertoire of the blood sample for each non-human animal of the series and selecting a subset of the series having a target B-cell profile.

52. The method of any one of the preceding claims, comprising sacrificing the non-human animal and harvesting tissues from the non-human animal, when the percentage of ASCs producing select antibodies is at or above a threshold.

53. The method of claim 52, comprising harvesting the spleen from the non-human animal.

54. The method of claim 53, comprising screening for B-cells of the spleen and/or generating hybridomas from cells of the spleen.

55. A method of screening a series of non-human animals for antibody secreting cells (ASCs) producing select antibodies, said method comprising:
monitoring for the production of select antibodies in a non-human animal in a series of non-human animals in accordance with the method of any one of the preceding claims,
wherein for each non-human animal of the series the number of ASCs producing the select antibodies is identified.

56. The method of claim 55, wherein when the percentage of ASCs producing select antibodies for an animal is below a threshold, the method comprises performing a subsequent immunization.

57. The method of claim 56, wherein when the percentage of ASCs producing select antibodies for an animal is at or above a threshold, the method further comprises harvesting secondary lymphoid organs from the animal.

58. A method of selecting immunized non-human animals for subsequent immunization, said method comprising:
monitoring for the production of select antibodies in a non-human animal in accordance with the method of any one of the preceding claims,
wherein for each non-human animal, the number of ASCs producing the select antibodies is identified, and
selecting the animal for subsequent immunization when the percentage of ASCs producing select antibodies for an animal is below a threshold.

59. A method of selecting immunized non-human animals for euthanasia and secondary lymphoid harvest, said method comprising:
monitoring for the production of select antibodies in a non-human animal in accordance with the method of any one of the preceding claims,
wherein for each non-human animal, the number of ASCs producing the select antibodies is identified, and
selecting the animal for euthanasia and secondary lymphoid harvest, when the percentage of ASCs producing select antibodies for an animal is at or above a threshold.

60. A method of assaying for ASCs producing select antibodies, said method comprising:
a. combining in a well (i) a blood sample obtained from a non-human animal immunized with an immunogen, or a fraction thereof, wherein the blood sample comprises antibody secreting cells (ASCs), (ii) a detection reagent which binds to the select antibodies and comprises a first detectable label, and (iii) a target to which the select antibodies bind,
wherein:
(A) the target is a labeled target comprising a second detectable label distinct from the first detectable label and a capture reagent which binds to the select antibodies and comprises a solid support is further combined in the well to form a monolayer in the well,
or
(B) the target is expressed on the surface of cells and the cells are combined in the well to form a monolayer in the well,
b. assaying for the first detectable label and optionally assaying for the second detectable label, when the target is a labeled target;
c. identifying the positions within the well at which the first detectable label is detected or the first and second detectable labels are detected, wherein each identified position locates an individual ASC producing select antibodies.

61. The method of claim 60, wherein the ASCs are first exposed to the detection reagent and/or target in the well or immediately prior to being added to the well.

62. The method of claim 60 or 61 wherein the select antibodies bind to a target which is the same as or similar to the immunogen used to immunize the non-human animal.

63. The method of any one of claims 60-62, wherein the detection reagent comprises an antibody that binds to an antibody Fc domain attached to a solid support and/or the detection agent comprises an antibody that binds to an antibody Fc domain attached to a first detectable label

64. The method of claim 63, wherein the antibody that binds to an antibody Fc domain of the capture agent is the same antibody of the detection agent.

65. The method of any one of claims 60-64, wherein the blood sample is obtained from the non-human animal about 3 to about 7 days after the immunizing step.

66. The method of any one of the preceding claims, wherein the blood sample obtained from the non-human animal is less than or about 500 μL, optionally, about 100 μL to about 250 μL

67. The method of any one of claims 60-66, wherein the ASCs are CD138+ B cells.

68. The method of any one of claims 60-67, wherein the ASCs comprise migratory plasmablasts.

69. The method of any one of claims 60-68, further comprising removing one or more components of the blood sample obtained from the non-human animal prior to combining in the well.

70. The method of claim 69, wherein red blood cells, plasma, and/or platelets are removed from the blood sample.

71. The method of claim 69 or 70, wherein the fraction of the blood sample is prepared by selecting for CD138⁺ cells.

72. The method of any one of claims 60-71, wherein the select antibodies bind to the target in the presence of one or more competitive binding agents.

73. The method of claim 72, wherein the competitive binding agents are combined with the ASCs, detection reagent, and cells expressing the target during the assaying.

74. The method of any one of claims 60-73, wherein the select antibodies bind to a target with a target affinity, optionally, wherein the KD of the select antibodies for the target is about 10⁻¹¹ M to about 10⁻⁹ M.

75. The method of claim 74, wherein the assaying is carried out in a first round with a first amount of cells expressing the target and a second round with a second amount of the cells expressing the target, wherein the first amount is greater than the second amount, optionally, wherein the assaying is further carried out in a third round with a third amount of the cells expressing the target and the third amount is less than the second amount, wherein when the ASC binds to the labeled target in each round, the ASC produces select antibodies.

76. The method of any one of claims 60-75, wherein the select antibodies bind to a target and to an ortholog or paralog thereof, optionally, wherein the target is a human protein and the ortholog is a cynomolgus monkey protein.

77. The method of claim 76, wherein the cells express the target and the ortholog or paralog thereof.

78. The method of any one of claims 60-77, wherein the select antibodies bind to a target and not to an ortholog or paralog thereof.

79. The method of any one of claims 60-78, wherein the select antibodies bind to a portion of the target.

80. The method of claim 79, wherein the target is a protein comprising multiple domains and the select antibodies bind to only one domain of the target, wherein the labeled target comprises the extracellular domain of the target attached to the second detectable label and the second labeled target comprises the one domain attached to third detectable label.

81. The method of any one of claims 60-80, wherein the select antibodies bind to a conformational epitope formed upon dimerization or multimerization of the target and the target comprises a dimerization domain or multimerization domain.
